(19) United States
(12) Patent Application Publication
(10) Pub. No.: US 2025/0332290 A1
(43) Pub. Date: Oct. 30, 2025

(54) PSMA-TARGETING LIGANDS FOR MULTIMODAL APPLICATIONS

(71) Applicants: STICHTING RADBOUD UNIVERSITAIR MEDISCH CENTRUM, Nijmegen (NL); STICHTING RADBOUD UNIVERSITEIT, Nijmegen (NL)

(72) Inventors: Mark RIJPKEMA, Nijmegen (NL); Sandra HESKAMP, Nijmegen (NL); Peter LAVERMAN, Nijmegen (NL); Yvonne Hendrika Wilhelmina DERKS, Nijmegen (NL); Dennis Wilhelmus Petrus Maria LOWIK, Nijmegen (NL)

(73) Assignees: STICHTING RADBOUD UNIVERSITAIR MEDISCH CENTRUM, Nijmegen (NL); Stichting Radboud Universiteit, Nijmegen (NL)

(21) Appl. No.: 18/263,141

(22) PCT Filed: Feb. 8, 2022
(86) PCT No.: PCT/EP2022/052988

§ 371 Date: Jul. 27, 2023

(30) Foreign Application Priority Data
Feb. 8, 2021 (EP) 21155853.1

(51) Int. Cl. A61K 49/00; A61K 41/00; A61P 35/00

(52) U.S. Cl. CPC A61K 49/0056; A61K 41/0057; A61K 49/0032; A61P 35/00; A61K 2121/00; A61K 2123/00

(57) ABSTRACT

The invention relates to a new class of compounds that can serve as prostate specific membrane antigen (PSMA) ligands, and to precursors to this class. The precursors have an amine that is available for functionalisation. Derivatives of the compounds are useful for imaging and therapy of cancer.

### FIELD OF THE INVENTION

The invention relates to a new class of compounds that can serve as prostate specific membrane antigen (PSMA) ligands, and to precursors to this class. The precursors have an amine that is available for functionalisation. Derivatives of the compounds are useful for imaging and therapy of cancer.

### BACKGROUND ART

Despite recent improvements in imaging and therapy, prostate cancer (PCa) remains the most frequently diagnosed cancer type in men and is estimated to be the second leading cause of cancer-related deaths. One of the curative options in early stage prostate cancer treatment is surgical removal of all cancerous tissue: radical prostatectomy with or without pelvic lymph node dissection. In surgical treatment, incomplete resection of PCa and understaging of possible undetected metastases may lead to disease recurrence and consequently poor patient outcome. Prostate specific membrane antigen (PSMA) targeting agents may aid the surgeon in intraoperative treatment of PCa lesions.

To improve the surgical treatment of cancer the inventors developed a precursor that can be converted into a multimodal PSMA-targeting ligand that comprises a PSMA-binding motif, a fluorophore/photosensitizer such as IRDye700DX, and a chelator for radiolabeling such as DOTA. The fluorophore/photosensitizer can be used for tumor-targeted photodynamic therapy (tPDT). tPDT is a highly selective cancer treatment based on targeting molecules conjugated to photosensitizers that, after exposure to near infrared light, transform oxygen into radical oxygen species (ROS) toxic for the tumor cell. During surgery, tPDT can be used for treatment of any remaining unresectable lesions and for the irradiation of micrometastases in the wound bed. Moreover, intraoperative fluorescence imaging of the IRDye700DX can be used to visualize and delineate the primary tumor. The simultaneously present chelator renders the compound suitable for preoperative SPECT/CT imaging and radiodetection of metastatic lymph nodes. By using a multimodal ligand, only one tracer injection is needed for the entire (surgical) treatment of PCa.

Surgical treatment of cancer such as PCa faces two main challenges. First, complete resection of the primary prostate tumor (achieving negative surgical margins) and its metastases remains difficult. Removal of the entire prostate gland often leads to nerve damage that may cause debilitating functional side effects such as urinary incontinence and erectile dysfunction. Therefore, surgeons often have to choose between attempting complete oncological resection and nerve-saving operations with a maximum chance of good functional outcome, but a higher chance of positive resection margins as a consequence. For instance, tumor lesions (or parts of it) can be difficult to resect due to proximity to other tissues, such as the nerves, blood vessels, bladder and rectum. In addition, lesions or positive surgical margins might be too small in size to be picked up with the intraoperative imaging techniques. This would mean that very small amount of tumor are missed by the surgeon and remain in situ, leading to disease recurrence after a few years. The average rate of positive surgical margins after radical prostatectomy is 15% and can increase up to 50% in men with more locally advanced disease (Lutje et al., J Nucl Med. 2014; 55 (6): 995-1001). The effect of incomplete resection can be profound for the individual patient as it may lead to early disease recurrence and poor patient outcome. Hence, margin detection and intraoperative treatment of any remaining positive tumor margins using tPDT is of utmost importance for the surgical management and clinical outcome of PCa patients.

The second challenge in the surgical treatment of PCa is the detection of tumor-positive lymph nodes (Nagaya et al., Front Oncol. 2017; 7:314). The ability to find and resect metastatic lymph node tissue is currently based on anatomical landmarks like the obturator region together with the external and internal ileac artery region. Intraoperative tissue shifts, atypical lymph node locations, small lymph node size and inconspicuous morphology hamper the detection of tumor positive lymph nodes during surgery and thus their resection.

To improve surgical treatment of PCa, recent developments have focused on prostate-specific membrane antigen (PSMA) targeting ligands (Pinto et al., Clin Cancer Res. 1996; 2 (9): 1445-51; Pomper et al., Mol Imaging. 2002; 1 (2): 96-101). PSMA represents an excellent target for imaging of PCa, as its expression is drastically elevated in 90-100% of local PCa lesions, tumorous lymph nodes, and metastatic bone lesions. PSMA ligands usually comprise a PSMA-binding motif (glutamate-urea-lysine, KuE) which, via the lysine side chain, can be functionalized with different imaging/therapeutic agents; for example, near-infrared fluorescent (NIRF) dyes for optical/fluorescence imaging, photosensitizers for targeted photodynamic therapy of prostate cancer or chelators for radionuclide labeling.

Achieving complete resection of tumor tissue is challenging. For example, when lymph nodes or positive tumor margins are located in close proximity to surrounding healthy tissue (e.g. nerves, blood vessels, bladder and rectum). These difficult to resect tumor lesions can potentially be eradicated by targeted photodynamic therapy (tPDT). Three components needed for tPDT are a light, oxygen and a photosensitizer. Upon activation, the photosensitizer undergoes an oxygen-mediated photochemical process producing reactive oxygen species (ROS) which results in specific cellular damage of the target cells (Liu et al., Prostate. 2009; 69 (6): 585-94). In addition, tPDT may even lead to systemic immunity due to destruction of tumor cells inducing an anti-tumor immune response. As PSMA-targeted tracers with a photosensitizer are designed to accumulate in PCa lesions and the light (normal or laparoscopic 680 nm laser) can be focused to the tumor site as well, tPDT is highly specific. Potentially, it enables therapy with minimal side effects.

Many PSMA ligands are known, for instance from WO2015055318 and WO2017054907. Besides the urea motif, it is known that negative charges improve the PSMA-binding and tumor-to-background ratios of related PSMA ligands (K. Bao et al., Chem Commun (Camb) 53, 1611-1614 (2017); S. S. Huang et al., Prostate 74, 702-713 (2014)).

PSMA ligands that are used in the clinic and have been described before were, until now, only functionalized with chelators for radionuclide labelling. These ligands can thus only be used for diagnostic PET/CT scanning, preoperative SPECT/CT scanning, intraoperative localization of tumor lesions with a gamma probe or portable SPECT camera (radioguided surgery) and radioligand therapy (e.g. ¹⁷⁷Lu-PSMA), but not for fluorescence imaging or PDT.

PSMA ligands with a photosensitizer attached to it have been developed before, including the tracers called PSMA-1-IR700 (Wang et al., Mol Cancer Ther. 2016; 15 (8): 1834-44) and YC9 (Chen et al., J Photochem Photobiol B. 2017; 167:111-6). Nonetheless, these tracers were not multimodal and lacked a chelator. Lutje et al. (Theranostics. 2019; 9 (10): 2924-38) developed a multimodal anti-PSMA antibody conjugated with both a chelator and a dye. However, an antibody shows completely different kinetics and requires that it for instance be humanized before clinical translation is feasible, which is not the case for multimodal small molecule PSMA-ligands.

There is a need for improved PSMA ligands. There is a need for PSMA ligands with good or improved binding characteristics that can nonetheless be further derivatized. There is a need for improved multimodal PSMA probes. There is a need for improved PSMA ligands that can be used for imaging. There is a need for improved PSMA ligands that can be used for treatment.

### SUMMARY OF THE INVENTION

To solve this problem, the inventors developed precursors for multimodal probes, featuring a positively charged amine in a region of the PSMA-ligand that is conventionally negatively charged. This amine allows further derivatization with chelators, labels, or bifunctional linkers to allow the derivatization with multiple different types of groups. FIG. 1 demonstrates the difference between an embodiment of the invention and known PSMA ligands, in this case ¹⁸F-PSMA-1007. Thus the invention provides a compound of general formula (1) or a salt thereof:

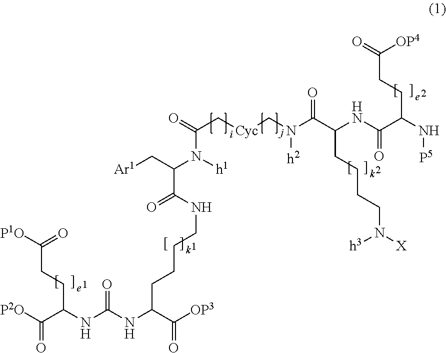

wherein P¹, P², P³, P⁴, and P⁵ are each independently H or a protecting group; e¹ and e² are each independently 1 or 2; k¹ and k² are each independently 0, 1 or 2; i is 0 or 1; j is 0 or 1; h¹, h², and h³ are each independently H or CH₃; Ar¹ is an aromatic or heteroaromatic C5-12 hydrocarbon; Cyc is an aromatic, heteroaromatic, cyclic, or heterocyclic C5-10 hydrocarbon; X is H, a protecting group, a chelator, a detectable label, a pharmaceutically active agent, an albumin-binding moiety, or a linker, wherein the linker is optionally attached to a chelator, a detectable label, a pharmaceutically active agent, or two or more of a chelator, a detectable label, and a pharmaceutically active agent. Preferably P¹, P², P³, and P⁴, are each independently H or a protecting group that is a C1-7 hydrocarbon; or P⁵ is a C2-8 acyl group, preferably comprising a C5-6 aromatic or heteroaromatic ring; or e¹ is 1; or e² is 1; or k¹ is 1; or k² is 1; or i is 0; or j is 1; or h¹ is H; or h² is H; or h³ is H; or Ar¹ is naphthyl, phenyl, biphenyl, indolyl, benzothiazolyl, or quinoyl; or Cyc is a C5-10 aryl, a C6-10 alkylaryl, cyclopentyl, cyclohexyl, cycloheptyl, or piperidyl. In preferred embodiments of the compound P¹, P², P³, and P⁴, are each H or tert-butyl; or P⁵ is benzoyl, picolinyl, nicotinyl, or isonicotinyl; or e¹ and e² are 1; or k¹ and k² are 1; or i is 0 and j is 1; or h¹, h² and h³ are H; or Ar¹ is naphthyl; or Cyc is phenyl, cyclopentyl, cyclohexyl, cycloheptyl, or piperidyl. In preferred embodiments the compound is of general formula (1-L):

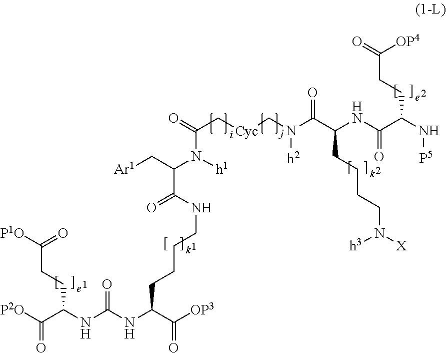

In preferred embodiments X is a chelator, a detectable label, or a linker, wherein the linker is optionally attached to a chelator, a detectable label, or both a chelator and a detectable label. Preferably the chelator is 1,4,7,10-tetraazacyclododecane-tetraacetic acid (DOTA) 1,4,7-triazacyclononane-triacetic acid (NOTA), triazacyclononane-phosphinate (TRAP), 1,4,8,11-tetraazacyclotetradecane-1,4,8,11-tetraacetic acid (TETA), N,N′-bis[2-hydroxy-5-(carboxyethyl)benzyl] ethylenediamine-N,N′-diacetic acid (HBED-CC), or diethylenetriaminepentaacetic anhydride (DTPA); and/or the detectable label is a fluorophore, a chromophore, a radiolabel, a specific isotope, a diagnostic marker, or a hapten, wherein a fluorophore is preferably fluorescein or its derivatives such as FITC or Tokyo green, ASP (preferably 4-(4-(didecylamino) styryl)-N-methylpyridinium iodide), rhodamine, Cyanine5 (also known as Cy5), Cyanine5.5 (also known as Cy5.5), Cyanine7 (also known as Cy7), sulfoCyanine7 (also known as sulfoCy7), Cyanine7.5 (also known as Cy7.5), IRDye700DX, IRDye 800CW, IRDye 800ZW, Alexa660, Alexa680, Alexa700, Alexa750, Alexa790, DyLight 755, DyLight 800 Fluoprobes 752, FluoProbes 782, calcein, any other Alexa-label, any other Cyanine-label, and sulfonated or otherwise modified variants of any of these fluorophores. The linker preferably comprises an amino acid, an oligo (ethylene glycol), or a C2-12 hydrocarbon, wherein the linker comprises at least one functional group for further modification, wherein the linker preferably comprises two functional groups for further modification. Preferably the compound comprises both a chelator and a detectable label.

Preferably the compounds are for use as a medicament. The medicament is preferably for treating a cancer and/or a metastasis thereof, preferably wherein the cancer is a prostate cancer or a salivary gland cancer, more preferably a prostate cancer. The medicament can be for imaging, diagnosing, and/or treating a cancer and/or a metastasis thereof. Also provided is a composition comprising such a compound, and its use as a medicament. Also provided is a method of imaging, diagnosing, or treating cancer in a subject in need thereof, the method comprising the step of administering a compound or a composition as described above to the subject.

### DESCRIPTION OF EMBODIMENTS

### Compounds

The invention provides a compound of general formula (1) or a salt thereof:

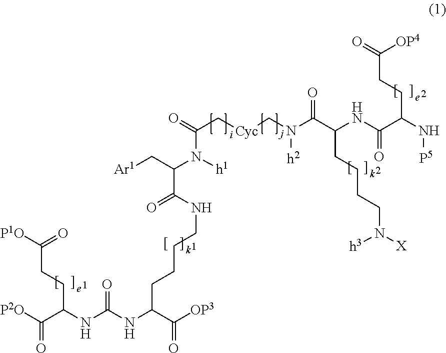

wherein
P¹, P², P³, P⁴, and P⁵ are each independently H or a protecting group;
e¹ and e² are each independently 1 or 2;
k¹ and k² are each independently 0, 1 or 2;
i is 0 or 1;
j is 0 or 1;
h¹, h², and h³ are each independently H or CH₃;
Ar¹ is an aromatic or heteroaromatic C5-12 hydrocarbon;
Cyc is an aromatic, heteroaromatic, cyclic, or heterocyclic C5-10 hydrocarbon;
X is H, a protecting group, a chelator, a detectable label, a pharmaceutically active agent, an albumin-binding moiety, or a linker, wherein the linker is optionally attached to a chelator, a detectable label, a pharmaceutically active agent, or two or more of a chelator, a detectable label, and a pharmaceutically active agent. Such a compound is referred to hereinafter as a compound according to the invention.

A compound according to the invention can broadly be divided in three regions. There is a urea-based region which comprises p¹, p², p³, e¹, and k¹. There is a hydrophobic bridging region which comprises Ar¹, h¹, h², i, j, and cyc. There is a charged region which comprises k², e², P⁴, P⁵, h³, and X.

A salt of a compound of general formula (1) is preferably a TFA salt, an HCl salt, a sodium salt, or another pharmaceutically acceptable salt. A salt is preferably a base addition salt wherein at least one of P¹, P², P³, and P⁴ is absent and a cationic counterion is present. In other words, as a skilled person will understand, P¹, P², P³, and P⁴ could be said to represent such a counterion, preferably cationic, wherein for instance the O to which P¹, P², P³, and P⁴ are attached is negatively charged. Examples of suitable salts are non-metallic salts such as ammonia salts, and metallic salts such as sodium salts and potassium salts. A skilled person can select suitable salt forms, and their means of production are well known (see e.g. “Occurrence of pharmaceutically acceptable anions and cations in the Cambridge Structural Database” Haynes et al., DOI: 10.1002/jps.20441). A salt can also be an acid addition salt, for instance when X or P⁵ are H. Acid addition salts are known in the art and examples are HCl salts, TFA salts, formic acid salts, and acetic acid salts.

P¹, P², P³, P⁴, and P⁵ are each independently H or a protecting group. In some embodiment P¹ and P² are identical. In some embodiments P¹, P², and P³ are identical. In preferred embodiments, P¹, P², P³, and P⁴ are identical. P⁵ is linked to a nitrogen atom where P¹, P², P³, and P⁴ are linked to an oxygen atom. Therefore, when P⁵ is a protecting group, it is unlikely to be identical to the other protecting groups, if any.

P¹, P², P³, and P⁴ can contribute to forming carboxylic acids when they are H, or can represent protecting groups for carboxylic acids. Such groups can later be converted into carboxylic acids, and are known in the art. Preferably, P¹, P², P³, and P⁴ are in each instance independently chosen from hydrogen and a C1-7 hydrocarbon protecting group, which can be a linear, branched, or cyclic C1-7acyl or alkyl wherein each carbon atom is optionally substituted by a halogen, an alkoxy (preferably C1-3alkoxy), or a haloalkoxy (preferably C1-3haloalkoxy) moiety, and wherein the acyl or alkyl is optionally unsaturated. Preferred protecting groups for carboxylic acids are formed when the C1-7 hydrocarbon is methyl, tert-butyl, or benzyl, of which tert-butyl is most preferred. For compounds with more direct activity, it is preferred that P¹, P², P³, and P⁴ are H. For compounds for which further synthetic procedures are desired prior to any clinical use, it is preferred that P¹, P², P³, and P⁴ are a protecting group, more preferably a linear, branched, or cyclic C1-7acyl or alkyl as described above. In preferred embodiments, P¹, P², P³, and P⁴ are in each instance independently chosen from hydrogen and a linear C1-4acyl wherein each carbon atom is optionally substituted by a halogen or a methoxy moiety. Most preferably P¹, P², P³, and P⁴ are H or tert-butyl.

P⁵ can contribute to forming an amine when it is H, or it can represent a protecting group for such an amine. Such groups can later be converted into free amines, and are known in the art. Preferably, P⁵ is chosen from hydrogen, a C1-14 hydrocarbon protecting group, a —C(═O)—O—(C1-14 hydrocarbon) protecting group, or an —S(═O)₂—(C1-14 hydrocarbon) protecting group, wherein a C1-14 hydrocarbon can be a linear, branched, or cyclic C1-14acyl or alkyl, preferably alkyl, wherein each carbon atom is optionally substituted by a halogen, an alkoxy (preferably C1-3alkoxy), or a haloalkoxy (preferably C1-3haloalkoxy) moiety, and wherein the acyl or alkyl is optionally unsaturated. Preferred protecting groups for amines are formed when P⁵ is benzyl, benzyl carbamate, benzoyl, nicotinyl, tert-butyl carbamate, 9-fuorenylmethyl carbamate, tosyl, —C(phenyl)₃, trifluoroacetyl, acetyl, or a phthalimide forming moiety, of which benzyl, benzyl carbamate, benzoyl, nicotinyl, are more preferred, and benzoyl and nicotinyl are most preferred. Also preferred are benzoyl, picolinyl, nicotinyl, and isonicotinyl. Herein, a preferred nicotinyl is preferably 3-nicotinyl. For compounds with more direct activity, it is preferred that P⁵ is H or even more preferably benzoyl or nicotinyl. For compounds for which further synthetic procedures are desired prior to any clinical use, it is preferred that P⁵ is a protecting group, more preferably benzoyl and nicotinyl. Highly preferably P⁵ is H or benzoyl or nicotinyl, most preferably benzoyl or nicotinyl. Instances of benzoyl and nicotinyl can be optionally substituted with halogen, C1-3alkyl, C1-3haloalkyl, or C1-3alkoxy.

A hydrocarbon as used herein has the amount of carbon atoms as indicated, and can be an alkyl or acyl moiety, either cyclic, linear, or branched, as described herein. It can be saturated or unsaturated, and it can be optionally substituted as described above. When it is unsaturated and cyclic, it is preferably aromatic. A hydrocarbon can comprise heteroatoms, preferably selected from N, O, and S. An aromatic hydrocarbon can therefore also be heteroaromatic. A cyclic hydrocarbon can therefore also be heterocyclic. A preferred hydrocarbon is a phenyl moiety.

If not stated otherwise, the term “alkyl” by itself or as part of another molecule, means a straight or branched chain, or cyclic hydrocarbon radical, or combination thereof, which may be fully saturated, mono- or polyunsaturated and can include di- and multivalent radicals, The “alkyl” residue is preferably C1-10 (unless otherwise indicated0 and may be unsubstituted or substituted (e.g with halogen). Preferred alkyl residues are methyl, ethyl, n-propyl, iso-propyl, n-butyl, tert-butyl, n-pentyl, n-hexyl, n-hepyl or n-octyl or the like. The same also applies to the corresponding cycloalkyl compounds having preferably 3 to 10 carbon atoms, e.g. cycloproyl, cyclobutyl, cyclopentyl, cyclohexyl, cycloheptyl, cyclooctyl, etc., An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl, such as “heteroalkyl”, “haloalkyl” and “homoalkyl”. A preferred heteroalkyl is an alkoxyl.

e¹ and e² are each independently 1 or 2. In some embodiments, both e¹ and e² are 1. In some embodiments, both e¹ and e² are 2. In some embodiments, e¹ is 1 and e² is 2. In some embodiments, e¹ is 2 and e² is 1. Preferably, at least e¹ is 1, and e² is 1 or 2. It is most preferred that both e¹ and e² are 1, which would contribute to the formation of a glutamate residue. e¹ is most preferably 1. e² is most preferably 1.

k¹ and k² are each independently 0, 1 or 2. In some embodiments, both k¹ and k² are 0. In some embodiments, both k¹ and k² are 1. In some embodiments, both k¹ and k² are 2. In preferred embodiments, k¹ is 1 and k² is 0, 1, or 2. Preferably, at least k¹ is 1, and k² is 1 or 2. It is most preferred that both k¹ and k² are 1, which would contribute to the formation of a lysine residue. k¹ is most preferably 1. k² is most preferably 1.

i is 0 or 1. Most preferably it is 0. i is connected to a cyclic moiety that is part of the hydrophobic region in between the urea-motif and the charged motif. j is 0 or 1. Most preferably it is 1. j is connected to the same cyclic moiety as i, when both are present. In some embodiments, both i and j are 1. In some embodiments, both i and j are 0. In some embodiments, i is 1 and j is 0. In some embodiments, i is 0 and j is 1. Preferably, at least i is 1, and j is 0 or 1. It is most preferred that i is 0 and j is 1.

h¹, h², and h³ are each independently H or CH₃. In preferred embodiments, each of h¹, h², and h³ represent the same moiety. In some embodiments, h¹, h², and h³ are each CH₃. In preferred embodiments, h¹, h², and h³ are each H. In some embodiments h¹ is H and h² and h³ are CH₃. In some embodiments h¹ and h² are H and h³ is CH₃. In some embodiments h¹ is CH3 and h² and h³ are H. In some embodiments h¹ and h² are CH₃ and h³ is H. In some embodiments h¹ and h³ are H and h² is CH₃. In some embodiments h¹ and h³ are CH₃ and h² is H.

Ar¹ is an aromatic or heteroaromatic C5-12 hydrocarbon. An aromatic hydrocarbon can also be referred to as “aryl”, and, as used herein, refers to a closed ring structure which has at least one ring having a conjugated pi electron system and includes both carbocyclic aryl and heterocyclic aryl (or “heteroaryl” or “heteroaromatic”) groups. The carbocyclic or heterocyclic aromatic group may contain from 5 to 12 ring atoms. The term includes monocyclic rings linked covalently or fused-ring polycyclic (i.e., rings which share adjacent pairs of carbon atoms) groups. An aromatic group can be unsubstituted or substituted. Non-limiting examples of “aromatic” or “aryl”, groups include phenyl, 1-naphthyl, 2-naphthyl, I, 2-biphenyl, 3-biphenyl, 4-biphenyl, anthracenyl, and phenanthracenyl. Substituents for each of the above noted aryl and heteroaryl ring systems are selected from the group of acceptable substituents (e.g. alkyl, carbonyl, carboxyl, alkoxyl, or halogen) described herein. The term “aryl” when used in combination with other terms (including but not limited to, aryloxy, arylthioxy, aralkyl) includes both aryl and heteroaryl rings. Thus, the term “aralkyl” or “alkaryl” is meant to include those radicals in which an aryl group is attached to an alkyl group (including but not limited to, benzyl, phenethyl, pyridylmethyl and the like) including those alkyl groups in which a carbon atom (including but not limited to, a methylene group) has been replaced by a heteroatom, by way of example only, by an oxygen atom. Examples of such aryl groups include, but are not limited to, phenoxymethyl, 2-pyridyloxymethyl, 3-(I-naphthyloxy) propyl, and the like.

Heteroaromatic hydrocarbon can also be reffered to as “heteroaryl”, and, as used herein, refers to aryl groups which contain at least one heteroatom selected from N, O, and S; wherein the nitrogen and sulfur atoms may be optionally oxidized, and the nitrogen atom(s) may be optionally quaternized. Heteroaryl groups may be substituted or unsubstituted. A heteroaryl group may be attached to the remainder of the molecule through a heteroatom. Non-limiting examples of suitable groups include 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 4-benzothiazolyl, 5-benzothiazolyl, 6-benzothiazolyl, 7-benzothiazolyl, purinyl, 2-benzimidazolyl, 4-indolyl, 5-indolyl, 6-indolyl, 7-indolyl, 1-isoquinolyl, 5-isoquinolyl, 2-quinoxalinyl, 5-quinoxalinyl, 2-quinolyl, 3-quinolyl, 4-quinolyl, 5-quinolyl, 6-quinolyl, 7-quinolyl or 8-quinolyl.

Preferred moieties for Ar¹ are phenyl, biphenyl, naphthyl, indolyl (which is 2,3-benzopyrrolyl), benzothiozolyl, and quinoyl. More preferred are phenyl, biphenyl, and naphthyl, even more preferred are phenyl and naphthyl, and most preferred is naphthyl. A preferred naphthyl is 2-naphthyl. When an aryl is monocyclic and has a substituent, it is preferably linked para to the substituent. When an aryl is fused bicyclic and it has a substituent, it is preferably linked meta to the atoms shared by both cycles.

Cyc is an aromatic, heteroaromatic, cyclic, or heterocyclic C5-10 hydrocarbon. Preferred examples of Cyc are C5-10 aryl, C6-10 alkylaryl, cyclopentyl, cyclohexyl, cycloheptyl, N-piperidyl, and N-methylate piperidyl. Within Cyc, preferred aryl is phenyl, imidazolyl, and thiophenyl, most preferably phenyl. within Cyc, preferred cyclic hydrocrabons are cyclohexyl, cyclopentyl, and cycloheptyl, most preferably cyclohexyl. Preferably Cyc is phenyl or cyclohexyl. When comprised in Cyc, a 5-membered ring is preferably connected to the remainder of general formula (1) at non-adjacent positions on the ring. When comprised in Cyc, a 6-membered ring is preferably connected to the remainder of general formula (1) at non-adjacent positions on the ring, most preferably para to one another. When comprised in Cyc, a 7-membered ring is preferably connected to the remainder of general formula (1) at non-adjacent positions on the ring, most preferably having at least 2 ring atoms separating the ring atoms that link to the remainder of general formula (1).

X is H, a protecting group, a chelator, a detectable label, a pharmaceutically active agent, an albumin-binding moiety, or a linker, wherein the linker is optionally attached to a chelator, a detectable label, a pharmaceutically active agent, or two or more of a chelator, a detectable label, and a pharmaceutically active agent. Because X is linked to a nitrogen atom, when X is a protecting group, definitions as for P⁵ preferably apply. Preferred protecting groups for amines are formed when X is benzyl, benzyl carbamate, benzoyl, tert-butyl carbamate, 9-fuorenylmethyl carbamate, tosyl, —C(phenyl)₃, trifluoroacetyl, acetyl, or optionally a phthalimide forming moiety, of which benzyl, benzyl carbamate, tert-butyl carbamate, and 9-fuorenylmethyl carbamate are more preferred, and tert-butyl carbamate is most preferred. Definitions for a chelator, a detectable label, a linker, and a pharmaceutically active agent are provided later herein. When X is H or a linker, the compound can be said to be a precursor.

Preferred albumin-binding moieties for X are 4-(4-methylphenyl)-butyryl (so the compound is a derivative of 4-para-tolylbutyric acid) and 2-(4-isobutylphenyl) propionyl (so the compound is a derivative of isobutylphenylpropionic acid, also known as ibuprofen). Albumin-binding moieties can help extend the circulation time of the compounds. Association with albumins per se is a known strategy for extending circulation time. Preferably the albumin-binding moiety is a human serum albumin binding moiety. Such moieties are generally small hydrocarbons with comprising a phenyl ring substituted with at least two carbon atoms.

Some embodiments provide the compound according to the invention, wherein
i) P¹, P², P³, and P⁴, are each independently H or a protecting group that is a C1-7 hydrocarbon; or
ii) P⁵ is a C2-8 acyl group, preferably comprising a C5-6 aromatic or heteroaromatic ring; or
iii) e¹ is 1; or e² is 1; or
iv) k¹ is 1; or k² is 1; or
v) i is 0; or j is 1; or
vi) h¹ is H; or h² is H; or h³ is H; or
vii) Ar¹ is naphthyl, phenyl, biphenyl, indolyl, benzothiazolyl, or quinoyl; or
viii) Cyc is a C5-10 aryl, a C6-10 alkylaryl, cyclopentyl, cyclohexyl, cycloheptyl, or piperidyl.

Preferably at least one of the above apply, more preferably at least two of the above apply, even more preferably at least three, still more preferably at least four, still more preferably at least five, still more preferably at least six, still more preferably at least seven, most preferably all eight of the above apply. In some embodiments, both i and ii apply. In some embodiments, i and ii and iii apply. In some embodiments, i and ii and iii and iv apply. In some embodiments each of i-v apply, in some embodiments each of i-vi apply, in some embodiments each of i-vii apply. In some embodiments all except i apply. In some embodiments all except ii apply. In some embodiments all except iii apply. In some embodiments all except iv apply. In some embodiments all except v apply. In some embodiments all except vi apply. In some embodiments all except vii apply. In some embodiments all except viii apply. In some embodiments at least i applies. In some embodiments at least ii applies. In some embodiments at least iii applies. In some embodiments at least iv applies. In some embodiments at least v applies. In some embodiments at least vi applies. In some embodiments at least vii applies. In some embodiments at least viii applies.

Some embodiments provide the compound according to the invention, wherein
i) P¹, P², P³, and P⁴, are each H or tert-butyl; or
ii) P⁵ is benzoyl, picolinyl, nicotinyl, or isonicotinyl; or
iii) e¹ and e² are 1; or
iv) k¹ and k² are 1; or
v) i is 0 and j is 1; or
vi) h¹, h² and h³ are H; or
vii) Ar¹ is naphthyl; or
viii) Cyc is phenyl, cyclopentyl, cyclohexyl, cycloheptyl, or piperidyl.

Preferably at least one of the above apply, more preferably at least two of the above apply, even more preferably at least three, still more preferably at least four, still more preferably at least five, still more preferably at least six, still more preferably at least seven, most preferably all eight of the above apply. In some embodiments, both i and ii apply. In some embodiments, i and ii and iii apply. In some embodiments, i and ii and iii and iv apply. In some embodiments each of i-v apply, in some embodiments each of i-vi apply, in some embodiments each of i-vii apply. In some embodiments all except i apply. In some embodiments all except ii apply. In some embodiments all except iii apply. In some embodiments all except iv apply. In some embodiments all except v apply. In some embodiments all except vi apply. In some embodiments all except vii apply. In some embodiments all except viii apply. In some embodiments at least i applies. In some embodiments at least ii applies. In some embodiments at least iii applies. In some embodiments at least iv applies. In some embodiments at least v applies. In some embodiments at least vi applies. In some embodiments at least vii applies. In some embodiments at least viii applies.

Some embodiments provide the compound according to the invention, wherein it is of general formula (1-L), (1-D), or (1-LL):

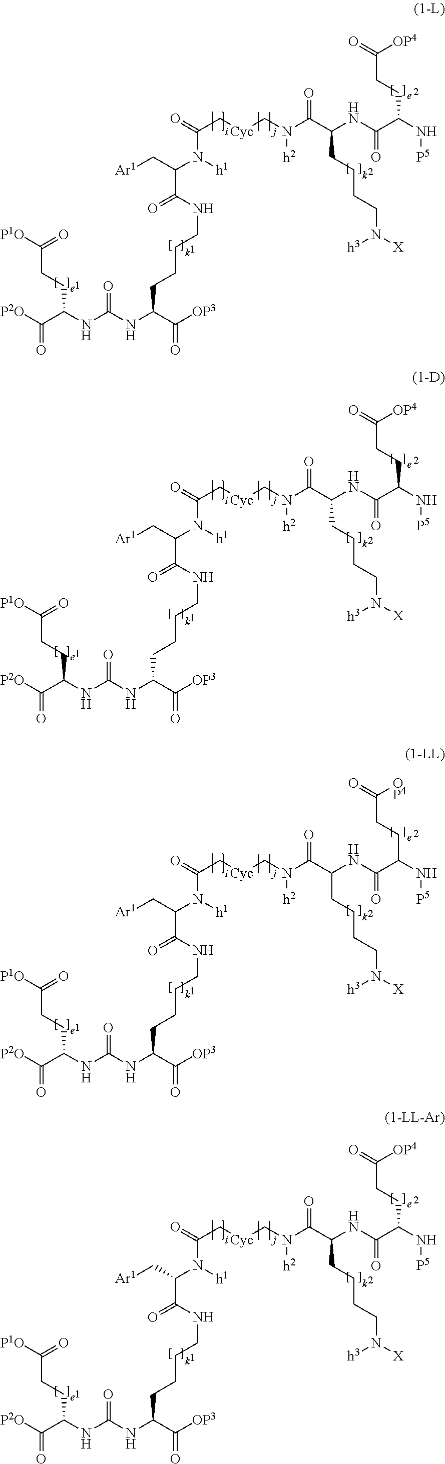

Of these, general formula (1-L) and (1-LL) and (1-LL-Ar) are preferred, and (1-L) and (1-LL-Ar) are more preferred, while (1-LL-Ar) is most preferred. Whenever a compound according to the invention is represented herein, it preferably has stereochemistry as in general formula (1-L) or (1-LL-Ar), most preferably (1-LL-Ar).

Other preferred compounds according to the invention are of general formula (2), (3), (4), (5), (6), or (7). Of these, (4), (5), (6), and (7) are preferred, (6) and (7) are more preferred, and (7) is most preferred. In (6) and (7), the moiety bearing Ar¹ forms a 3-(2-naphthyl)-L-alanine (Nal) residue. In (7) the moiety Cyc forms a (4-aminomethyl)benzoic acid (Amb) linker.

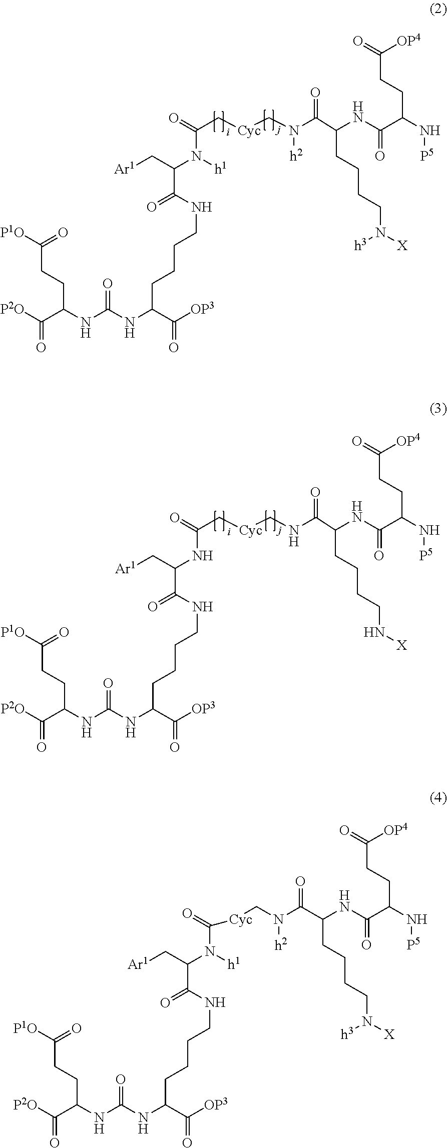

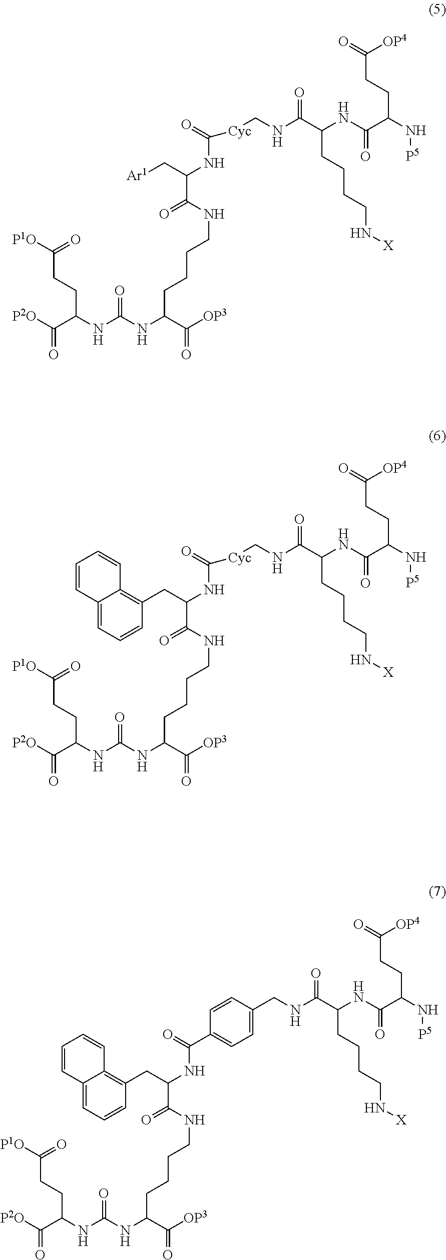

### The Compound as Agent for Imaging or Treatment

The invention also provides the compound according to the invention, wherein X is a chelator, a detectable label, or a linker, wherein the linker is optionally attached to a chelator, a detectable label, or both a chelator and a detectable label. In preferred embodiments X is a chelator. In preferred embodiments X is a chelator or a detectable label. In preferred embodiments X is linker that is attached to a chelator. In preferred embodiments X is linker that is attached to a detectable label. In highly preferred embodiments X is linker that is attached to both a chelator and a detectable label.

Chelators are known in the art (see for instance Price and Orvig, DOI: 10.1039/C3CS60304K), and are useful for associating the PSMA-ligand with radiolabels. Suitable chelators for a radiolabel are 1,4,7,10-tetraazacyclododecane-tetraacetic acid (DOTA) 1,4,7-triazacyclononane-triacetic acid (NOTA), triazacyclononane-phosphinate (TRAP), 1,4,8,11-tetraazacyclotetradecane-1,4,8, 11-tetraacetic acid (TETA), N,N′-bis[2-hydroxy-5-(carboxyethyl)benzyl] ethylenediamine-N,N′-diacetic acid (HBED-CC) and diethylenetriaminepentaacetic anhydride (DTPA) or its hydrolyzed form. Further chelators for radiolabels can be based on 2,2′,2″,2″-(Ethane-1,2-diyldinitrilo)tetraacetic acid (EDTA) or 1,4,8,11-Tetraazacyclotetradecane (cyclam) or 1,8-diamino-3,6,10,13,16,19-hexaazabicyclo[6.6.6] icosane (DiamSar). In preferred embodiments, chelators are selected from HYNIC (6-hydrazinonicotinic hydrazide), MAG3 (mercaptoacetyltriglycine), MAS3 (acetylmercaptoacetyltriserine), DTPA (diethylenetriaminepentaacetic acid), chelators for tricarbonyl radiolabels such as Tc99m-tricarbonyl (for instance L1, L8, L9, and L10 from Banerjee et al., DOI: 10.1021/jm400823w), DFO (deferoxamine), DFO* (see Patra et al., Chem Commun. 2014; 50:11523-5), TAFC (triacetylfusarinine C), FSC (Fusarinine C), HBED-CC, THP (Tris (hydroxypyridinone)), TRAP, DOTA, NODAGA (1,4,7-triazacyclononane, 1-glutaric acid-4,7-acetic acid), NOTA, DOTAGA (2,2′,2″-(10-(2,6-dioxotetrahydro-2H-pyran-3-yl)-1,4,7,10-tetraazacyclododecane-1,4,7-triyl)triacetic acid), CHX-A″-DTPA (such as [(R)-2-amino-3-(4-isothiocyanatophenyl) propyl]-trans-(S,S)-cyclohexane-1,2-diamine-pentaacetic acid), TCMC (such as 2-[(4-Isothiocyanatophenyl)methyl]-1,4,7,10-tetraazacyclododecane-1,4,7,10-tetraacetamide tetrahydrochloride), DO3AM (2-(4,7,10-tris(2-amino-2-oxoethyl)-1,4,7,10-tetraazacyclododecan-1-yl) acetic acid), NETA (({4-[2-(bis-carboxy-methylamino)-5-(4-nitrophenyl) pentyl]-7-carbo-xymethyl-[1,4,7]triazanonan-1-yl} acetic acid), CB-DO2A (4,10-bis (carboxymethyl)-1,4,7,10-tetraazabicyclo[5.5.2] tetradecane), TETA (1,4,8, 11-tetraazacyclotetradecane-1,4,8, 11-tetraacetic acid), DiamSar, NODA-MPAA (the 1,4,7-triazacyclononane-1,4-diacetate (NODA) motif with a methylphenylacetic acid (MPAA) backbone, see DOI: 10.1021/bc200175c), and RESCA (WO2016/065435). Highly preferred chelators are DOTA and NOTA, while DOTA is most preferred.

Preferred Tc-99m-chelators are HYNIC, MAG3, MAS3, DTPA, and chelators for tricarbonyl Tc-99m, more preferably HYNIC, MAG3, MAS3, and DTPA. Preferred Zr-89 chelators are DFO, DFO*, TAFC, and FSC. Preferred Ga-68 chelators are HBED-CC, TAFC, FSC, THP, TRAP, DOTA, NODAGA, and NOTA. Preferred In-111 chelators are DTPA, DOTA, NOTA, DOTAGA, and CHX-A″-DTPA. Preferred Pb-chelators are TCMC, DO3AM, and NETA. Preferred Cu chelators are DOTA, CB-DO2A, TETA, and Diamsar. Preferred AIF-18 chelators are NODA-MPAA, NOTA, and RESCA.

These chelators are able to result in very stable complexes of many metals or with radioactive isotopes of metals that are routinely used in radiology and nuclear medicine, such as stable Gd, ¹¹¹In, ⁹⁰Y, ^{99m}Tc, ¹⁷⁷Lu, ⁶⁸Ga. The chelated metal itself does not need to be the actual radiolabel. For example, 18F can be effectively conjugated as an axial ligand to chelated aluminum, wherein the aluminum can be of conventional natural abundance (McBride et al., EJNMMI Research 2013 3:36, DOI: 10.1186/2191-219X-3-36 and DOI: 10.2967/jnumed. 108.060418.) Gadolinium can also be of conventional natural abundance for MRI applications. In preferred embodiments a conjugate according to the invention is a conjugated targeting peptide according to the invention, wherein the targeting peptide is conjugated to a chelator for a radiolabel, wherein said chelator is complexed with a radiolabel. In the context of this application, preferred radiolabels are selected from the group consisting of ¹¹¹In, ⁹⁰Y, ^{99m}Tc, ¹⁷⁷Lu, ⁶⁸Ga, ¹⁸F, ²¹²Pb, ²²⁵Ac, ²¹²Bi, ²¹¹As, ⁸⁹Zr, ⁶⁴Cu, ⁶⁷Cu, ⁴⁴Sc, ⁴⁷Sc, ¹⁴⁹T, ¹⁵²Tb, ¹⁵⁵Tb, ¹⁶¹Tb, ²⁰³Pb, and ²²⁷Th, more preferred radiolabels are ¹¹¹In, ⁹⁰Y, ^{99m}Tc, ¹⁷⁷Lu, ⁶⁸Ga, and ¹⁸F, the most preferred radiolabels are ¹¹¹In, ²¹²Pb, ¹⁸F, and ^{99m}Tc. Chelating non-radioactive metal can be useful while studying pharmacodynamic properties of a conjugate according to the invention, as it obviates the need for precautions that are normally required for working with radiolabels. In preferred embodiments a compound according to the invention comprises a chelator for a radiolabel, wherein said chelator is complexed with a non-radioactive isotope. Such a non-radioactive isotope is sometimes referred to as a non-radioactive radiolabel or a cold radiolabel and it can be either depleted atoms or natural abundance atoms. A preferred compound according to the invention is a compound according to the invention wherein it is conjugated to a chelator for a radiolabel, preferably DOTA or NOTA.

A detectable label can be a fluorophore, a chromophore, a radiolabel, a specific isotope, a diagnostic marker, or a hapten, wherein a fluorophore is preferably fluorescein or its derivatives such as FITC or Tokyo green, ASP (preferably 4-(4-(didecylamino) styryl)-N-methylpyridinium iodide), rhodamine, Cyanine5 (also known as Cy5), Cyanine5.5 (also known as Cy5.5), Cyanine7 (also known as Cy7), sulfoCyanine7 (also known as sulfoCy7), Cyanine7.5 (also known as Cy7.5), IRDye 700DX, IRDye 800CW, IRDye 800ZW, Alexa660, Alexa680, Alexa700, Alexa750, Alexa790, DyLight 755, DyLight 800 Fluoprobes 752, FluoProbes 782, calcein, any other Alexa-label, any other Cyanine-label, and sulfonated or otherwise modified variants of any of these fluorophores.

Other preferred detectable labels are chromophores suitable for use as an optoacoustic chromophore. Photoacoustic (PA) imaging (PAI), or optoacoustic imaging, is a hybrid imaging modality that merges optical illumination and ultrasound (US) detection (for a review see Amalina Binte Ebrahim Attia et al., doi: 10.1016/j.pacs.2019.100144 or Upputuri and Pramanik DOI: 10.1002/wnan.1618). Suitable chromophores can be metallic nanoparticles, carbon-based nanomaterials, quantum dots, organic small molecules, semiconducting polymer nanoparticles, and so on. Preferred examples are indocyanine green and methylene blue. In preferred embodiments, a detectable label can be a fluorophore or a chromophore suitable for use as an optoacoustic chromophore.

Preferred embodiments provide the compound according to the invention, wherein the chelator is 1,4,7,10-tetraazacyclododecane-tetraacetic acid (DOTA) 1,4,7-triazacyclononane-triacetic acid (NOTA), triazacyclononane-phosphinate (TRAP), 1,4,8,11-tetraazacyclotetradecane-1,4,8, 11-tetraacetic acid (TETA), N,N′-bis[2-hydroxy-5-(carboxyethyl)benzyl] ethylenediamine-N,N′-diacetic acid (HBED-CC), or diethylenetriaminepentaacetic anhydride (DTPA); and/or wherein the detectable label is a fluorophore, a chromophore, a radiolabel, a specific isotope, a diagnostic marker, or a hapten, wherein a fluorophore is preferably fluorescein or its derivatives such as FITC or Tokyo green, ASP (preferably 4-(4-(didecylamino) styryl)-N-methylpyridinium iodide), rhodamine, Cyanine5 (also known as Cy5), Cyanine5.5 (also known as Cy5.5), Cyanine7 (also known as Cy7), sulfoCyanine7 (also known as sulfoCy7), Cyanine7.5 (also known as Cy7.5), IRDye 700DX, IRDye 800CW, IRDye 800ZW, Alexa660, Alexa680, Alexa700, Alexa750, Alexa790, DyLight 755, DyLight 800 Fluoprobes 752, FluoProbes 782, calcein, any other Alexa-label, any other Cyanine-label, and sulfonated or otherwise modified variants of any of these fluorophores.

Preferred embodiments provide the compound according to the invention, wherein the compound comprises a detectable label. Other preferred embodiments provide the compound according to the invention, wherein the compound comprises a chelator. Highly preferred embodiments provide the compound according to the invention, wherein the compound comprises both a chelator and a detectable label. In such embodiments a linker is generally also comprised, most likely a bifunctional linker such as lysine.

In some embodiments, X is a pharmaceutically active agent. The nature of this agent is not critical to the invention, and a skilled person can select a suitable pharmaceutically active agent to link to the compound according to the invention. Due to the PSMA-targeting capacity of the compounds according to the invention, it is preferred that the pharmaceutically active agent be a cytostatic, a chemotherapeutic drug, or an agent that increases the efficacy of a cytostatic or of a chemotherapeutic drug.

Such pharmaceutically active agents include, but are not limited to, chemotherapeutic drugs. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma and calicheamicin omega); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholmo-doxorubicm, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-II); topoisomerase inhibitor RFS 2000; difluorometlhylornithine (DMFO); retinoids such as retinoic acid; capecitabine; gefitinib and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LYI 17018, onapristone, and toremifene; aromatase inhibitors that inhibit the enzyme aromatase, which regulates estrogen production in the adrenal glands, such as, for example, 4 (5)-imidazoles, aminoglutethimide, megestrol acetate, exemestane, formestanie, fadrozole, vorozole, letrozole, and anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in aberrant cell proliferation, such as, for example, PKC-α, Raf and H-Ras; ribozymes such as a VEGF expression inhibitor and a HER2 expression inhibitor; vaccines such as gene therapy vaccines and pharmaceutically acceptable salts, acids or derivatives of any of the above. A suitable RNR inhibitor is selected from the group consisting of gemcitabine, hydroxyurea, clolar, clofarabine, and triapine. A suitable AURKB inhibitor is selected from the group consisting of: AZD1152, VX-680, MLN8054, MLN8237, PHA680632, PH739358, Hesperidin, ZM447439, JNJ770621, SU6668, CCT129202, AT9283, MP529, SNS314, R763, ENMD2076, XL228, TTP687, PF03814735 and CYC116. Another suitable anticancer drug is gefitinib.

X can also be a linker. The linker is optionally attached to a chelator, a detectable label, a pharmaceutically active agent, or two or more of a chelator, a detectable label, and a pharmaceutically active agent. More preferably, when the linker is attached to a further moiety, it is attached to a detectable label, a chelator, or both to a detectable label and a chelator. The linker provides a functional group for further functionalisation, which can be as defined for X⁴ later herein.

Multiple PSMA-targeting tracers have been developed, including PSMA-617 and more recently PSMA-1007 (WO2017/054907). PSMA-617 and PSMA-1007 are potent agents for radio-imaging and therapy of PSMA-positive cancers and both have been translated to clinical applications. Both tracers demonstrated high affinity for PSMA, as well as high internalization as a result of their excellent fit in the substrate binding pocket of PSMA.

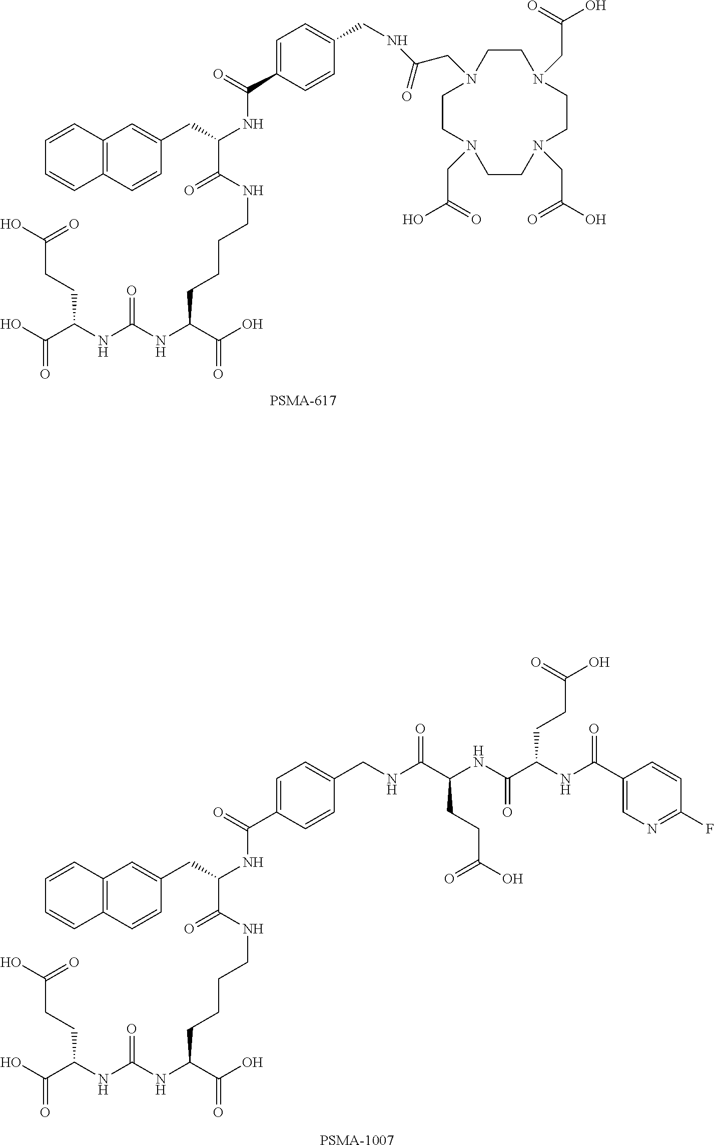

To be able to conjugate one or more functional elements to the tracer, the inventors modified the peptide linker of PSMA-1007 by substitution of one of the glutamate residues into a lysine residue (see FIG. 1). The inventors surprisingly found that this lysine allows conjugation of further moieties, without adversely impacting binding. For some embodiments binding was even improved.

For instance, for compounds of general formula (1-ahx), X is aminohexanoic acid (or the acyl radical thereof, as will be clear to a skilled reader). Such a compound can be further derivatised to yield compounds of general formula (1-ahx2).

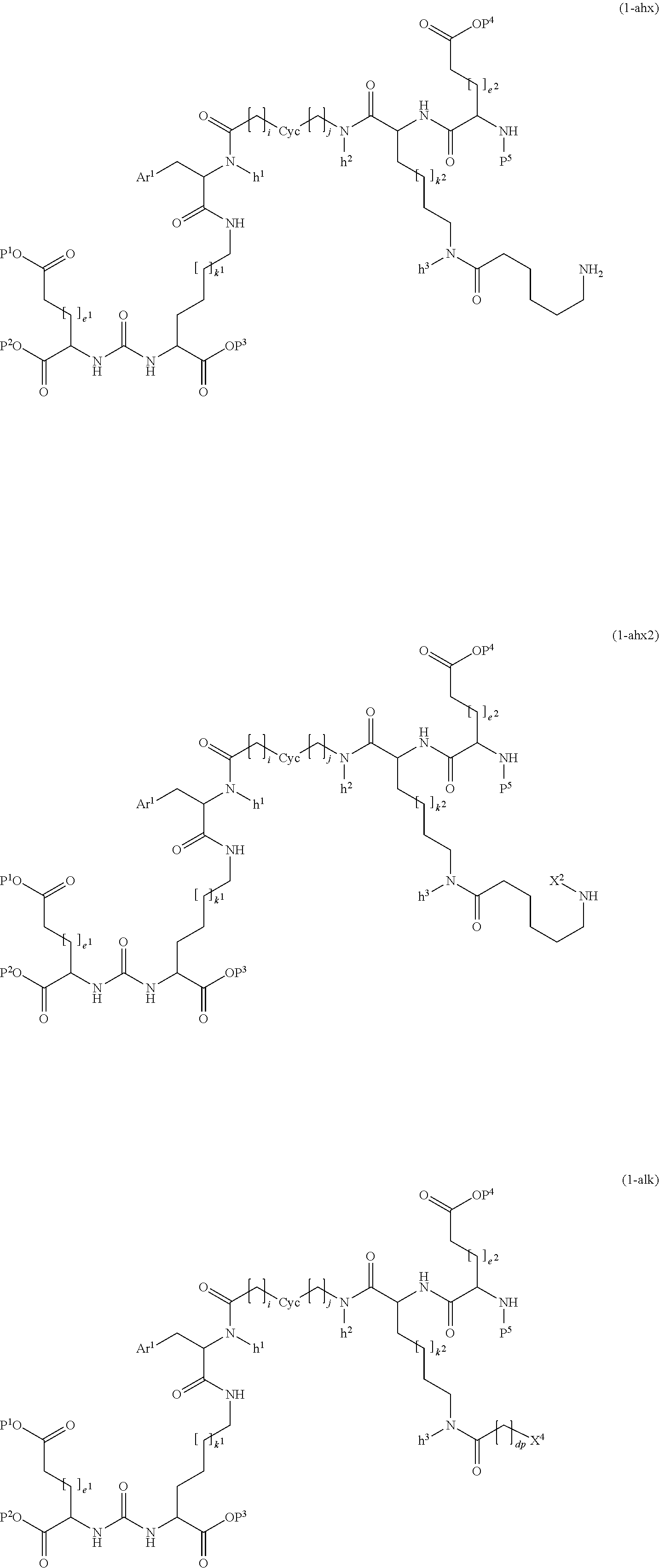

Herein, X² is a chelator, a detectable label, a pharmaceutically active agent, or H; preferably X² is a chelator, a detectable label, or H; more preferably X² is a chelator or a detectable label.

A preferred linker is a bifunctional alkyl linker, of which an example is shown in general formula (1-alk). Herein, dp refers to the degree of polymerisation of a linear alkyl linker, and it can be any positive integer up to 50. Most preferably it is 1, 2, 3, 4, 5, 6, 7, 8, 9, or 10; more preferably it is 1, 2, 3, 4, 5, or 6. Even more preferably it is 1, 2, 3, or 4. X⁴ is defined later herein. Preferably, one or two methylene units governed by dp are substituted with an additional instance of X⁴ that is independently selected. More preferably one such methylene unit is substituted with an additional instance of X⁴ that is independently selected.

In preferred embodiments, the linker provides two such further groups. For instance, for compounds of general formula (1-K), lysine is used as the linker. Such a compound can be further derivatised to yield compounds of general formula (1-K2), wherein each of X² and X³ is independently a chelator, a detectable label, a pharmaceutically active agent, or H; preferably a chelator, a detectable label, or H; more preferably a chelator or a detectable label. In preferred embodiments, when X² is a chelator, X³ is not a chelator. In preferred embodiments, when X² is a detectable label, X³ is not a detectable label.

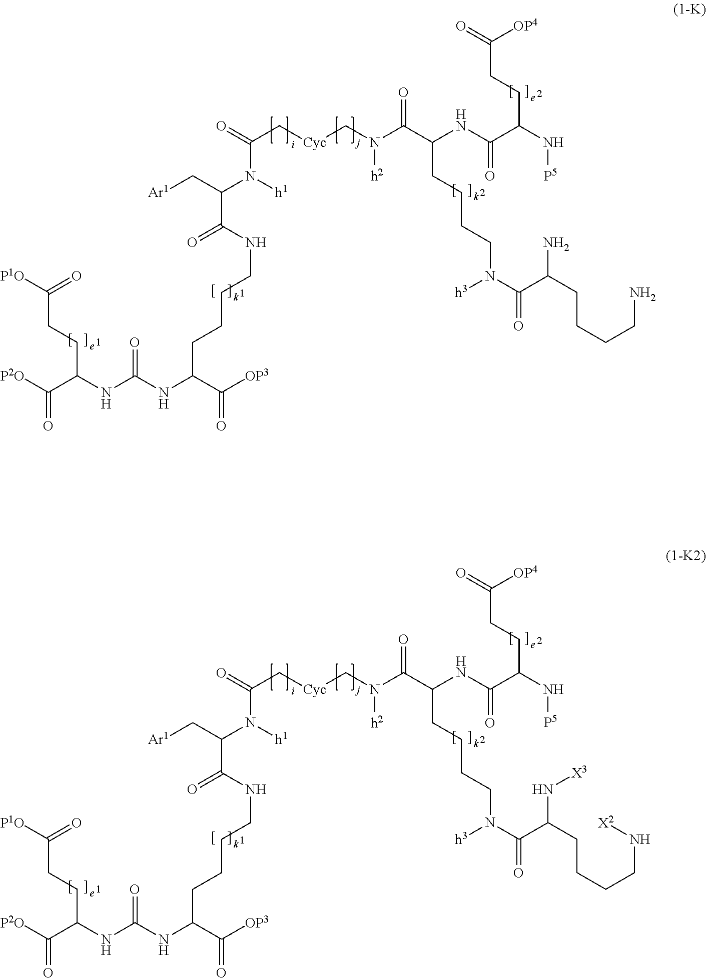

Preferred embodiments provide the compound according to the invention, wherein the linker comprises an amino acid, an oligo (ethylene glycol), or a C2-12 hydrocarbon, wherein the linker comprises at least one functional group for further modification, wherein the linker preferably comprises two functional groups for further modification. A hydrocarbon has been defined earlier, and a preferred hydrocarbon is the acyl radical of aminohexanoic acid, wherein the amine of aminohexanoic acid is optionally protected with a protecting group.

The term “amino acid” refers to naturally occurring and non-natural amino acids, as well as amino acid analogs and amino acid mimetics that function in a manner similar to the naturally occurring amino acids. Naturally amino acids are the 20 common amino acids in their D- or L-form (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refers to compounds that have the same basic chemical structure as a naturally occurring amino acid, by way of example only, an ex-carbon that is bound to a hydrogen, a carboxyl group, an amino group, and an R group. Such analogs may have modified R groups (by way of example, norleucine) or may have modified peptide backbones, while still retaining the same basic chemical structure as a naturally occurring amino acid. Non-limiting examples of amino acid analogs include homoserine, norleucine, methionine sulfoxide, methionine methyl sulfonium. Amino acids may be referred to herein by either their name, their commonly known three letter symbols or by the one-letter symbols recommended by the IUPAC-IUB Biochemical Nomenclature Commission. A “non-natural amino acid” refers to an amino acid that is not one of the 20 common amino acids or pyrolysine or selenocysteine. Other terms that may be used synonymously with the term “non-natural amino acid” is “non-naturally encoded amino acid,” “unnatural amino acid,” “nonnaturally-occuning amino acid” or “artificial amino acid. The term “non-natural amino acid” includes, but is not limited to, amino acids which occur by modification of a naturally encoded amino acid in their backbone or side chains. In some embodiments the non-natural amino acid comprises a carbonyl group, an acetyl group, an aminooxy group, a hydrazine group, a hydrazide group, a semicarbazide group, an azide group or an alkyne group. Preferred amino acids are glutamic acid, aspartic acid, serine, threonine, ornithine, lysine, cysteine, and tyrosine; more preferred are glutamic acid, aspartic acid, ornithine, and lysine; even more preferred are ornithine and lysine, and lysine is most preferred.

An oligo (ethylene glycol) is a moiety wherein the—O—CH₂—CH₂— motif is repeated at least once. Oligo (ethylene glycol) linkers are widely available from commercial suppliers, and can be obtained with myriad functional groups on either terminus of the oligomer. An example of a compound of general formula (1) wherein X is an oligo (ethylene glycol) linker is shown in general formula (1-oegm), wherein a repeating oligo (ethylene glycol) (OEG) is separated from a carboxylic acid or X⁴ via a methylene moiety (M).

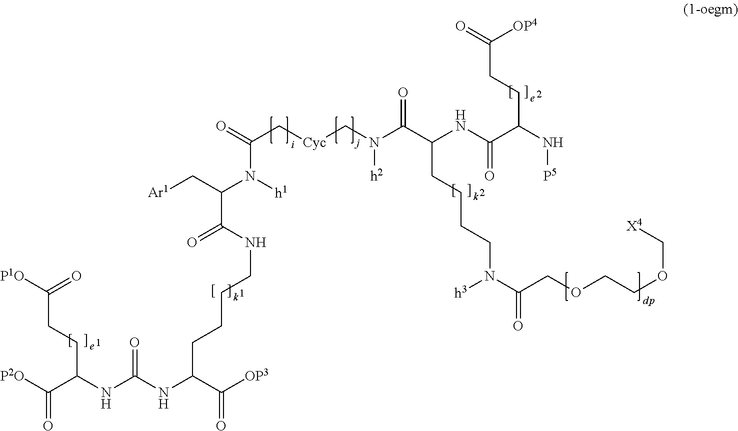

Herein, dp refers to the degree of polymerisation of the OEG, and it can be any positive integer up to 50. Most preferably it is 1, 2, 3, 4, 5, 6, 7, 8, 9, or 10; more preferably it is 1, 2, 3, 4, 5, or 6. Even more preferably it is 2, 3, or 4.

X⁴ is H, a functional group for further modification that is optionally preceded by a C1-6alkyl, a protected functional group that is optionally preceded by a C1-6alkyl, a chelator, a detectable label, or a pharmaceutically active substance. Preferably, X⁴ is H, a functional group for further modification that is optionally preceded by a C1-6alkyl, a protected functional group that is optionally preceded by a C1-6alkyl, a chelator, or a detectable label. More preferably X⁴ is H, a functional group for further modification, a protected functional group, a chelator, or a detectable label. Preferred functional groups for further modification are —OH, —COOH, —SH, —NH₂, —N₃, and -alkyne. More preferred are —COOH, —SH, —N₃, and —NH₂, and —NH₂ is most preferred. Suitable protecting groups have been described elsewhere herein; preferred protected functional for groups X⁴ are —C(═O)O—P⁶ wherein P⁶ is as defined for P¹, and —NH—P⁷ wherein P⁷ is as defined for P⁵.

### Additional Examples of Compounds

Some embodiments provide the compound according to the invention, wherein the compound is selected from compounds of general formula (1) wherein:
1) P¹, P², P³, and P⁴, are H; P⁵ is nicotinyl; e¹ and e² are 1; k¹ and k² are 1; i is 0; j is 1; h¹, h², and h³ are H; Ar¹ is naphthyl; Cyc is phenyl; X is H;
2) P¹, P², P³, and P⁴, are H; P⁵ is nicotinyl; e¹ and e² are 1; k¹ and k² are 1; i is 0; j is 1; h¹, h², and h³ are H; Ar¹ is naphthyl; Cyc is phenyl; X is lysine (—C(═O)C(NH₂)(CH₂)₄NH₂);
3) P¹, P², P³, and P⁴, are H; P⁵ is nicotinyl; e¹ and e² are 1; k¹ and k² are 1; i is 0; j is 1; h¹, h², and h³ are H; Ar¹ is naphthyl; Cyc is phenyl; X is α-DOTA-lysine (—C(═O)C(NH[DOTA])(CH₂)₄NH₂);
4) P¹, P², P³, and P⁴, are H; P⁵ is nicotinyl; e¹ and e² are 1; k¹ and k² are 1; i is 0; j is 1; h¹, h², and h³ are H; Ar¹ is naphthyl; Cyc is phenyl; X is &-IRDye700DX-lysine (—C(═O)C(NH₂)(CH₂)₄NH[IRDye700DX]);
5) P¹, P², P³, and P⁴, are H; P⁵ is nicotinyl; e¹ and e² are 1; k¹ and k² are 1; i is 0; j is 1; h¹, h², and h³ are H; Ar¹ is naphthyl; Cyc is phenyl; X is α-DOTA-&-IRDye700DX-lysine (—C(═O)C(NH [DOTA])(CH₂)₄NH [IRDye700DX]);
6) P¹, P², P³, and P⁴, are H; P⁵ is benzoyl; e¹ and e² are 1; k¹ and k² are 1; i is 0; j is 1; h¹, h², and h³ are H; Ar¹ is naphthyl; Cyc is phenyl; X is H;
7) P¹, P², P³, and P⁴, are H; P⁵ is benzoyl; e¹ and e² are 1; k¹ and k² are 1; i is 0; j is 1; h¹, h², and h³ are H; Ar¹ is naphthyl; Cyc is phenyl; X is lysine (—C(═O)C(NH₂)(CH₂)₄NH₂);
8) P¹, P², P³, and P⁴, are H; P⁵ is benzoyl; e¹ and e² are 1; k¹ and k² are 1; i is 0; j is 1; h¹, h², and h³ are H; Ar¹ is naphthyl; Cyc is phenyl; X is α-DOTA-lysine (—C(═O)C(NH [DOTA])(CH₂)₄NH₂);
9) P¹, P², P³, and P⁴, are H; P⁵ is benzoyl; e¹ and e² are 1; k¹ and k² are 1; i is 0; j is 1; h¹, h², and h³ are H; Ar¹ is naphthyl; Cyc is phenyl; X is &-IRDye700DX-lysine (—C(═O)C(NH₂)(CH₂)₄NH [IRDye700DX]);
10) P¹, P², P³, and P⁴, are H; P⁵ is benzoyl; e¹ and e² are 1; k¹ and k² are 1; i is 0; j is 1; h¹, h², and h³ are H; Ar¹ is naphthyl; Cyc is phenyl; X is α-DOTA-&-IRDye700DX-lysine (—C(═O)C(NH [DOTA])(CH₂)₄NH [IRDye700DX]);
11) P¹, P², P³, and P⁴, are H; P⁵ is nicotinyl; e¹ and e² are 1; k¹ and k² are 1; i is 0; j is 1; h¹, h², and h³ are H; Ar¹ is naphthyl; Cyc is cyclohexyl; X is H;
12) P¹, P², P³, and P⁴, are H; P⁵ is nicotinyl; e¹ and e² are 1; k¹ and k² are 1; i is 0; j is 1; h¹, h², and h³ are H; Ar¹ is naphthyl; Cyc is cyclohexyl; X is lysine (—C(═O)C(NH₂)(CH₂)₄NH₂);
13) P¹, P², P³, and P⁴, are H; P⁵ is nicotinyl; e¹ and e² are 1; k¹ and k² are 1; i is 0; j is 1; h¹, h², and h³ are H; Ar¹ is naphthyl; Cyc is cyclohexyl; X is α-DOTA-lysine (—C(═O)C(NH [DOTA])(CH₂)₄NH₂);
14) P¹, P², P³, and P⁴, are H; P⁵ is nicotinyl; e¹ and e² are 1; k¹ and k² are 1; i is 0; j is 1; h¹, h², and h³ are H; Ar¹ is naphthyl; Cyc is cyclohexyl; X is &-IRDye700DX-lysine (—C(═O)C(NH₂)(CH₂)₄NH [IRDye700DX]);
15) P¹, P², P³, and P⁴, are H; P⁵ is nicotinyl; e¹ and e² are 1; k¹ and k² are 1; i is 0; j is 1; h¹, h², and h³ are H; Ar¹ is naphthyl; Cyc is cyclohexyl; X is α-DOTA-&-IRDye700DX-lysine (—C(═O)C(NH [DOTA])(CH₂)₄NH [IRDye700DX]);
16) P¹, P², P³, and P⁴, are H; P⁵ is benzoyl; e¹ and e² are 1; k¹ and k² are 1; i is 0; j is 1; h¹, h², and h³ are H; Ar¹ is naphthyl; Cyc is cyclohexyl; X is H;
17) P¹, P², P³, and P⁴, are H; P⁵ is benzoyl; e¹ and e² are 1; k¹ and k² are 1; i is 0; j is 1; h¹, h², and h³ are H; Ar¹ is naphthyl; Cyc is cyclohexyl; X is lysine (—C(═O)C(NH₂)(CH₂)₄NH₂);
18) P¹, P², P³, and P⁴, are H; P⁵ is benzoyl; e¹ and e² are 1; k¹ and k² are 1; i is 0; j is 1; h¹, h², and h³ are H; Ar¹ is naphthyl; Cyc is cyclohexyl; X is α-DOTA-lysine (—C(═O)C(NH [DOTA])(CH₂)₄NH₂);
19) P¹, P², P³, and P⁴, are H; P⁵ is benzoyl; e¹ and e² are 1; k¹ and k² are 1; i is 0; j is 1; h¹, h², and h³ are H; Ar¹ is naphthyl; Cyc is cyclohexyl; X is &-IRDye700DX-lysine (—C(═O)C(NH₂)(CH₂)₄NH [IRDye700DX]); and
20) P¹, P², P³, and P⁴, are H; P⁵ is benzoyl; e¹ and e² are 1; k¹ and k² are 1; i is 0; j is 1; h¹, h², and h³ are H; Ar¹ is naphthyl; Cyc is cyclohexyl; X is α-DOTA-s-IRDye700DX-lysine (—C(═O)C(NH [DOTA])(CH₂)₄NH [IRDye700DX]);

wherein preferably the compound is of general formula (1-L). Preferred herein are 1, 6, 11, and 16, more preferably 1, which are useful as precursors. Also preferred are 2, 7, 12, and 17, more preferably 2, which are useful precursors for further dual labelling. Also preferred are 3, 8, 13, and 18, more preferably 3, which are useful for further radiolabelling. Also preferred are 4, 9, 14, and 19, more preferably 4, which are useful for phototherapy. Highly preferred are 5, 10, 15, and 20, more preferably 5, which are useful dual-labelled compounds, or for forming such. A highly preferred set of compounds is 1, 2, 3, 4, and 5.

Further preferred compounds according to the invention are shown below. K15 and K19 are particularly preferred.

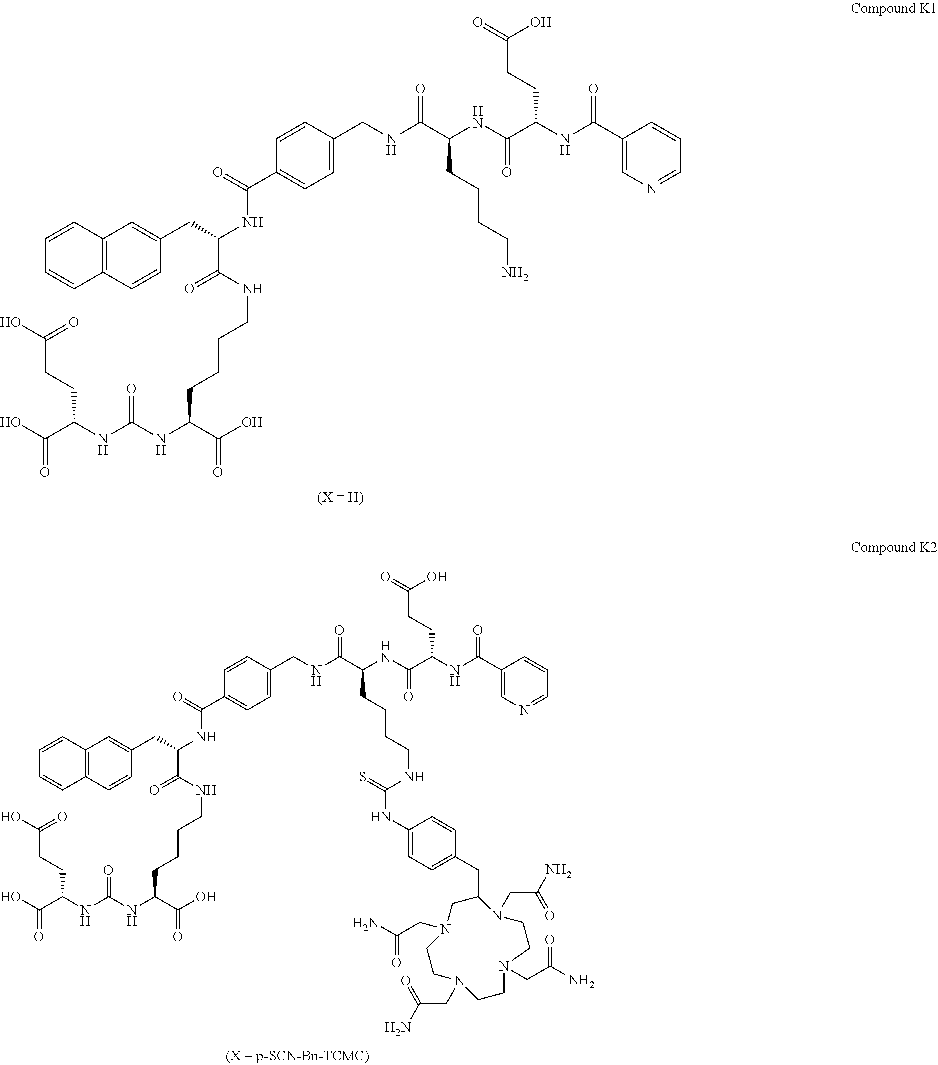 [SMILES: NCCCC[C@H](NC(=O)[C@H](CCC(=O)O)NC(=O)c1cccnc1)C(=O)NCc1ccc(C(=O)N[C@@H](Cc2ccc3ccccc3c2)C(=O)NCCCC[C@H](NC(=O)N[C@@H](CCC(=O)O)C(=O)O)C(=O)O)cc1 ; NC(=O)CN1CCN(CC(N)=O)CCN(CC(N)=O)C(Cc2ccc(NC(=S)NCCCC[C@H](NC(=O)[C@H](CCC(=O)O)NC(=O)c3cccnc3)C(=O)NCc3ccc(C(=O)N[C@@H](Cc4ccc5ccccc5c4)C(=O)NCCCC[C@H](NC(=O)N[C@@H](CCC(=O)O)C(=O)O)C(=O)O)cc3)cc2)CN(CC(N)=O)CC1]

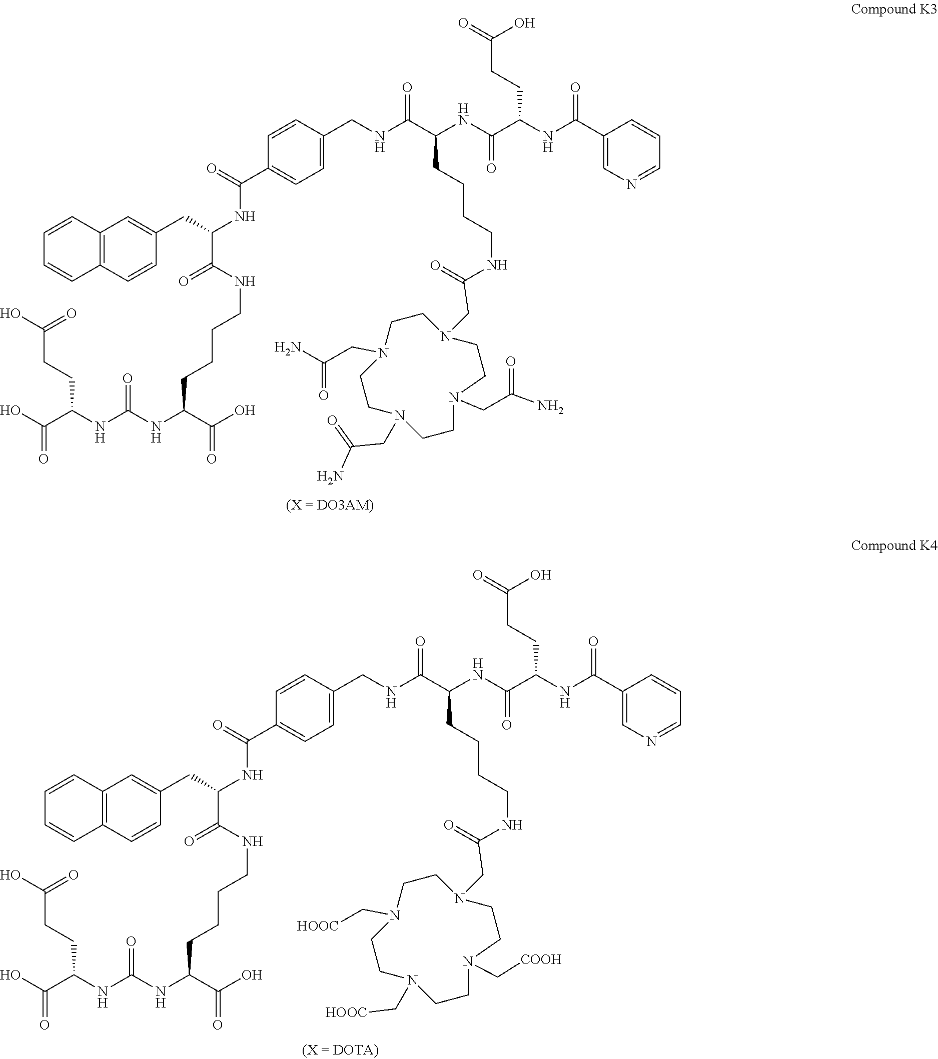 [SMILES: NC(=O)CN1CCN(CC(N)=O)CCN(CC(=O)NCCCC[C@H](NC(=O)[C@H](CCC(=O)O)NC(=O)c2cccnc2)C(=O)NCc2ccc(C(=O)N[C@@H](Cc3ccc4ccccc4c3)C(=O)NCCCC[C@H](NC(=O)N[C@@H](CCC(=O)O)C(=O)O)C(=O)O)cc2)CCN(CC(N)=O)CC1 ; O=C(O)CC[C@H](NC(=O)N[C@@H](CCCCNC(=O)[C@H](Cc1ccc2ccccc2c1)NC(=O)c1ccc(CNC(=O)[C@H](CCCCNC(=O)CN2CCN(CC(=O)O)CCN(CC(=O)O)CCN(CC(=O)O)CC2)NC(=O)[C@H](CCC(=O)O)NC(=O)c2cccnc2)cc1)C(=O)O)C(=O)O]

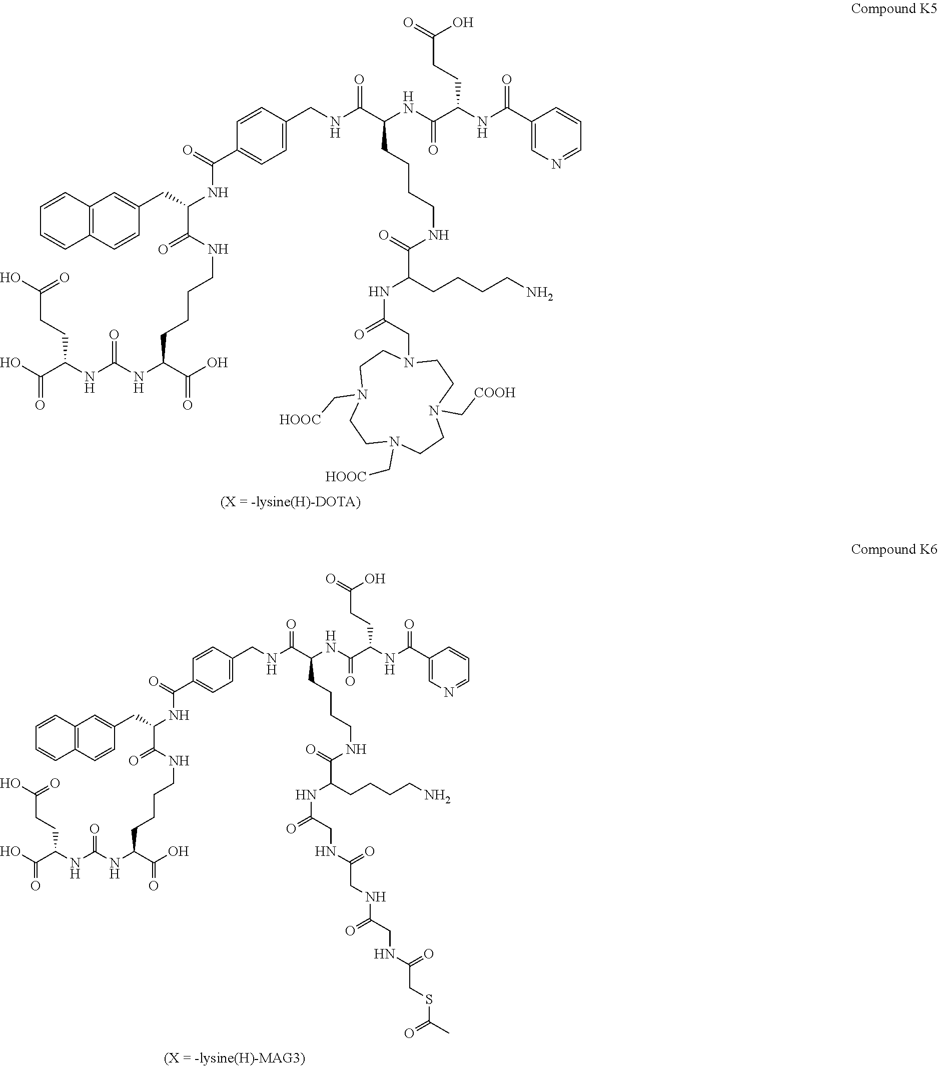 [SMILES: NCCCCC(NC(=O)CN1CCN(CC(=O)O)CCN(CC(=O)O)CCN(CC(=O)O)CC1)C(=O)NCCCC[C@H](NC(=O)[C@H](CCC(=O)O)NC(=O)c1cccnc1)C(=O)NCc1ccc(C(=O)N[C@@H](Cc2ccc3ccccc3c2)C(=O)NCCCC[C@H](NC(=O)N[C@@H](CCC(=O)O)C(=O)O)C(=O)O)cc1 ; CC(=O)SCC(=O)NCC(=O)NCC(=O)NCC(=O)NC(CCCCN)C(=O)NCCCC[C@H](NC(=O)[C@H](CCC(=O)O)NC(=O)c1cccnc1)C(=O)NCc1ccc(C(=O)N[C@@H](Cc2ccc3ccccc3c2)C(=O)NCCCC[C@H](NC(=O)N[C@@H](CCC(=O)O)C(=O)O)C(=O)O)cc1]

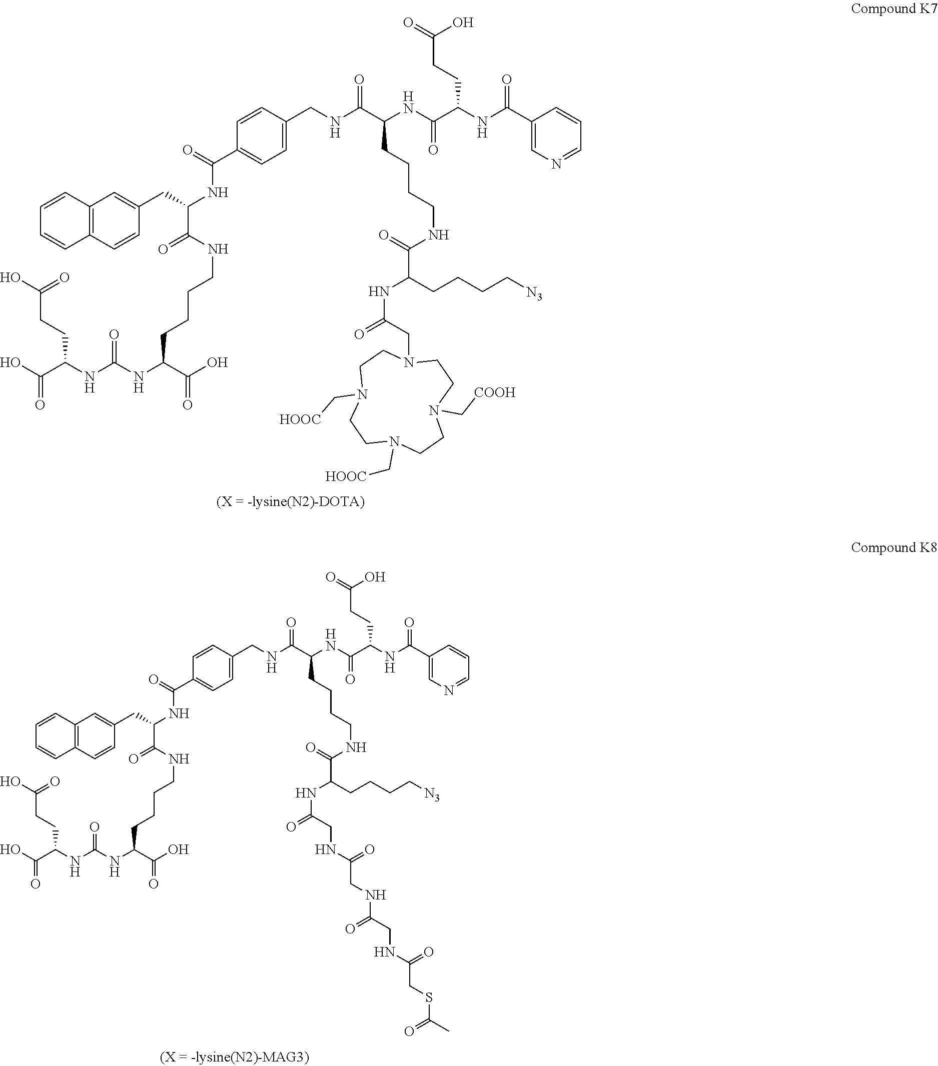 [SMILES: [N-]=[N+]=NCCCCC(NC(=O)CN1CCN(CC(=O)O)CCN(CC(=O)O)CCN(CC(=O)O)CC1)C(=O)NCCCC[C@H](NC(=O)[C@H](CCC(=O)O)NC(=O)c1cccnc1)C(=O)NCc1ccc(C(=O)N[C@@H](Cc2ccc3ccccc3c2)C(=O)NCCCC[C@H](NC(=O)N[C@@H](CCC(=O)O)C(=O)O)C(=O)O)cc1 ; CC(=O)SCC(=O)NCC(=O)NCC(=O)NCC(=O)NC(CCCCN=[N+]=[N-])C(=O)NCCCC[C@H](NC(=O)[C@H](CCC(=O)O)NC(=O)c1cccnc1)C(=O)NCc1ccc(C(=O)N[C@@H](Cc2ccc3ccccc3c2)C(=O)NCCCC[C@H](NC(=O)N[C@@H](CCC(=O)O)C(=O)O)C(=O)O)cc1]

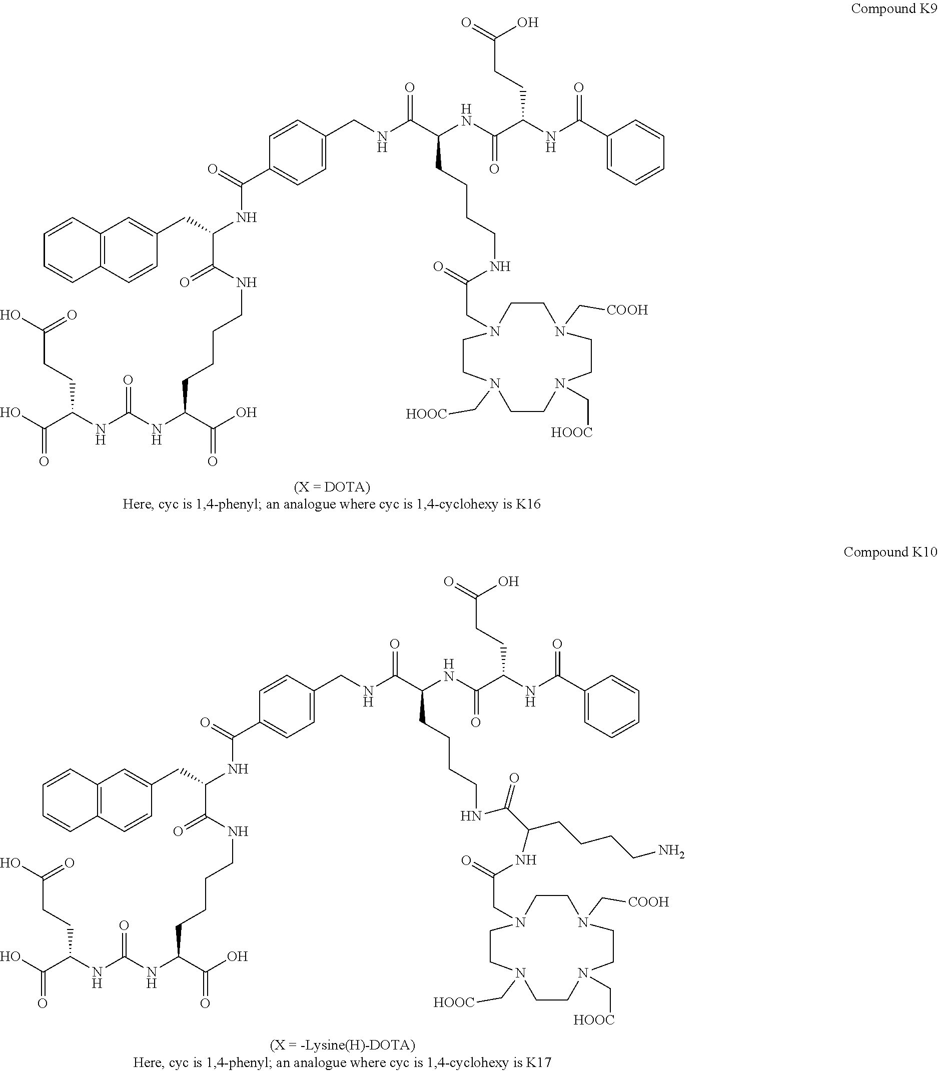 [SMILES: O=C(O)CC[C@H](NC(=O)N[C@@H](CCCCNC(=O)[C@H](Cc1ccc2ccccc2c1)NC(=O)c1ccc(CNC(=O)[C@H](CCCCNC(=O)CN2CCN(CC(=O)O)CCN(CC(=O)O)CCN(CC(=O)O)CC2)NC(=O)[C@H](CCC(=O)O)NC(=O)c2ccccc2)cc1)C(=O)O)C(=O)O ; NCCCCC(NC(=O)CN1CCN(CC(=O)O)CCN(CC(=O)O)CCN(CC(=O)O)CC1)C(=O)NCCCC[C@H](NC(=O)[C@H](CCC(=O)O)NC(=O)c1ccccc1)C(=O)NCc1ccc(C(=O)N[C@@H](Cc2ccc3ccccc3c2)C(=O)NCCCC[C@H](NC(=O)N[C@@H](CCC(=O)O)C(=O)O)C(=O)O)cc1]

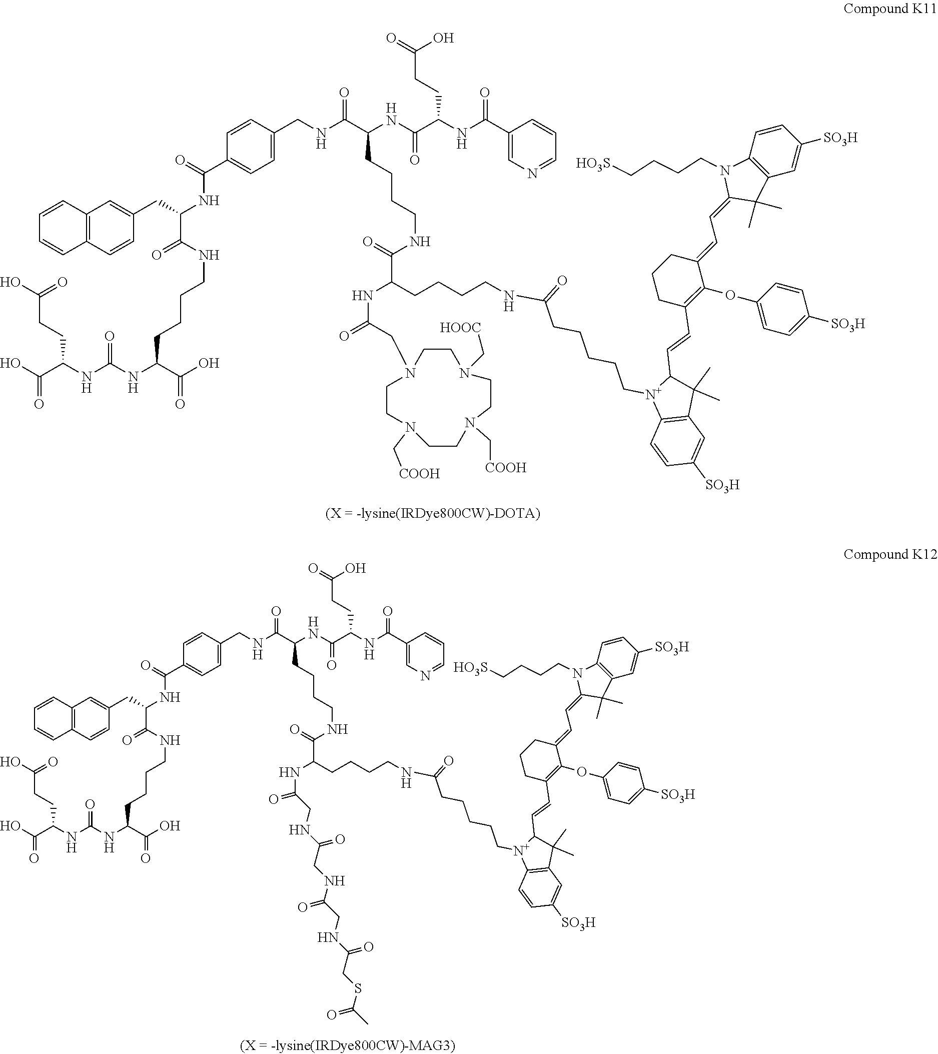

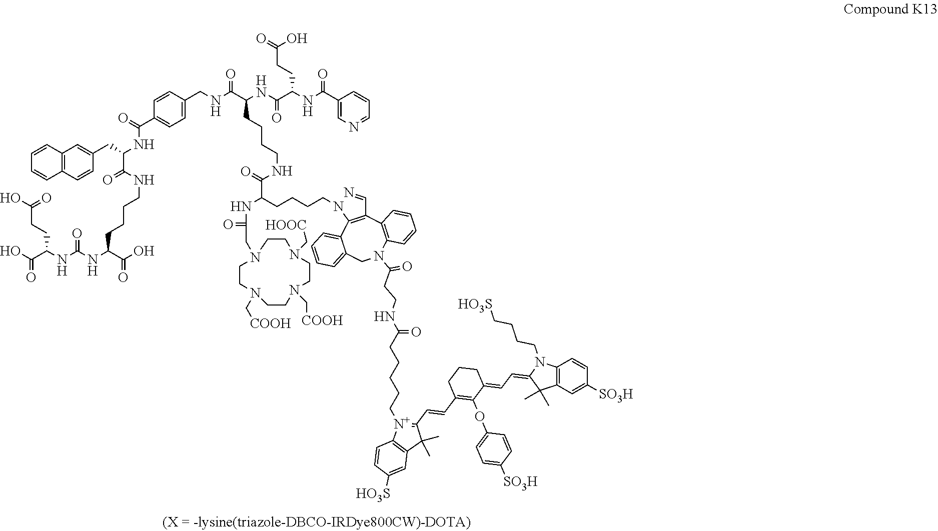 [SMILES: CC1(C)C(/C=C/C2=C(Oc3ccc(S(=O)(=O)O)cc3)C(=C/C=C3/N(CCCCS(=O)(=O)O)c4ccc(S(=O)(=O)O)cc4C3(C)C)/CCC2)=[N+](CCCCCC(=O)NCCC(=O)N2Cc3ccccc3-c3c(cnn3CCCCC(NC(=O)CN3CCN(CC(=O)O)CCN(CC(=O)O)CCN(CC(=O)O)CC3)C(=O)NCCCC[C@H](NC(=O)[C@H](CCC(=O)O)NC(=O)c3cccnc3)C(=O)NCc3ccc(C(=O)N[C@@H](Cc4ccc5ccccc5c4)C(=O)NCCCC[C@H](NC(=O)N[C@@H](CCC(=O)O)C(=O)O)C(=O)O)cc3)-c3ccccc32)c2ccc(S(=O)(=O)O)cc21]

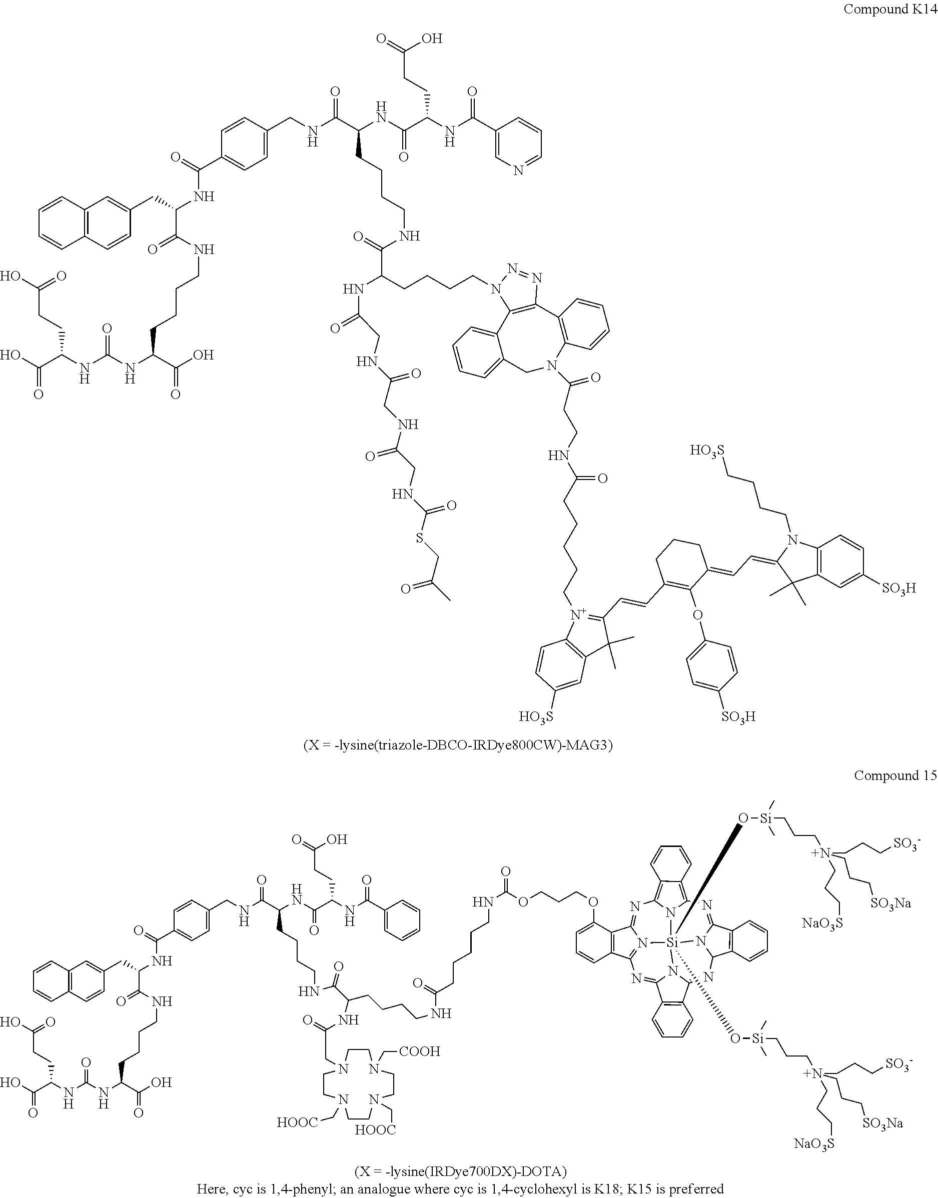

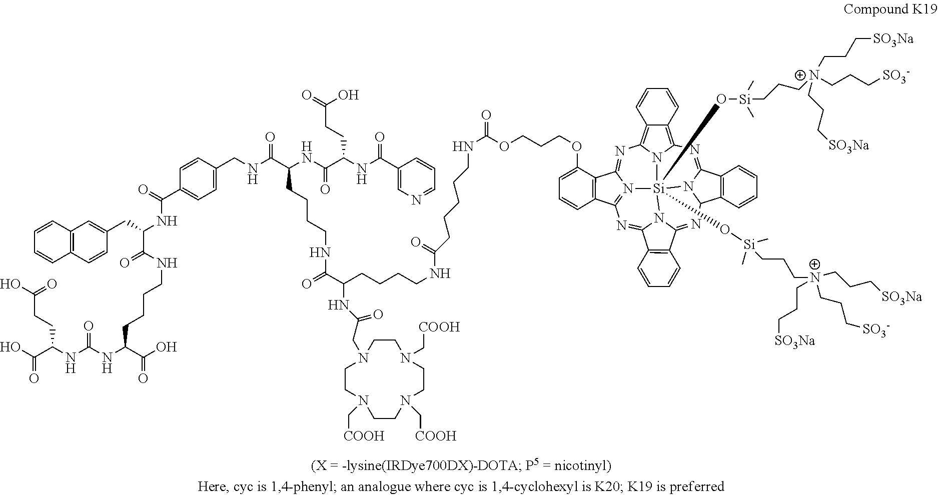

### Compositions

The invention also provides a composition comprising a compound according to the invention and a pharmaceutically acceptable excipient. Such a composition is referred to herein as a composition according to the invention. Preferably, such a composition is formulated as a pharmaceutical composition. A preferred excipient is water, preferably purified water, more preferably ultrapure water. In other embodiments the water is a pharmacologically acceptable buffer such as saline, buffered saline, or more preferably phosphate buffered saline. A composition preferably has a physiologically acceptable pH, more preferably in the range of 6 to 8. Further preferred excipients are adjuvants, binders, desiccants, or diluents. Further preferred compositions additionally comprise additional medicaments for treating cancer or for treating conditions as described later herein. Preferred additional medicaments in this regards are chemotherapeutic agents, immunotherapeutic agents, or steroids for the treatment of cancer.

Compositions according to the invention preferably contain a diagnostically or therapeutically effective amount of the compound according to the invention. This is optionally combined with organic or inorganic solid or liquid, or with pharmaceutically acceptable carriers which are suited for the intended administration and which interact with the active ingredients without drawbacks.

### Use of the Compounds

The compound according to the invention can be used as a medicament. The medicament is preferably for treating a cancer and/or a metastasis thereof, preferably wherein the cancer is a prostate cancer or a salivary gland cancer, more preferably a prostate cancer. The invention is particularly suited when the medicament is for treatment or diagnosis of PSMA-expressing cancers. In the context of this invention, a preferred cancer is prostate cancer, a salivary gland cancer, neuroblastoma, glioma, leukaemia, lung cancer, bladder cancer, renal cancer, pancreatic cancer, adenocarcinoma, or epithelial cancer, and more preferably prostate cancer or a salivary gland cancer. Preferred examples of epithelial cancer are colorectal cancer, breast cancer, head and neck cancer, and prostate cancer. The treatment, prevention or delay of cancer is preferably the treatment, prevention or delay of cancer metastasis. Such compounds for use are referred to herein as compounds for use according to the invention. A composition according to the invention can also be used as a medicament, as described in this section. Such a composition is preferably a pharmaceutical composition, and can be referred to as a composition for use according to the invention.

To create a probe visible for the eye of a medical practitioner, and having high penetration depth in human tissue, fluorescence guided surgery (FGS) and radio guided surgery (RGS) can be combined into a single compound according to the invention, wherein the probe contains a fluorescent label as well as a chelating moiety that can carry a radiolabel. Preoperatively the location of the tumour tissue can be determined with the help of the radionuclide by PET or SPECT imaging. During the surgical procedure, the medical practitioner can rely on the signal generated by the radionuclide to be guided to the tumour tissue, until the fluorescent label becomes visible. The fluorescent label precisely evaluates resection margins once the surgical field is exposed to the surface, which can help the surgeon in removing all tumour cells. The fluorescent label can also allow the compound for use according to the invention to be used for subsequent or parallel photodynamic therapy.

Due to the radiolabel and fluorescent label linked to a targeting moiety, the compounds according to the invention are suitable for diagnosis. This diagnosis can be prior to treatment, but according to the unique nature of the compounds it can also be during treatment, or (immediately) after treatment. Accordingly, embodiments provide the compound for use according to the invention, wherein the medicament is for imaging, diagnosing, and/or treating a cancer and/or a metastasis thereof. Such a method is preferably for achieving a diagnosis via two parameters, such as via a radiolabel and via a fluorescent label.

The compounds for use according to the invention, and the compositions for use according to the invention, as described herein, can be very suitably used in a method of imaging, diagnosing, or treating cancer in a subject in need thereof, the method comprising the step of administering a compound or composition according to the invention to the subject.

A “subject” includes an animal, such as a human, monkey, cow, horse, cat, dog, mouse, or rat. The animal can be a mammal such as a non-primate and a primate (e.g., monkey and human). In one embodiment, a subject is a human being. In other embodiments the subject is not a human, such as a mouse or a rat.

In general, the compounds and compositions according to the invention may be administered orally or via a parenteral route, usually injection or infusion. A “parenteral administration route” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticluare, subcapsular, subarachnoid, intraspinal and intrasternal injection and infusion.

The dosage of the compounds according to the invention is determined by the physician on the basis of subject-specific parameters, such as age, weight, sex, severity of the disease, etc. Corresponding to the kind of administration, the medicament is suitably formulated, e.g. in the form of solutions or suspensions, simple tablets or dragees, hard or soft gelatine capsules, suppositories, ovules, preparations for injection, which are prepared according to common galenic methods.

When solutions for infusion or injection are used, they are preferably aqueous solutions or suspensions, it being possible to produce them prior to use, e.g. from lyophilized preparations which contain the active substance as such or together with a carrier, such as mannitol, lactose, glucose, albumin and the like. The ready-made solutions are sterilized and, where appropriate, mixed with excipients, e.g. with preservatives, stabilizers, emulsifiers, solubilizers, buffers and/or salts for regulating the osmotic pressure. The sterilization can be obtained by sterile filtration using filters having a small pore size according to which the composition can be lyophilized, where appropriate. Small amounts of antibiotics can also be added to ensure the maintenance of sterility.

The phrases “effective amount” or “therapeutically-effective amount” as used herein means that amount of a compound, material, or composition comprising a compound of the invention, or other active ingredient which is effective for producing some desired therapeutic effect in at least a sub-population of cells in an animal at a reasonable benefit/risk ratio applicable to any medical treatment. A therapeutically effective amount with respect to a compound of the invention means that amount of therapeutic agent alone, or in combination with other therapies, that provides a therapeutic benefit in the treatment of prevention of a disease. Used in connection with a compound according to the invention, the term can encompass an amount that improves overall therapy, reduces or avoids symptoms or causes of disease, or enhances the therapeutic efficacy of or synergies with another therapeutic agent.

As used herein, the terms “treating” or “treatment” is intended to encompass also diagnosis, prophylaxis, prevention, therapy and cure. The terms “prevent”, “preventing,” and “prevention” refer to the prevention of the onset, recurrence, or spread of the disease in a patient resulting from the administration of a prophylactic or therapeutic agent. As noted above, compounds according general formula I are suitable for use as radioimaging agents or as therapeutics for the treatment of rapidly proliferating cells, for example, PSMA-expressing prostate cancer cells. According to the present invention they can be called “radiopharmaceuticals”.

Preferred imaging methods are positron emission tomography (PET) or single photon emission computed tomography (SPECT). Accordingly, in one embodiment, a pharmaceutical composition is provided including a compound according to the invention, a salt, solvate, stereoisomer, or tautomer thereof, and a pharmaceutically acceptable carrier. Accordingly, a pharmaceutical composition is provided, which is suitable for in vivo imaging and radiotherapy. Suitable pharmaceutical compositions may contain the compound according to the invention in an amount sufficient for imaging, together with a pharmaceutically acceptable radiological vehicle. The radiological vehicle should be suitable for injection or aspiration, such as human serum albumin; aqueous buffer solutions, e.g., tris (hydromethyl) aminomethane (and its salts), phosphate, citrate, bicarbonate, etc; sterile water physiological saline; and balanced ionic solutions containing chloride and or dicarbonate salts or normal blood plasma cautions such as calcium potassium, sodium and magnesium.

The concentration of the imaging agent or the therapeutic agent in the radiological vehicle should be sufficient to provide satisfactory imaging. For example, when using an aqueous solution, the dosage is about 1.0 to 100 millicuries, or 37 MBq to 7.4 GBq, preferably to 1GBq. The actual dose administered to a patient for imaging or therapeutic purposes, however, is determined by the physician administering treatment. The imaging agent or therapeutic agent should be administered so as to remain in the patient for about 1 hour to 10 days, although both longer and shorter time periods are acceptable. Therefore, convenient ampoules/vials containing 1 to 20 mL, preferably 1 to 10 mL of aqueous solution may be prepared. Imaging may be carried out in the normal manner, for example by injecting a sufficient amount of the imaging composition to provide adequate imaging and then scanning with a suitable imaging or scanning machine, such as a tomograph or gamma camera. In certain embodiments, a method of imaging a region in a subject includes the steps of: (i) administering to a subject a diagnostically effective amount of a compound labeled with a radionuclide; exposing a region of the patient to the scanning device; and (ii) obtaining an image of the region of the subject.

The amount of the compound according to the present invention, or its salt, solvate, stereoisomer, or tautomer that is administered to a patient depends on several physiological factors that are routinely used by the physician, including the nature of imaging to be carried out, tissue to be targeted for imaging or therapy and the body weight and medical history of the patient to be imaged or treated using a radiopharmaceutical. Specifically, the cell proliferative disease or disorder to be treated or imaged using a compound, pharmaceutical composition or radiopharmaceutical in accordance with this invention is a cancer, for example, prostate cancer and/or prostate cancer metastasis in e.g. lung, liver, kidney, bones, brain, spinal cord, bladder, etc.

### General Definitions

When a structural formula or chemical name is understood by the skilled person to have chiral centers, yet no chirality is indicated, for each chiral center individual reference is made to all three of either the racemic mixture (having any enantiomeric excess), the pure R enantiomer, and the pure S enantiomer. Whenever a fragment of a molecule, often referred to as a moiety, is represented, a dotted or wavy line indicates which bond links it to the entirety of the molecule; alternately, an asterisk (*) indicates where the represented moiety is linked to the rest of the molecule. This asterisk does not imply an atom, and neither does a bond that is crossed by a dotted or wavy line convey information about which atom is at the non-moiety side of the bond. All this is known in the art.

Compounds and compounds for use provided in this invention can be optionally substituted. Suitable optional substitutions are replacement of —H by a halogen. Preferred halogens are F, Cl, Br, and I. Further suitable optional substitutions are substitution of one or more —H by —NH₂, —OH, ═O, alkyl, alkoxy, haloalkyl, haloalkoxy, alkene, haloalkene, alkyne, haloalkyn, and cycloalkyl. Alkyl groups have the general formula CₙH₂ₙ₊₁ and may alternately be linear or branched. Unsubstituted alkyl groups may also contain a cyclic moiety, and thus have the concomitant general formula CₙH₂ₙ₋₁. Optionally, the alkyl groups are substituted by one or more substituents further specified in this document. Examples of alkyl groups include methyl, ethyl, propyl, 2-propyl, t-butyl, 1-hexyl, 1-dodecyl, etc. Throughout this application, the valency of atoms should always be fulfilled, and H can be added or removed as required.

Unless stated otherwise, —H may optionally be replaced by one or more substituents independently selected from the group consisting of C₁-C₁₂ alkyl groups, C₂-C₁₂ alkenyl groups, C₂-C₁₂ alkynyl groups, C₃-C₁₂ cycloalkyl groups, C₅-C₁₂ cycloalkenyl groups, C₈-C₁₂ cycloalkynyl groups, C₁-C₁₂ alkoxy groups, C₂-C₁₂ alkenyloxy groups, C₂-C₁₂ alkynyloxy groups, C₃-C₁₂ cycloalkyloxy groups, halogens, amino groups, oxo and silyl groups, wherein the silyl groups can be represented by the formula (R²)₃Si—, wherein R² is independently selected from the group consisting of C₁-C₁₂ alkyl groups, C₂-C₁₂ alkenyl groups, C₂-C₁₂ alkynyl groups, C₃-C₁₂ cycloalkyl groups, C₁-C₁₂ alkoxy groups, C₂-C₁₂ alkenyloxy groups, C₂-C₁₂ alkynyloxy groups and C₃-C₁₂ cycloalkyloxy groups, wherein the alkyl groups, alkenyl groups, alkynyl groups, cycloalkyl groups, alkoxy groups, alkenyloxy groups, alkynyloxy groups and cycloalkyloxy groups are optionally substituted, the alkyl groups, the alkoxy groups, the cycloalkyl groups and the cycloalkoxy groups being optionally interrupted by one of more hetero-atoms selected from the group consisting of O, N and S. Preferably, these optional substitutions comprise no more than twenty atoms, more preferably no more than fifteen atoms.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37° C. (from about 20° C. to about 40° C.), and a suitable concentration of buffer salts or other components. It is understood that charge is often associated with equilibrium. A moiety that is said to carry or bear a charge is a moiety that will be found in a state where it bears or carries such a charge more often than that it does not bear or carry such a charge. As such, an atom that is indicated in this disclosure to be charged could be non-charged under specific conditions, and a neutral moiety could be charged under specific conditions, as is understood by a person skilled in the art.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

The use of a compound or composition as a medicament as described in this document can also be interpreted as the use of said compound or composition in the manufacture of a medicament. Similarly, whenever a compound or composition is used for as a medicament, it can also be used for the manufacture of a medicament, or in a method.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”. The word “about” or “approximately” when used in association with a numerical value (e.g. about 10) preferably means that the value may be the given value (of 10) more or less 1% of the value.

All patent and literature references cited in the present specification are hereby incorporated by reference in their entirety.

In the context of this invention, a cell or a sample can be a cell or a sample from a sample obtained from a subject. Such an obtained sample can be a sample that has been previously obtained from a subject. Such a sample can be obtained from a human subject. Such a sample can be obtained from a non-human subject.

### DESCRIPTION OF DRAWINGS

FIG. 1: comparison of PSMA-1007 from WO2017054907 (top) and the compounds of the present invention (bottom). Fluor from PSMA-1007 was omitted in the precursor according to the invention that is shown in this figure.

FIG. 2: percentage of γ-emission counts remaining after incubation of ¹¹¹In-labelled ligands with different chelators with PSMA-expressing LS174T cells. K4, K3, and K2 (compounds according to the invention where X is DOTA, DO3AM, or TCMC chelator, respectively) were tested. Light grey bars represent the total signal caused by ligand binding to the cells, while dark grey bars represent the signal caused by nonspecific binding as determined by blocking PSMA with PMPA.

FIG. 3A: tumor uptake for [¹¹¹In]In-DOTA (GA)-IRDye700DX-PSMA multimodal ligands. Biodistribution as determined after dissection of three 111In-labeled multimodal ligands (0.3 nmol, 10 MBq/mouse, 2 h p.i., n=4/group). Biodistribution was determined in mice bearing subcutaneous LS174T-PSMA and LS174T wildtype xenografts. Data is expressed as % ID/g±SD, ** indicates p<0.01, *** indicates p<0.001.

FIG. 3B: resulting tumor-to-organ ratios as for FIG. 3A.

FIG. 4A: μSPECT/CT and fluorescence images of multimodal PSMA-ligands. Representative same scale μSPECT/CT scans of mice with s.c. LS174T-PSMA (right) and wildtype LS174T (left) tumors after i.v. injection of 111In-labeled multimodal ligands (0.3 nmol, 10 MBq/mouse, 2 h p.i.).

FIG. 4B: fluorescence images as for FIG. 4A.

FIG. 5: tPDT efficacy of multimodal ligands and control in vitro. Cell viability of LS174T-PSMA (PSMA+) and LS174T wildtype (PSMA-) cells following incubation with 30 nM of compounds according to the invention or reference compounds after either a 100 J/cm² radiant exposure or no light exposure (dark). *** indicates p<0.001.

FIG. 6: Binding assay of 17c (comprising DO3AM), 17a (comprising DOTA), and 17b (comprising TCMC) on PSMA-transfected LS 174T cells. Non-specific binding was determined by blocking with an excess 2-PMPA (50 μg).

FIG. 7: Membrane bound and Internalization kinetics of 19a-c in PSMA-transfected LS174T cells. Non-specific binding was determined by blocking with an excess 2-PMPA (50 μg).

FIG. 8: Membrane bound and Internalization kinetics of 19a and 19b in PSMA-transfected LS174T cells, with and without addition of BSA. Non-specific binding was determined by blocking with an excess 2-PMPA (50 μg).

FIG. 9: membrane binding and internalization kinetics of K11, K12, K13, and K14 in LS174T PSMA-positive and negative cells. Non-specific binding was determined by blocking with an excess of 2-PMPA (50 μg). PSMA-617 was added as a positive control (Ref.) Using ^{99m}Tc and ¹¹¹In as indicated.

FIG. 10A: biodistribution as determined after dissection of ^{99m}Tc K14 (3 MBq/mouse) and ¹¹¹In K13 (10 MBq/mouse) 2, 4 and 24 hours p.i. (0.3 nmol, n=5/group). Biodistribution was determined in mice bearing subcutaneous LS174T-PSMA and LS174T wildtype xenografts. Data is expressed as % ID/g±SD, ** indicates p<0.01.

FIG. 10B: representative μSPECT/CT scans and fluorescence images of mice with s.c. LS174T-PSMA (left) and wildtype LS174T (right) tumors after i.v. injection of ^{99m}Tc K14 (3 MBq/mouse) and ¹¹¹In K13 (10 MBq/mouse) 2, 4 and 24 hours p.i. Epifluorescence scale is in 10⁷ and represents Rad. Eff. [p/sec/cm²/sr]/[μW/cm²].

FIG. 11A: in vivo comparison of K11. K12. K13. And K14. Biodistribution as determined after dissection of four ¹¹¹In-(10 MBq/mouse) or ^{99m}Tc-labeled (15 MBq/mouse) ligands and positive control PSMA-617 (0.3 nmol, 2 h p.i., n=5/group). Biodistribution was determined in mice bearing subcutaneous LS174T-PSMA and LS174T wildtype xenografts. Data is expressed as % ID/g±SD, * indicates p<0.05, ** indicates p<0.01, *** indicates p<0.001.

FIG. 11B: as 11A, showing resulting tumor-to-organ ratios.

FIG. 12A: All ligands clearly visualize PSMA-positive tumors using both μSPECT/CT and fluorescence imaging. Representative μSPECT/CT scans of mice with s.c. LS174T-PSMA (right) and wildtype LS174T (left) tumors after i.v. injection of ¹¹¹In-(10 MBq/mouse, K13, K11) or ^{99m}Tc-labeled (15 MBq/mouse, K14, K12) ligands (0.3 nmol, 2 h p.i.).

FIG. 12B: as for 12A showing fluorescence images. Epifluorescence scale is in 10⁷ and represents Rad. Eff. [p/sec/cm²/sr]/[μW/cm²].

FIG. 13A: Multimodal fluorescence and μSPECT/CT imaging of intraperitoneal PSMA-positive tumors using ¹¹¹In K11. Mouse with several intraperitoneal LS174T-PSMA tumors located at different depths in the peritoneal cavity. Same scale NIRF images of mouse with several intraperitoneal tumors after i.v. injection of ¹¹¹In-labeled K11 (0.3 nmol, 10 MBq/mouse, 2 h p.i.). Epifluorescence scale is in 10⁷ and represents Rad. Eff. [p/sec/cm²/sr]/[μW/cm²].

FIG. 13B: NIRF image of removed tumors from 13A.

FIG. 13C: corresponding μSPECT/CT images in supine and left lateral positions of mouse with several intraperitoneal tumors (arrows).

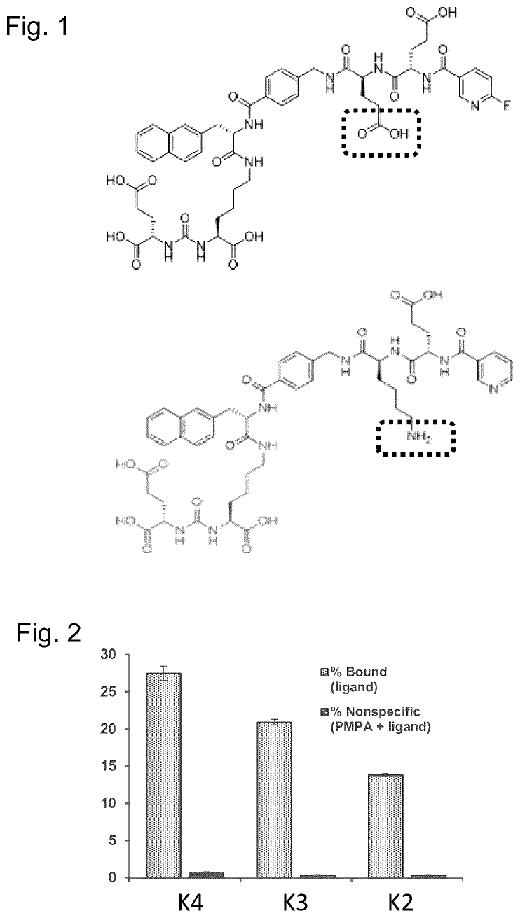

### EXAMPLES

### Example 1—Synthesis

### 1.1 General Procedures

All reactions have been carried out at room temperature unless stated otherwise. Purchased reagents were used without further purification. Mass spectra of peptides dissolved in H₂O with 50% MeCN (v/v) were recorded on a Thermo Finnigan LCQ Advantage Max system by ionization mode ESI (electrospray). For ligand-IRDye800CW conjugates, mass spectra were recorded on a Bruker Microflex LRF Maldi-tof system by ionization mode matrix-assisted laser desorption.

### 1.1.1 High Performance Liquid Chromatography (HPLC)

Analytical HPLC spectra of peptides dissolved in H₂O with 50% MeCN (v/v) were recorded on a Shimadzu LC-20A Prominence system. Peptides without fluorescent dye IRDye800CW were separated on a Gemini-Nx C18 column, 150×3 mm, particle size 3 μm, pore size 110 Å (Phenomenex, Torrance, California, U.S.A.) and eluted in aqueous solution containing 0.1% TFA (v/v) with increasing linear gradient of MeCN containing 0.1% TFA (v/V) (5-100%, 1-40 min., flow 0.4 mL/min). Peptides conjugated to IRDye800CW were separated on column and eluted in an aqueous triethylamine acetic acid buffer pH 7 with increasing linear gradient of methanol (5%-100%, 1-40 min., flow 0.4 mL/min).

All compounds were purified on a Shimadzu dual-pump LC-20A Prominence system (Shimadzu, 's Hertogenbosch, The Netherlands) equipped with a C18 Gemini-NX column, 150×10 mm, particle size 10 μm (Phenomenex, Utrecht, The Netherlands), applying a gradient of 20-70% methanol in triethylammonium acetate buffer (10 mM, pH 7) for all IRDye containing compounds or a gradient of 5-100% acetonitrile in water (0.1% TFA) for all others.

### 1.1.2 Fmoc Deprotection

For removal of Fmoc groups from the peptide, the resin was agitated with piperidine (20% in DMF, 10 mL per g resin) for 20 minutes. The resin was washed with DMF (3× with 10 mL per g resin) and CH₂Cl₂ (3× with 10 mL per g resin) after which deprotection was confirmed by a positive result in a dry Kaiser test. If no positive result was obtained, Fmoc deprotection was repeated.

### 1.1.3 Resin Loading Determination

For determination of peptide loading on the resin, piperidine (20% in DMF, 3.00 mL) was added an exact amount (˜2 mg) of resin. The suspension was agitated for 15 min after which the filtrate was collected and diluted 5× with DMF. The absorbance at 301 nm was measured and used to calculate the resin loading as described for conventional procedures in literature.

### 1.1.4 Kaiser Test

For a dry Kaiser test a few resin beads from resin (washed 3× with DMF, 3× with CH₂Cl₂) were separated and 3 droplets were added of respectively ninhydrin (0.3 M in EtOH), phenol (43 M in EtOH) and KCN (0.2 mM in pyridine). The mixture was heated in a boiling water bath for 1 min. The test was considered positive when the colour of the beads and solution was black to blue. Colourless to yellow beads and solution indicated a negative result. For a wet Kaiser test a few beads from the reaction mixture were separated and washed once with DMF before performing the same procedure as in a dry Kaiser test.

### 1.1.5 Capping of Unreacted Resin

For capping of unreacted resin DMF (approx. 5 mL per g resin) was added to the resin, after which subsequently DIPEA (50 eq) and acetic anhydride (50 eq) were added. This was agitated for 5 min, after which the resin was washed with DMF (3× with 10 mL per g resin) and CH₂Cl₂ (3× with 10 mL per g resin).

### 1.1.6 Peptide Cleavage and Deprotection

After each coupling 10 mg resin was agitated with 200 μL 95% TFA, 5% water for 1-3 h. The filtrate was precipitated in cold diethyl ether (0.5 mL) and the solvent was evaporated under nitrogen to give the peptide as a white solid.

### 1.2 Synthesis of Glu-Urea-Lys PSMA Binding Motif

4-alkoxybenzoyl alcohol (Wang) resin (1 eq, 0.8-1.2 mmol/g, 200-400 mesh) was washed 3× with CH₂Cl₂ (10 mL per g resin) and swollen in DMF for 1h. Fmoc-Lys (Alloc)-OH (2 eq) was dissolved in DMF, to which HOBt (4 eq, 1M in DMF) and a solution of DMAP (2 eq) with DIPCDI (2 eq) in DMF were added. This was added to the resin and agitated overnight. The resin was washed 3x with DMF, 3× with CH₂Cl₂ and 3× with diethyl ether (approx. 10 mL per g resin per wash). Of the dry resin the loading was determined as described above. Fmoc-Lys (Alloc)-OH coupling was repeated for a loading lower than 0.5 mmol/g. The resin was swollen in DMF for 10 min, and after Fmoc deprotection as described below, DIPEA (3 eq) was added to the resin. 4-nitrophenyl chloroformate (2 eq) dissolved in DMF was added to the resin and agitated for 1h. A wet and dry Kaiser test were performed as described above and after negative result, CH₂Cl₂ was added to the resin to which DIPEA (4 eq) was added. Ditertbutyl glutamate HCl (3 eq) was dissolved in CH₂Cl₂, added to the resin and agitated for 1 h. The resin was washed 3× with DMF and 3× with CH₂Cl₂ after which a Kaiser test was performed for negative result.

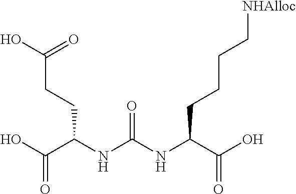

cleavage and deprotection of 10 mg resin-bound Glu-urea-Lys binding motive as a white solid. MS ESI+ (acetonitrile: H₂O 1:1) m/z calcd for [M+H]⁺404.16, found m/z 403.9.

### 1.2a Alternative Synthesis of the PSMA Binding Motif and Linker

i) Wang resin (1 eq., 1.0 mmol/g, 1.00 g) was swollen in 10 mL DMF for 10 minutes. Fmoc-Lys (Alloc)-OH (3 eq., 3 mmol, 1.87 g), 4-dimethylaminopyridine (1 eq., 1 mmol, 122.2 mg), HOBt (3.6 eq., 3.6 mmol, 1M in DMF) and DIPCDI (3.3 eq., 3.3 mmol, 1M in DMF) were added to the resin and mixed on a bench roller for 20 hours. The reagents were removed from the resin by vacuum filtration. The resin was washed with DMF (3×10 mL) and DCM (3×10 mL). The Fmoc-loading was determined to be 0.5 mmol/g. Next, the resin was capped with a solution of pyridine (0.34 mL/g resin) and benzoyl chloride (0.34 mL/g resin) in DCM for 1 hour. ii) The resin was washed with DCM (3×10 mL) and DMF (3×10 mL) and after Fmoc removal DIPEA (0.52 mL, 3 eq., 3 mmol,), 4-nitrophenyl chloroformate (2 eq., 2.0 mmol, 402 mg) in 2 mL DCM were added to the H-Lys (Mtt)-resin (1 eq, 0.5 mmol/g, 2 g) and the resin was agitated for 1 hour. Consecutively a Kaiser test was performed to check for completion. iii) Glutamic acid di-tert-butyl ester hydrochloride (3 eq., 3 mmol, 887.4 mg) and DIPEA (4 eq., 4 mmol, 0.70 mL) in DCM were added to the resin and the mixture was agitated for 1 hour. The resin was washed with DCM (3×10 mL) and DMF (3×10 mL). iv) After Alloc removal, Fmoc-3-(2-naphthyl)-L-alanine (Fmoc-Nal) was coupled using HATU. v) After Fmoc removal either Fmoc-(4-aminomethyl)benzoic acid (Fmoc-Amb) or trans-4-(aminomethyl)cyclohexane-1-carboxylic acid (Fmoc-Amc) was coupled using DIPCDI. vi) After Fmoc removal Fmoc-Lys (Alloc)-OH was coupled using DIPCDI. vii) After Fmoc removal Fmoc-Glu (OtBu)-OH was coupled using DIPCDI. viii) After Fmoc removal nicotinic acid was coupled using DIPCDI. ix) After Alloc removal, Fmoc-Lys (Alloc) was coupled using DIPCDI. x) DIPEA (2 eq.) and DOTA-OSu were added to the resin in NMP and mixed on a bench roller at room temperature respectively for 6-8 hrs. Upon a negative Kaiser test the resin was washed with DMF (3×), DCM (3×), MeOH (3×) and diethyl ether (3×). xi) The peptide was cleaved from the resin with trifluoroacetic acid/H₂O (95:5, v/v) for two hours after which the resin was filtered off and the peptide was precipitated in diethyl ether. After drying in air the crude peptide was lyophilized from water. xii) After Fmoc removal, Fmoc-Gly-OH was coupled. This was repeated twice to couple two more glycines. xiii) After Fmoc removal N-succinimidyl S-acetylthioacetate (SATA, 3 eq.) in DMF was added to the resin. Upon a negative Kaiser test (˜45 minutes) the resin was washed with DMF (3×), DCM (3×), MeOH (3×) and diethyl ether (3×)

### 1.2b Improved Synthesis of Intermediate

li a) The paranitrocloroformamide of Glu (OtBu)-OtBu (1.4 eq., 1.1 mmol, 140 mg) and DIPEA (1.4 eq., 1.1 mmol, 0.2 mL) in 2 mL DCM were added to the H-Lys (Mtt)-resin (1 eq, 0.8 mmol/g, 1.0 g) and the resin was agitated for 1 hour after which the resin was washed with DMF (3×), DCM (3×), MeOH (3×) and diethyl ether (3×)

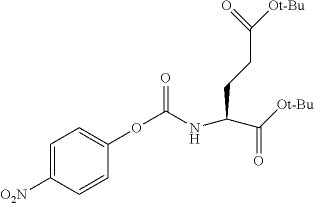 [SMILES: CC(C)(C)OC(=O)CC[C@H](NC(=O)Oc1ccc([N+](=O)[O-])cc1)C(=O)OC(C)(C)C]

li b) In a 25 ml rbf, 4-nitrophenyl carbonochloridate (1.001 g, 0.9787 Eq, 4.966 mmol) was added to a stirring solution of di-tert-butyl L-glutamate hydrochloride (1501 mg, 1 Eq, 5.074 mmol) and DIPEA (1.3 g, 1.8 mL, 2.0 Eq, 10 mmol) in DCM (55 mL) at rt. the solution turned slightly yellow and was quickly cooled to 0 degrees Celsius. After approximately 1.5 hour, the solution was let to warm to room temperature. 3 hours after the start of the reaction, DCM was added to the rm and the organic layer was washed with 1M KHSO4 (aq.) twice, was dried over magnesium sulfate and concentrated in vacuo. The crude product was purified by automated cc (SD4, 30-100% DCM/PE) to obtain di-tert-butyl ((4-nitrophenoxy) carbonyl)-L-glutamate (1483 mg, 3.494 mmol, 68.85%)

### 1.3 Synthesis of PSMA-Binding Peptide Linker

The resin with Glu-urea-Lys PSMA binding motive (1 eq) was swollen in DMF for 10 min, and washed once with CH₂Cl₂. CH₂Cl₂ was added to the resin, which was washed under nitrogen. Phenylsilane (25 eq) and tetrakis(triphenylphosphine) palladium (0)(0.3 eq) were dissolved in CH₂Cl₂ prior to addition to the resin. This was reacted under nitrogen for 30 minutes. The resin was washed with CH₂Cl₂, DMF and sodium diethyldithiocarbamate (0.5% in DMF) till the brown colour had disappeared. A Kaiser test was performed for a positive result. Fmoc-Nal-OH (3 eq) was dissolved in DMF, to which HOBt (3.6 eq, 1M in DMF), DIPEA (6 eq) and HATU (2.9 eq) dissolved in DMF were added. The HATU-containing mixture was shaken for 2 min prior to addition to the resin. The reaction mixture was agitated for 1h, after which the coupling was confirmed by a negative result in a wet and dry Kaiser test. Unreacted resin was capped as described below, after which Fmoc deprotection was performed. After obtaining a positive result, Fmoc-(4-aminomethyl)benzoic acid (3 eq) in CH₂Cl₂ with HOBt (3.6 eq, 1M in DMF) and DIPCDI (3.3 eq, 1M in DMF) was added to the resin and agitated for 1h. A wet and dry Kaiser test were performed for a negative result to confirm the coupling, after which the unreacted resin was capped. Fmoc deprotection was performed before Fmoc-Lys (Mtt)-OH (3 eq) dissolved in DMF, HOBt (3.6 eq, 1 M in DMF) and DIPCDI (3.3 eq in DMF) were added to the resin. This was agitated for 1h, after which coupling was confirmed by a negative wet and dry Kaiser test. In the case of a positive Kaiser test the coupling step was repeated. Any unreacted resin was capped before Fmoc deprotection was performed. Fmoc-Glu (OtBu)-OH (3 eq) was dissolved in DMF to which HOBt (3.6 eq, 1 M in DMF) and DIPCDI (3.3 eq, 1 M in DMF) were added before addition to the resin. This was agitated for 1h, after which a wet and dry Kaiser test were performed.

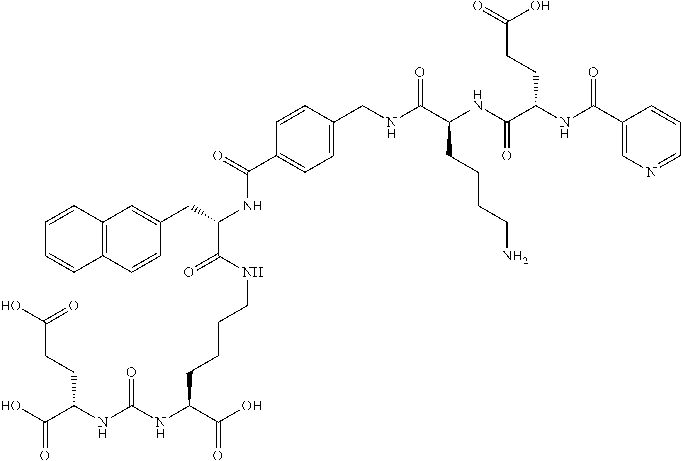 [SMILES: NCCCC[C@H](NC(=O)[C@H](CCC(=O)O)NC(=O)c1cccnc1)C(=O)NCc1ccc(C(=O)N[C@@H](Cc2ccc3ccccc3c2)C(=O)NCCCC[C@H](NC(=O)N[C@@H](CCC(=O)O)C(=O)O)C(=O)O)cc1]

After obtaining negative result, unreacted resin was capped. Fmoc deprotection was performed, after which nicotinic acid (3 eq) dissolved in DMF, HOBt (3.6 eq, 1 M in DMF) and DIPCDI (3.3 eq, 1 M in DMF) were added to the resin. After obtaining negative results in a wet and dry Kaiser test the unreacted resin was capped.

PSMA-binding peptide linker obtained after test cleavage and deprotection of 10 mg resin-bound PSMA-binding peptide linker as a white solid. MS ESI⁺ (acetonitrile: H₂O 1:1) m/z calcd for [M+H]⁺ 1012.43, found m/z 1012.2. HPLC (acetonitrile: H₂O 1:1, 214 nm/254 nm) RT 16.396/16.392 min. LCMS (acetonitrile: H₂O 1:1) ESI⁺ m/z calcd for [M+H]⁺ 1012.43, found RT 13.25 min, m/z 1012.44.

1.4 Synthesis of a Compound Comprising a Linker with Two Functional Groups

The lysine ε-amine of the peptide linker was Mtt deprotected with 1.8% TFA (20 mL per g resin per cycle) until the yellow colour of the liquid phase had disappeared. After washing the resin with DMF (3× with 10 mL per g resin) and CH₂Cl₂ (3× with 10 mL per g resin), either Fmoc-Lys (Boc)-OH (3 eq) (for K5 and K6) or Fmoc-Lys(N₃)—OH (3 eq) (for K8 and K7) was coupled with HOBt (3.6 eq, 1 M in DMF) and DIPCDI (3.3 eq, 1 M in DMF), and unreacted resin was capped. After Fmoc deprotection of the Lysine N-terminus, either a DOTA or MAG₃-Ac chelator was coupled. For coupling of DOTA, to the Lysine N-terminus of the peptide (K5 and K7 on resin, 1 eq) DOTA-NHS-ester (1.2 eq) dissolved in DMF and DIPEA (10 eq) were added and agitated for 2 h. Coupling was confirmed by a wet and dry Kaiser test before continuing. For the coupling of MAG₃-Ac to the Lysine N-terminus of the peptide (K6 and K8 on resin, 1 eq) three cycles of Fmoc-Gly-OH (3 eq) coupling with HOBt (3.6 eq, 1 M in DMF) and DIPCDI (3.3 eq, 1 M in DMF), subsequent capping and Fmoc deprotection were performed, followed by coupling of N-succinimidyl-S-acetylthioacetate (3 eq) in the presence of DIPEA (3 eq). Each coupling was confirmed by a wet and dry Kaiser test. The peptides with either DOTA or MAG₃-Ac chelator were deprotected and cleaved from the resin with cleavage cocktail (10 mL per g resin) for 1 h. For the lysine compounds K5 and K6 the cleavage cocktail was 95% TFA, 5% water, while the azidolysine variants K8 and K7 were cleaved in 95% TFA, 2.5% water, 2.5% TIS. The filtrate was precipitated in cold diethyl ether (6 mL), centrifuged (4000 rpm, 20 min) and the supernatant was discarded. The pellet was dissolved in water/acetonitrile (3 mL, 1:1), purified by reverse-phase HPLC and freeze dried to give the products K5, K6, K7 and K8 as white solids.
K5 obtained from 150 mg resin-bound PSMA-binding peptide linker as a white solid with a yield of 1.6 mg. MS ESI⁺ (acetonitrile: H₂O 1:1) m/z calcd for [M+H]⁺ 1526.71, found m/z 1526. HPLC (acetonitrile: H₂O 1:1, 214 nm/254 nm) RT 15.749/15.750 min.
K6 obtained from 200 mg resin-bound PSMA-binding peptide linker as a white solid with a yield of 2.3 mg. MS ESI⁺ (acetonitrile: H₂O 1:1) m/z calcd for [M+H]⁺ 1427.55, found m/z 1427.3. HPLC (acetonitrile: H₂O 1:1, 214 nm/254 nm) RT 16.597/16.597 min.
K7 obtained from 200 mg resin-bound PSMA-binding peptide linker as a white solid with a yield of 8.8 mg. MS ESI⁺ (acetonitrile: H₂O 1:1) m/z calcd for [M+H]⁺1552.70, found m/z 1552.5. HPLC (acetonitrile: H₂O 1:1, 214 nm/254 nm) RT 17.201/17.203 min. LCMS (acetonitrile: H₂O 1:1) ESI⁺ m/z calcd for [M+H]⁺1552.70, found RT 15.17 min, m/z 1552.52
K8 obtained from 200 mg resin-bound PSMA-binding peptide linker as a white solid with a yield of 1.0 mg. MS ESI⁺ (acetonitrile: H₂O 1:1) m/z calcd for [M+H]⁺1453.58, found m/z 1453.2. HPLC (acetonitrile: H₂O 1:1, 214 nm/254 nm) RT 18.685/18.687 min. LCMS (acetonitrile: H₂O) ESI⁺ m/z calcd for [M+H]⁺1453.58, found RT 17.38 min, m/z 1453.52.

### 1.5 Synthesis of a Compound Comprising a Chelator and a Fluorescent Label

K5 (1.5 eq, 0.77 μmol, 1.18 mg) was dissolved in phosphate buffer solution pH 8 (200 μL). IRDye800CW-NHS ester (1 eq, 0.52 μmol, 172 μL, 3.03 mM in dry DMF) was added and the solution was stirred for 5 h. The mixture was purified by preparative HPLC as described, and freeze dried to give the product K11 as green solid.

K11 obtained from 1.18 mg K5 as a green solid with a yield of <1 mg. MALDI-TOF MS (sinapinic acid 1:1) m/z calcd for [M+H]⁺2511.95, found m/z 2512.3. HPLC (H₂O, 214 nm/254 nm) RT 21.460/21.447 min. Dye-comprising compounds could also be analysed using triethylammonium acetate (10 mM, pH 7) and methanol.

K6 (1.5 eq, 0.79 μmol, 1.13 mg) was dissolved in either phosphate buffer solution pH 7 (200 μL) or phosphate buffer solution pH 8 (200 μL). IRDye800CW-NHS ester (1 eq, 0.53 μmol, 176 μL, 3.03 mM in dry DMF) was added and the solution was stirred for 5 h. The mixture was purified by reverse-phase HPLC with TEA/AcOH (pH7)/MeOH as eluents. The product containing fraction from the pH 7 and pH 8 experiment were mixed and freeze dried to give the product K12 as green solid. K12 obtained from 2.3 mg K6 as a green solid with a yield of <1 mg. MALDI-TOF MS (sinapinic acid 1:1) m/z calcd for [M+H]⁺2412.82, found m/z 2413.2. HPLC (H₂O, 214 nm/254 nm) RT 22.622/22.624 min

Either K7 (1.9 eq, 0.76 μmol, 1.18 mg) or K8 (1.7 eq, 0.67 μmol, 0.97 mg) was dissolved in 200 UL MeCN. IRDye800CW-DBCO (1 eq, 0.40 μmol, 0.54 mg) was dissolved in water (200 μL), added to the peptide solution and mixed for 3 h. The mixtures were purified by reverse-phase HPLC with TEA/AcOH (pH7)/MeOH as eluents, and freeze dried to give the products K13 and K14 as green solids.

K13 obtained from 1.2 mg K7 as a green solid with a yield of <1 mg. MALDI-TOF MS (sinapinic acid 1:1) m/z calcd for [M+H]⁺2814.06, found m/z 2815.5. HPLC (H₂O, 214 nm/254 nm) RT 22.954/22.955 min.

K14 obtained from 1.0 mg K8 as a green solid with a yield of <1 mg. MALDI-TOF MS (sinapinic acid 1:1) m/z calcd for [M+H]⁺2714.94, found m/z 2715.3. HPLC (H₂O, 214 nm/254 nm) RT 23.776/23.778 min

K5 (1 eq, 0.786 μmol, 1.20 mg) was dissolved in phosphate buffer solution pH 8. IRDye 700DX-NHS ester (0.64 eq in dry DMF) was added and the solution was stirred for 6 h. The mixture was purified by preparative HPLC applying a gradient of 20-70% methanol in triethylammonium acetate buffer (10 mM, pH 7), and freeze dried to give the product K15 as blue solid.

K15 obtained as a blue solid with a yield of 1 mg. MALDI-TOF MS (α-Cyano-4-hydroxycinnamic acid 1:1) m/z calcd for [M+H]⁺2278.94, found m/z 2279.07. HPLC (10 mM triethylammonium acetate buffer (pH 7)/MeOH, 214 nm/350 nm) RT 23.073 min

### 1.5 Synthesis of a Compound Comprising a Chelator Suitable for Lead Isotopes

K4 (DOTA): The lysine s-amine of the peptide linker (on resin, 1 eq 0.070 mmol, 200 mg) was Mtt deprotected with 1.8% TFA (4 mL per cycle) until the yellow colour of the liquid phase had disappeared. After washing the resin with DMF (3× with 2 mL) and CH₂Cl₂ (3× with 2 mL), DOTA-NHS-ester (1.2 eq, 0.084 mmol, 71.6 mg) dissolved in DMF (1 mL) was coupled to the resin in the presence of DIPEA (10 eq, 0.70 mmol, 0.12 mL) for 1 h. After confirmation of the coupling by a wet and dry Kaiser test, the peptide was deprotected and cleaved from the resin with 2 mL 95% TFA, 5% water for 1 h. The filtrate was precipitated in cold diethyl ether (6 mL), centrifuged (4000 rpm, 20 min) and the supernatant was discarded. The pellet was dissolved in water (2 mL), purified by reverse-phase HPLC and freeze dried to give the product K4 as a white solid.

K4 obtained from 200 mg resin-bound PSMA-binding peptide linker as a white solid with a yield of 10.0 mg. MS ESI⁺ (acetonitrile) m/z calcd for [M+H]⁺1398.61, found m/z 1398.4. HPLC (acetonitrile: H₂O 1:1, 214 nm/254 nm) RT 16.273/16.273 min

K3 (DO3AM): The lysine ε-amine of the peptide linker (on resin, 1 eq, 0.070 mmol, 200 mg) was Mtt deprotected with 1.8% TFA (4 mL per cycle) until the yellow colour of the liquid phase had disappeared. After washing the resin with DMF (3× with 2 mL) and CH₂Cl₂ (3× with 2 mL), DO3AM-acetic acid (1.5 eq, 0.105 mmol, 42.3 mg) dissolved in DMF (1 mL), HOBt (3.6 eq, 0.253 mmol, 1 M in DMF), DIPCDI (3.3 eq, 0.232 mmol, 1 M in DMF) were added to the resin and agitated for 1.5 h. A wet and dry Kaiser test indicated incomplete coupling, so the coupling was repeated: a mixture of HATU, DO3AM-acetic acid (1.5 eq, 0.105 mmol, 42.3 mg), HOBt (3.6 eq, 0.253 mmol, 1 M in DMF) and DIPEA (6 eq, 0.42 mmol, 73 μL) was shaken for 2 min, added to the resin and agitated for 1 h. After conformation of the coupling by a wet and dry Kaiser test, the peptide was deprotected and cleaved from the resin with cleavage cocktail (2 mL, 95% TFA, 5% water) for 1 h. The filtrate was precipitated in cold diethyl ether (6 mL), centrifuged (4000 rpm, 20 min) and the supernatant was discarded. The pellet was dissolved in water (2 mL), purified by reverse-phase HPLC and freeze dried to give the product K3 as a white solid.

K3 obtained from 200 mg resin-bound PSMA-binding peptide linker as a white solid with a yield of 6.5 mg. MS ESI⁺ (acetonitrile) m/z calcd for [M+H]⁺1395.66, found m/z 1395.5. HPLC (acetonitrile: H₂O 1:1, 214 nm/254 nm) RT 15.865/15.865 min

K2 (TCMC): The peptide linker (on resin, 0.106 mmol, 160 mg) was deprotected and cleaved from the resin with 2 mL, 95% TFA, 5% water for 1.5 h. The filtrate was precipitated in cold diethyl ether (6 mL), centrifuged (4000 rpm, 20 min) and the supernatant was discarded. The pellet was dissolved in water (2 mL), purified by reverse-phase HPLC and freeze dried to give the peptide as a white solid. To the purified peptide (1 eq, 6 μmol, 6.0 mg) in DMF (500 μL) 10.5 μL DIPEA (10 eq, 60 μmol) and 3.3 mg TCMC-Bn-SCN (1 eq, 6 μmol) were added and the solution was stirred for 24 h. The mixture was purified by reverse-phase HPLC and freeze dried to give the product K2 as a white solid.

K2 obtained from 6.0 mg PSMA-binding peptide linker as a white solid with a yield of 5.6 mg. MS ESI⁺ (acetonitrile) m/z calcd for [M+H]⁺1560.70, found m/z 1559.3. HPLC (acetonitrile: H₂O 1:1, 214 nm/254 nm) RT 15.865/15.865 min. LCMS (acetonitrile: H₂O) ESI⁺ m/z calcd for [M+H]⁺ 1560.70, found RT 12.81 min, m/z 1560.16

### 1.6 Benzoic Acid Protected Analogues

### General Procedure Synthesis (4-Aminomethyl)Benzoic Acid (Amb) Linker

After capping, the Lys is deprotected by methyltrityl (Mtt) removal. Fmoc-L-2-napthylalanine-OH (3 eq, 0.75 mmol, 328.11 mg), HOBt (3.6 eq, 0.9 mmol), DIPEA (6 eq, 1.5 mmol, 0.26 mL) and HATU (2.9 eq, 0.725 mmol, 275.67 mg) in DMF added to a falcon tube and HATU was preactivated by shaking the tube thoroughly for two minutes. The preactivated mixture was added to the resin and agitated on a rollerbank for 1 hour. A wet and dry Kaiser test were performed. After capping, Fmoc was removed and Fmoc-(4-aminomethyl)benzoic acid (3 eq, 0.75 mmol, 280.05 mg), HOBt (3.6 eq, 0.9 mmol), DIPEA (6 eq, 1.5 mmol, 0.26 mL) and HATU (2.9 eq, 0.725 mmol, 275.67 mg) in DMF were added to a falcon tube and HATU was preactivated by shaking the tube thoroughly for two minutes. The preactivated mixture was added to the resin and agitated on a rollerbank for 1 hour. A wet and dry Kaiser test were performed. Capping and Fmoc removal preceded coupling Fmoc-Lys (Mtt)-OH (3 eq, 0.75 mmol, 468.6 mg) to the resin with HOBt (3.6 eq, 0.9 mmol) and DIPCDI (3.3 eq, 0.825 mmol) in DMF. The mixture was agitated on a rollerbank for 1 hour and consecutively a wet and dry Kaiser test were performed. The resin was capped and Fmoc was removed from Lys (Mtt)-OH. Fmoc-Glu (OtBu)-OH (3 eq, 0.75 mmol, 425.47 mg), HOBt (3.6 eq, 0.9 mmol) and DIPCDI (3.3 eq, 0.825 mmol) in DMF were added to the resin and the mixture was agitated on a rollerbank for 1 hour. A wet and dry Kaiser test were performed and after capping, Fmoc was removed. Benzoic anhydride (3 eq, 0.75 mmol, 169.67 mg) and DIPEA (3 eq, 0.75 mmol, 0.13 mL) in DMF were added to the resin and the mixture was agitated on a rollerbank for 1 hour. A wet and dry Kaiser test were performed to check if the capping was completed.

### General Procedure Synthesis Tranexamic Acid Linker

Procedure identical to General procedure synthesis 4-(aminomethyl)benzoic acid linker, except that Fmoc-tranexamic acid (3 eq, 0.75 mmol, 284.6 mg) was coupled to napthylalanine, instead of Fmoc-4-(aminomethyl)benzoic acid.

### General Procedure Methyltrityl (Mtt) Removal

1.8% TFA in DCM (10 mL) was added to the resin and agitated on a rollerbank for 5 minutes. The liquid phase is removed by vacuum filtration. These steps were repeated until the solution did not become yellow anymore, but remained colourless after agitation multiple times. A dry Kaiser test was performed to check if deprotection is completed (positive test). Completion of deprotection was also checked by measuring the liquid phase with UV-VIS spectroscopy, using 1.8% TFA in DCM as blanco at wavelength 460 nm. No Mtt protection groups are in the liquid phase if spectrophotometer shows zero absorbance.

### General Procedure Unimodal Ligand (DOTA Chelator)

Mtt was Removed from the Lys Side Chain. DOTA was Coupled to Free NH₂ End.

General procedure precursor bimodal ligand (Fmoc-Lys (Boc)-OH and DOTA chelator) Mtt was removed from the Lys side chain. Fmoc-Lys (Boc)-OH (3 eq), HOBt (3.6 eq) and DIPCDI (3.3 eq) in DMF were added to the resin and agitated on a rollerbank for 1 hour. Consecutively a wet and dry Kaiser test were performed to check if the Fmoc-Lys (Boc)-OH had coupled. Free ends were capped with acetic anhydride (50 eq) and DIPEA (50 eq) in 2 mL DMF for 20 minutes. After the Fmoc group was removed from Lys (Boc), DOTA was coupled to free NH₂ end.

### General Procedure DOTA Coupling to Free NH₂ End, Cleavage and Purification

DOTA-NHS ester (1.2 eq) and DIPEA (10 eq) in DMF are added to resin with binding motif and linker (1 eq) and agitated on a rollerbank for 1 hour. Consecutively a wet and dry Kaiser test were performed to check if the DOTA had coupled. The peptide was cleaved from the resin by adding 5% H₂O in TFA (0.01 L/g resin) and agitating on the rollerbank for 2 hours. The peptide was precipitated in diethyl ether (7 mL) and after centrifugation the pellet was purified with HPLC-prep with 0.1% TFA in Acetonitrile/0.1% TFA in water. The product was freeze dried and obtained as a white solid.

### General Procedure IRDye700DX Coupling (Bimodal)

Precursor (1 eq, 0.00131 mmol, 2.0 mg) was dissolved in potassium phosphate buffer pH 8 (0.2 mL) and IRDye700DX-NHS ester in DMF (0.5 eq, 0.00065 mmol) was added. The reaction mixture was stirred at 20° C. for 6 hours in absence of light.

Conjugation with IRDye800CW

Peptide was dissolved in phosphate buffer (0.25 M, pH 8) after which the dye OSu ester (0.5-0.6 eq. in dry DMF) was added and shaken at rt for 4-6 hrs. The product was purified directly by preparative HPLC.

### Analytical/Preparative HPLC

Compounds were analyzed on a Shimadzu LC-20A Prominence system with a dual UV-Vis detector (Shimadzu, 's Hertogenbosch, The Netherlands) equipped with a C18 Gemini-NX column, 150×3 mm, particle size 3 μm (Phenomenex, Utrecht, The Netherlands) Solvent A was 0.1% trifluoroacetic acid (TFA) in H₂O and solvent B was 0.1% TFA in acetonitrile (MeCN). A gradient of 5-100% acetonitrile (30 min.) was applied. All compounds were purified on a Shimadzu dual-pump LC-20A Prominence system (Shimadzu, 's Hertogenbosch, The Netherlands) equipped with a C18 Gemini-NX column, 150×10 mm, particle size 10 μm (Phenomenex, Utrecht, The Netherlands), applying a gradient of 20-80% methanol in triethylammonium acetate buffer (10 mM, pH 7) for all IRDye containing compounds or a gradient of 5-100% acetonitrile in water (0.1% TFA) for all others.

### General Procedure Indium-111 Labelling

Ligands (PSMA-139, 149, 152, 153, PSMA-617, KWF064) were dissolved in metal-free water in stock solutions. Indium-111 (¹¹¹InCl₃)(0.001 MBq/mL) was added to each ligand (0.001 mg) in MES buffer pH 5.5 (0.5M, 0.005 mL) and incubated at 95° C. for 30 minutes. The labeling efficiency (%) of the ¹¹¹In labelling was quantified with ITLC and phosphorimaging.

### Further Radiolabeling Methods

Indium-111: Ligands (1-20 μg,) were radiolabeled under metal-free conditions with ¹¹¹InCl₃ (Curium) in 0.5 M 2-(N-morpholino) ethanesulfonic acid (MES) buffer (pH 5.5, twice volume of ¹¹¹InCl₃). Labeling was performed at 90° C. for 30 minutes. Ethylenediamine-tetraacetic acid (EDTA, 50 mM) was added to a final concentration of 5 mM after the incubation. Specific activity after labeling ranged from 1-10 MBq/μg. Ligands were purified by a Sep-Pak C18 light cartridge (Waters) and eluted from the cartridge with 50% ethanol in water.

Technetium-99m: Ligands (1-20 μg,) were radiolabeled in 45 μl ammonium acetate (NH₄Ac, 0.25M, pH 8) and 15 μl freshly prepared disodium tartate buffer (50 mg/ml in 0.25M NH₄Ac), under metal-free conditions. Ascorbic acid buffer was prepared just before labeling (3 mg/ml in 10 mM HCl). Next, 5 μl of freshly prepared stannous chloride dihydrate (SnCl₂) buffer (4 μg/ml in ascorbic acid buffer) was added simultaneously with ^{99m}TCO⁻₄ in saline, followed by incubation for 30 min at 90° C. Specific activity after labeling ranged from 1-30 MBq/μg. Ligands were purified by a Sep-Pak C18 light cartridge (Waters) and eluted from the cartridge with 50% ethanol in water.

ITLC/HPLC: Radiochemical yield (RCY) was determined by instant thin-layer chromatography (ITLC) using silica gel-coated paper (Agilent Technologies) and 0.1 M ammonium acetate containing 0.1 M EDTA pH 5.5 (¹¹¹In) or 0.1 M Sodium Citrate pH 6.0 (^{99m}Tc), as the mobile phase. In addition, RCY was measured using reverse-phase high performance liquid chromatography (RP-HPLC) on an Agilent 1200 system (Agilent Technologies) with an in-line radiodetector (Elysia-Raytest). A C18 column (5 μm, 4.6×250 mm; HiChrom) was used at a flow rate of 1 ml/min. We used the following buffer system: buffer A, triethylammonium acetate (TEAA, 10 mM, pH 7); buffer B, 100% methanol; and a gradient of 97% to 0% buffer A (35 min).

Pb-212: compounds 17a, 17b, and 17c were radiolabeled with 212Pb (NRG, Petten) by adding 2.5 M HEPES (4-(2-hydroxyethyl)-1-piperazineethanesulfonic acid, Sigma Aldrich) to 5 kBq ²¹²Pb in 0.1 M HCl and 5 μg ligand. 50 mM EDTA and Tween-80 were added to a final concentration of 5 mM and 0.1%, respectively, after incubation for 35 minutes at 37° C.

ITLC/HPLC: Radiochemical yield (RCY) was determined by instant thin-layer chromatography (ITLC) using silica gel-coated paper (Agilent Technologies) and 0.1 M ammonium acetate containing 0.1 M EDTA pH 5.5, as the mobile phase. In addition, RCY was measured using reverse-phase high performance liquid chromatography (RP-HPLC) on an Agilent 1200 system (Agilent Technologies) with an in-line radiodetector (Elysia-Raytest). A C18 column (5 μm, 4.6×250 mm; HiChrom) was used at a flow rate of 1 ml/min. We used the following buffer system: buffer A, triethylammonium acetate (TEAA, 10 mM, pH 7); buffer B, 100% methanol; and a gradient of 97% to 0% buffer A (35 min).

### Synthesis of Compounds

We synthesized the six PSMA ligands K9, K10, K15, K16, K17, and K18 using solid phase peptide synthesis techniques, with Wang resin as the insoluble porous support. The urea based PSMA binding motif was formed and after the Mtt protecting group was removed from lysine, the hydrophobic linker was synthesized containing either (4-aminomethyl)benzoic acid or tranexamic acid. After capping with benzoic anhydride, the Mtt protecting group of the lysine side chain was removed to couple either a DOTA chelator (unimodal) or Fmoc-Lys (Boc)-OH and a DOTA chelator (bimodal). The peptides were cleaved from the resin and purified with HPLC-prep. The products K9, K10, K16, and K17 were obtained as a white solid.

K10 and K17 are useful precursors for respectively K15 and K18. IRDye700DX was coupled to the free NH₂ ends of the pure precursor and the product was separated from free IRDye700DX and NHS ester with HPLC-prep. After freeze drying the bimodal product was obtained as a blue/green sticky compound. The new ligands were characterized by LCMS, AccuTOF MS and/or MALDI-TOF MS and the purity was checked with analytical HPLC.

K9 obtained from 250 mg resin binding motif-linker as a white solid with a yield of 18.6 mg. LCMS (acetonitrile: H₂O 1:1) ESI⁺ m/z calcd for [M+H]⁺ 1397.63 found RT 15.74 min, m/z 1397.64 HPLC (acetonitrile: H₂O 1:1, 214 nm/254 nm) RT 18.094/18.091 min AccuTOF HRMS (H₂O) ESI+ m/z calcd for [M+H]⁺ 1397.63 found 1397.62651, mass difference 0.24 ppm m/z calcd for [M+Na]⁺ 1419.60977 found m/z 1419.60846, mass difference 0.92 ppm MALDI-TOF MS (alpha-cyano 1:1) m/z calcd for [M+H]⁺ 1397.63* found 1397.634

K10 obtained from binding motif-linker 250 mg resin as a white solid with a yield of 22.8 mg. LCMS (acetonitrile: H₂O 1:1) ESI⁺ m/z calcd for [M+H]⁺ 1525.72 found RT 14.22 min, m/z 1526.28 HPLC (acetonitrile: H₂O 1:1, 214 nm/254 nm) RT 15.571/15.566 min AccuTOF HRMS (H₂O) ESI+ m/z calcd for [M+H]⁺ 1525.72 found m/z 1525.72148, mass difference 2.25 ppm m/z calcd for [M+Na]⁺ 1547.704 found m/z 1547.703.

K15 obtained from K10 as a blue compound. HPLC (phosphate buffer) 350 nm/215 nm) RT 23.138/23.140 min MALDI-TOF MS (alpha-cyano 1:1) m/z calcd for [M+H]⁺ 3363.07 found m/z 2280.07.

K16 obtained from 150 mg binding motif-linker on resin as a white solid with a yield of 13.7 mg. LCMS (acetonitrile: H₂O 1:1) ESI⁺ m/z calcd for [M+H]⁺ 1403.68 found RT 15.80 min, m/z 1403.80 HPLC (acetonitrile: H₂O 1:1, 214 nm/254 nm)—AccuTOF HRMS (H₂O) ESI+ m/z calcd for [M+Na]⁺ 1425.65 found m/z 1425.65541.

K17 obtained from 400 mg binding motif-linker resin as a white solid with a yield of 40.2 mg. LCMS (acetonitrile: H₂O 1:1) ESI⁺ m/z calcd for [M+H]⁺ 1531.77 found RT 14.47 min, m/z 1531.68 HPLC (acetonitrile: H₂O 1:1, 214 nm/254 nm)—AccuTOF HRMS (H₂O) ESI+ m/z calcd for [M+H]⁺ 1531.77 found m/z 1531.76843, mass difference 2.38 ppm m/z calcd for [M+Na]⁺ 1553.75360 found m/z 1553.75038, mass difference 0.34 ppm.

K18 obtained from K17 as a green/blue sticky compound. HPLC (phosphate buffer) 350 nm/215 nm) RT 23.138/23.140 min MALDI-TOF MS (alpha-cyano 1:1) m/z calcd for [M+H]⁺ 3369.12 found m/z 2286.27. K18 is shown below.

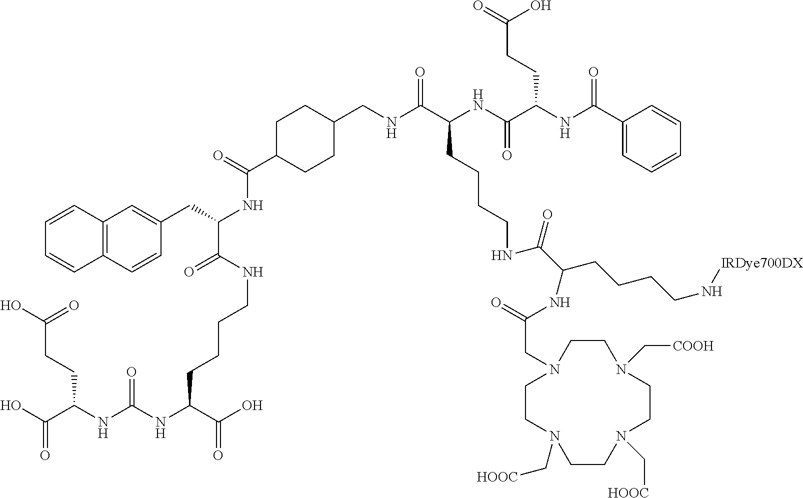

### Synthesis of Additional Compounds

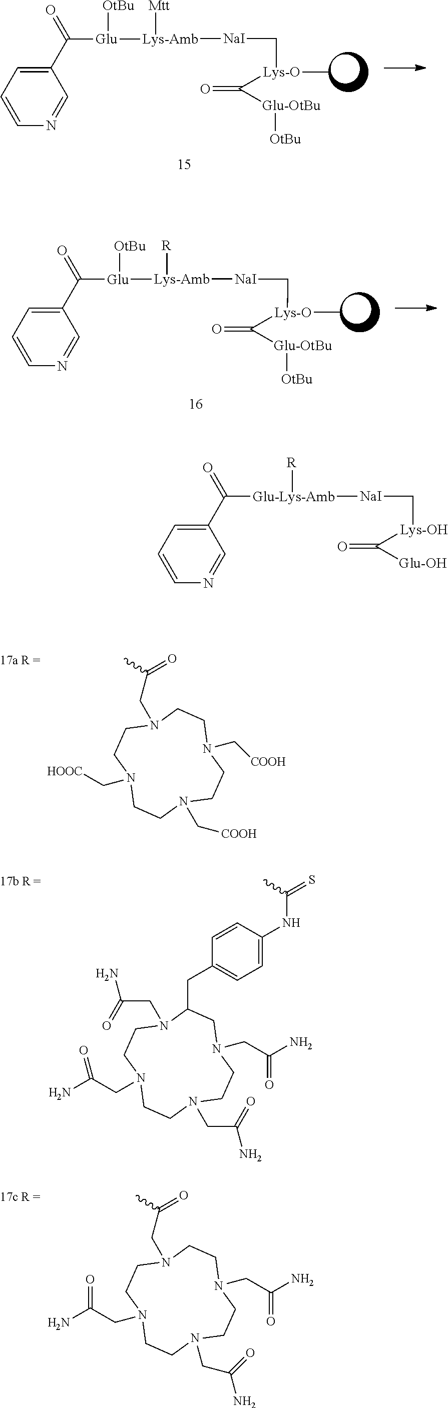

15 was prepared as described above, but instead of Fmoc-Lys (Alloc), Fmoc-Lys (Mtt) was employed in the backbone of the peptide. 17a: The lysine s-amine of the peptide linker (on resin, 1 eq 0.070 mmol, 200 mg) was Mtt deprotected with 1.8% TFA (4 mL per cycle) until the yellow colour of the liquid phase had disappeared. After washing the resin with DMF (3× with 2 mL) and DCM (3x with 2 mL), DOTA-NHS-ester (1.2 eq, 0.084 mmol, 71.6 mg) dissolved in DMF (1 mL) was coupled to the resin in the presence of DIPEA (10 eq, 0.70 mmol, 0.12 mL) for 1 h. After confirmation of the coupling by a wet and dry Kaiser test, the peptide was deprotected and cleaved from the resin with 2 mL 95% TFA, 5% water for 1 h. The filtrate was precipitated in cold diethyl ether (6 mL), centrifuged (4000 rpm, 20 min) and the supernatant was discarded. The pellet was dissolved in water (2 mL), purified by reverse-phase HPLC and freeze dried to give the product as a white solid. 17b: The peptide linker (on resin, 0.106 mmol, 160 mg) was deprotected and cleaved from the resin with 2 mL, 95% TFA, 5% water for 1.5 h. The filtrate was precipitated in cold diethyl ether (6 mL), centrifuged (4000 rpm, 20 min) and the supernatant was discarded. The pellet was dissolved in water (2 mL), purified by reverse-phase HPLC and freeze dried to give the peptide as a white solid. To the purified peptide (1 eq, 6 μmol, 6.0 mg) in DMF (500 μL) 10.5 μL DIPEA (10 eq, 60 μmol) and 3.3 mg TCMC-Bn-SCN (1 eq, 6 μmol) were added and the solution was stirred for 24 h. The mixture was purified by reverse-phase HPLC and freeze dried to give the product as a white solid. 17c: The lysine-amine of the peptide linker (on resin, 1 eq, 0.070 mmol, 200 mg) was Mtt deprotected with 1.8% TFA (4 mL per cycle) until the yellow colour of the liquid phase had disappeared. After washing the resin with DMF (3× with 2 mL) and DCM (3× with 2 mL), DO3AM-acetic acid (1.5 eq, 0.105 mmol, 42.3 mg) dissolved in DMF (1 mL), HOBt (3.6 eq, 0.253 mmol, 1 M in DMF), DIPCDI (3.3 eq, 0.232 mmol, 1 M in DMF) were added to the resin and agitated for 1.5 h. A wet and dry Kaiser test indicated incomplete coupling, so the coupling was repeated: a mixture of HATU, DO3AM-acetic acid (1.5 eq, 0.105 mmol, 42.3 mg), HOBt (3.6 eq, 0.253 mmol, 1 M in DMF) and DIPEA (6 eq, 0.42 mmol, 73 μL) was shaken for 2 min, added to the resin and agitated for 1 h. After conformation of the coupling by a wet and dry Kaiser test, the peptide was deprotected and cleaved from the resin with cleavage cocktail (2 mL, 95% TFA, 5% water) for 1 h. The filtrate was precipitated in cold diethyl ether (6 mL), centrifuged (4000 rpm, 20 min) and the supernatant was discarded. The pellet was dissolved in water (2 mL), purified by reverse-phase HPLC and freeze dried to give the product as a white solid.

Peptides 19a-c: after completion of the backbone, deprotection of the s-amine of the Lys (Alloc) followed and subsequently another Lys (Alloc) was coupled. After removal of this Alloc group, coupling of Ibuprofen (peptide 19b) or 4-pTolylbutyric acid (peptide 19c) was achieved using DIPCDI/HOBt peptide chemistry. In case of peptide 19a, Fmoc-Lys(Ac)-OH was coupled after the ε-amine of the Lys (Alloc) was deprotected. Finally, DO3AM was coupled after which cleavage from the resin yielded peptides 19a-c after HPLC purification.

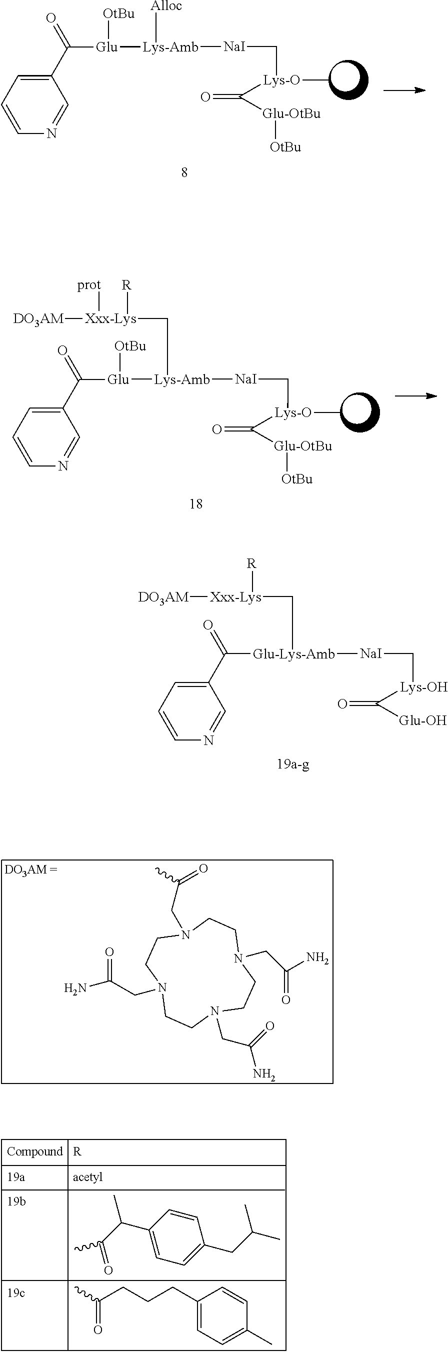

### Example 2—Characterisation of Compounds According to the Invention

### 2.1 Method

PSMA-expressing LS174T-PSMA cells were plated at 1.25×10⁶ cells per well in three six-well plates in culture medium (RPMI with 1% glutamine, 10% fetal calf serum, 0.3 mg/mL) (3 mL/well). After two days incubation at 37° C. and 5% CO₂, the cells were washed twice with binding buffer (RPMI with 0.5% bovine serum albumin). PSMA-ligands K4, K3, and K2 (1 g/L, 1 μL) each were mixed with metal-free MES buffer (6 μL, 0.5 M, pH 5.5) and ¹¹¹InCl₃ (3 μL, 1 MBq/μL in HCl) and incubated at 95° C. for 45 minutes after which EDTA (1 μL, 50 mM) was added. Labelling efficiency of the PSMA-ligands was determined by means of ITLC in ammonium acetate buffer. PSMA-ligands with more than 10% free ¹¹¹In were purified using a Seppak cartridge. Three wells of each six-well plate were incubated with PMPA (5 μL/well, 10 μg/uL in metal-free water) and labelled PSMA-ligand (50,000 cpm in 100 μL buffer/well), while the other three were incubated with labelled PSMA-ligand (50,000 cpm in 100 μL MeCN/well) only. Incubation was done for 2 h at 37° C. and 5% CO₂. Cells were washed twice with ice-cold PBS (2 mL/well per wash) and lysed with NaOH (2 mL/well, 0.1 M), after which the radioactivity was measured in a γ-counter.

Alternative internalization assay: binding and internalization characteristics of all ligands were compared using LS174T-PSMA and wildtype LS174T cells, cultured to confluency in 6-wells plates. Cells were incubated with 50,000 counts per minute (cpm) ¹¹¹In- or ^{99m}Tc-labeled PSMA ligand (0.1-0.25 pmol/well) in 1 ml binding buffer (RPMI/0.5% BSA) for 2 h at 37° C. Nonspecific binding was determined by co-incubation with 2-(phosphonomethyl) pentane-1,5-dioic acid (2-PMPA, 21.57 UM). Cells were washed with PBS and incubated with acid buffer (0.1 M acetic acid, 154 mM NaCl, pH 2.6) for 10 minutes at 0° C. to retrieve the membrane-bound fraction. After this, the membrane-bound fraction was collected, cells were washed, lysed with 1.5 ml 0.1 M NaOH and cell lysis (intracellular activity) was collected. Membrane-bound activity and intracellular activity fractions were measured in a gamma-counter (2480 WIZARD² Automatic Gamma Counter, PerkinElmer) (6,42).

IC₅₀: The 50% inhibitory concentration (IC₅₀) of the ligands was determined using LS174T-PSMA cells in a competitive binding assay. The LS174T-PSMA cells were cultured to confluency in 6-wells plates, followed by incubation on ice for 2 h in 1 mL of binding buffer (RPMI/0.5% BSA) with 50,000 cpm of ¹¹¹In-labeled PSMA-617 and a series of increasing concentrations (0.01-300 nM) of unlabeled PSMA ligands. After incubation, cells were washed with 2 ml PBS and lysed with 1.5 ml 0.1 M NaOH. Cell lysis was collected from the plate and the cell-associated activity was measured in a gamma-counter and IC₅₀ values were calculated using GraphPad Prism software version 5.03. Lipophilicity: LogD values of all radiolabeled ligands were determined by adding 300,000 cpm (0.6-1.5 pmol) to a mixture of 3 ml PBS (pH 7.4) and 3 ml n-octanol. Tubes were vortexed vigorously for 1 min and centrifuged for 5 min at 201×g. The concentration of radioactivity in a defined volume of each layer was measured in a well-type gamma-counter.

Binding assay using albumin-binding moieties and related compounds: binding characteristics of 17a, 17b, and 17c, and of 19a, 19b, and 19c were compared using LS174T-PSMA cells, cultured to confluency in 6-wells plates. Cells were incubated with 15,000 cpm ²¹²Pb-labeled PSMA ligand in 2 ml binding buffer (RPMI/0.5% BSA) for 2 h at 37° C. Nonspecific binding was determined by co-incubation with 2-PMPA (50 μg). Cells were washed with PBS, lysed with 1.5 ml 0.1 M NaOH, and cell lysis (intracellular activity) was collected. Membrane-bound activity and intracellular activity fractions were measured in a gamma-counter (2480 WIZARD2 Automatic Gamma Counter, PerkinElmer).

Internalization assay of the above: internalization characteristics of modified PSMA ligands were compared using LS174T-PSMA cells and ¹¹¹In-labeled ligands. Ligands were labeled as described herein. Cell were cultured to confluency in 6-wells plates. Next, cells were incubated with 50,000 counts per minute (cpm) ¹¹¹In-labeled PSMA ligand in 2 ml binding buffer (RPMI with 0.5% BSA) for 2 h at 37° C. Binding and internalization kinetics for the PSMA ligands with an albumin-binding motif (DO3AM-PSMA-Ibu (19b) and DO3AM-PSMA-p-tolyl (19c)) were compared with and without the addition of BSA to determine any interference of the BSA during the assay. Nonspecific binding was determined by co-incubation with 2-PMPA (50 μg). Cells were washed with PBS and incubated with acid buffer (0.1 M acetic acid, 154 mM NaCl, pH 2.6) for 10 minutes at 0° C. to retrieve the membrane-bound fraction. After this, the membrane-bound fraction was collected, cells were washed, lysed with 1.5 ml 0.1 M NaOH and cell lysis (intracellular activity) was collected. Membrane-bound activity and intracellular activity fractions were measured in a gamma-counter (2480 WIZARD2 Automatic Gamma Counter, PerkinElmer).

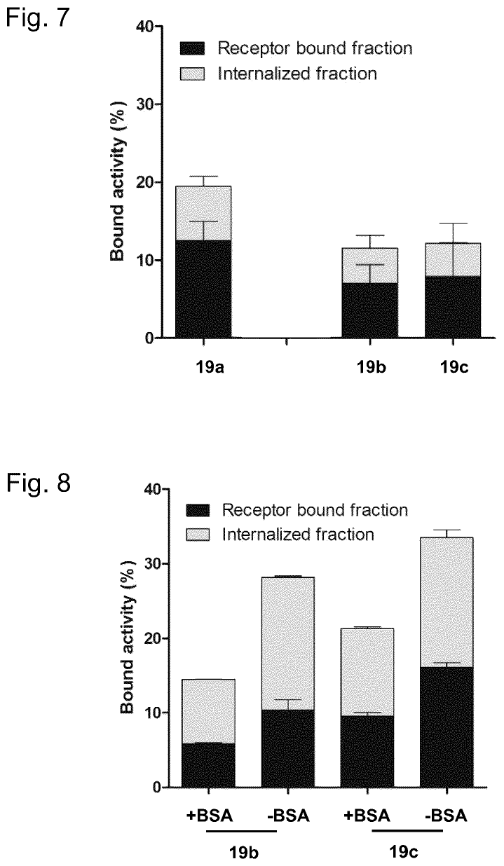

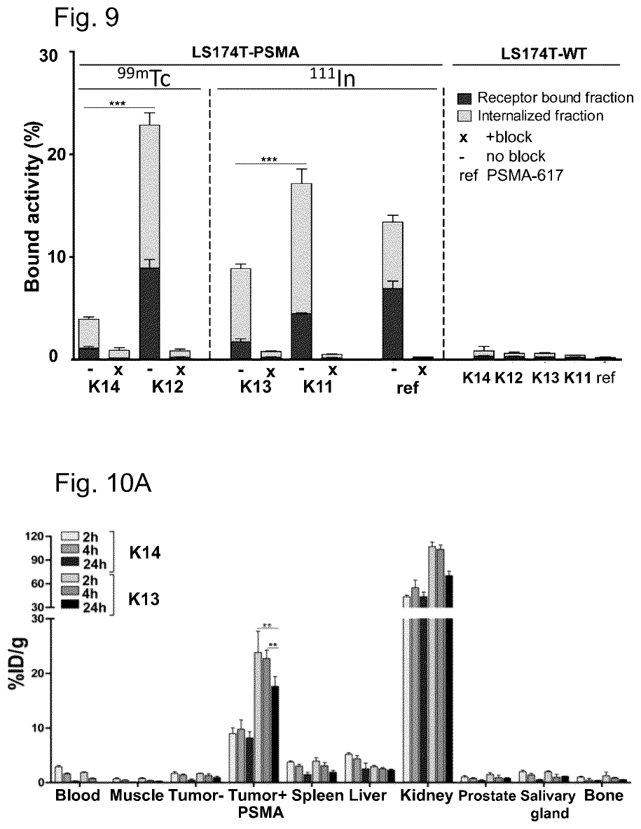

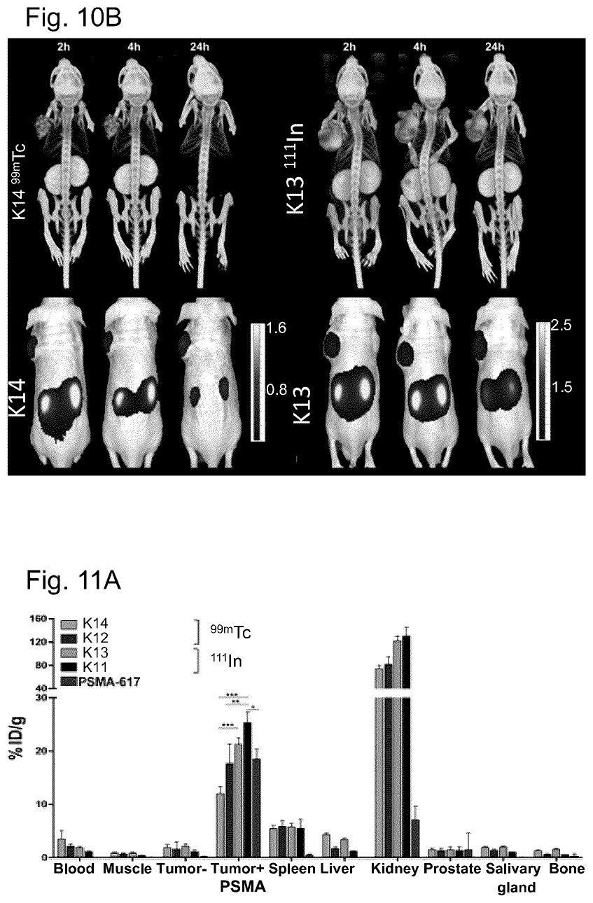

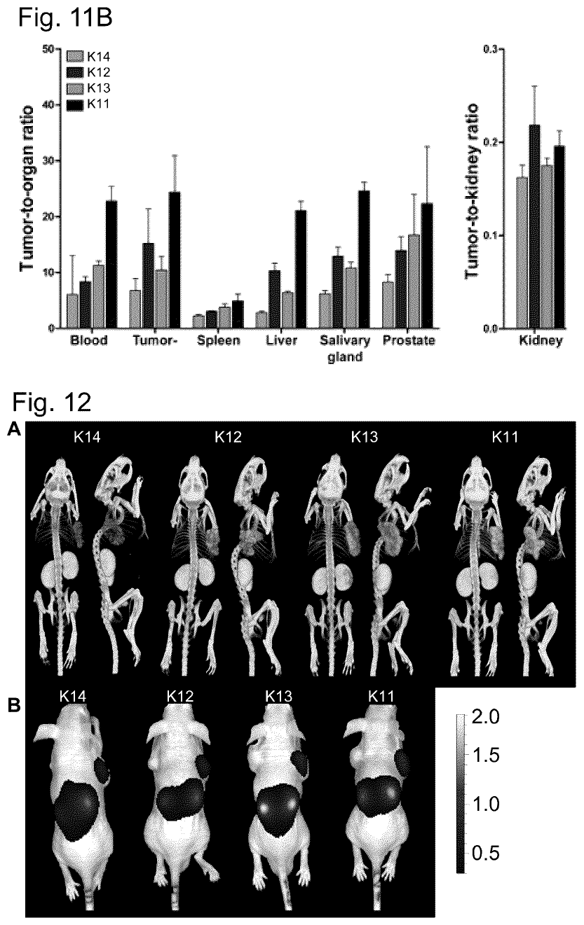

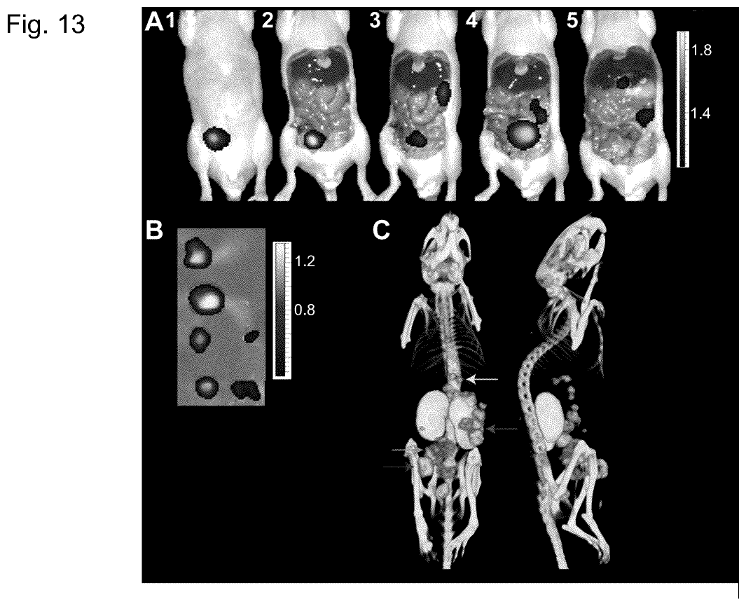

Results for the above two paragraphs are shown in FIG. 6, FIG. 7, and FIG. 8.

Subcutaneous tumor model: Animal experiments were performed in 8-10 weeks old male BALB/c nude mice (Janvier). Animals were housed under non-sterile conditions in individually ventilated cages (Blue line IVC, 4-5 mice per cage) with cage enrichment present and free access to water and chlorophyll-free animal chow (Sniff GmbH). Mice were subcutaneously inoculated with 3.0×10⁶ LS174T-PSMA cells in the right flank and 1.5×10⁶ LS174T wildtype cells in the left flank, diluted in 200 μL of complete RPMI 1640 medium. When xenografts were approximately 0.1 cm³ (10-14 days after tumor inoculation), tracers were injected intravenously in the tail vain. The biotechnicians performing the s.c. and i.v. injections were blinded for the experimental groups and tumor-bearing mice were block-randomized into groups based on tumor size. All experiments were conducted in accordance to the guidelines of the Revised Dutch Act on Animal Experimentation and approved by the institutional Animal Welfare Committee of the Radboud university medical center. Biodistribution, fluorescence imaging and μSPECT/CT imaging: Mice were injected intravenously with 10 MBq ¹¹¹In-labeled PSMA-N048, -N050 or PSMA-617 as control (0.3 nmol, molar activity 33.3 MBq/nmol) or 15 MBq ^{99m}Tc-labeled PSMA-N049 or -N050 (0.3 nmol, molar activity 50 MBq/nmol) in PBS/0.5% BSA. Two hours post injection (p.i.), mice were euthanized by CO₂/O₂ asphyxation and images were acquired with the IVIS fluorescence imaging system (Xenogen VivoVision IVIS Lumina II, PerkinElmer), using an acquisition time of 30 s. Subsequently, μSPECT/CT images were acquired (U-SPECT II, MILabs) with a 1.0 mm diameter pinhole mouse collimator tube. Mice were scanned for 30 min followed by a CT scan (spatial resolution 160 μm, 65 kV, 615 μA) for anatomical reference. μSPECT/CT scans were reconstructed with MILabs reconstruction software, using an ordered-subset expectation maximization algorithm, energy windows 154-188 keV and 220-270 keV for ¹¹¹In, and 126-154 keV for ^{99m}Tc, 1 iteration, 16 subsets, voxel size of 0.4 mm. μSPECT/CT scans were analyzed and maximum intensity projections (MIPs) were created using the Inveon Research Workplace software version 4.1 (Siemens Preclinical Solutions). NIRF images were analyzed using Living Image software version 4.2 (Caliper Life Sciences). Tumors, blood, and relevant organs and tissues were dissected, weighed, and radioactivity in each sample was quantified using a well-type gamma-counter. The results were expressed as percentage of injected dose per gram of tissue (% ID/g). Pharmacokinetics: To determine the pharmacokinetics of the ligands, nine groups of five mice received an intravenous injection of 0.3 nmol ¹¹¹In-labeled PSMA-N048 or PSMA-N050 (10 MBq/mouse, molar activity 33.3 MBq/nmol) or ^{99m}Tc-labeled PSMA-N049 (5 MBq/mouse, molar activity 16.7 MBq/nmol) in PBS/0.5% BSA. At 2, 4, and 24 h p.i. mice were euthanized followed by dissection. Tissues of interest were dissected, weighed and measured for radioactivity in a gamma-counter as described above. For each ligand, two mice from the 24 h groups underwent repeated μSPECT/CT and NIRF imaging (2, 4 and 24 h p.i.). During imaging, mice were anesthetized with 2.5% isoflurane inhalation anesthesia and kept warm with a heating pad. Images were acquired and analyzed as described above.

Intraperitoneal tumor model: LS174T-PSMA cells (1.0×10⁶) in 200 μl of complete RPMI 1640 medium were injected intraperitoneally and grew for 28 d after inoculation. Six male BALB/c nude mice with intraperitoneally growing LS174T-PSMA tumors were intravenously injected with ¹¹¹In-labeled PSMA-N050 (10 MBq and 0.3 nmol/mouse). Two hours p.i., μSPECT/CT imaging was performed preoperatively (30 min), followed by NIRF imaging of the mice in supine position after surgical removal of skin, abdominal muscle layers, and peritoneum. After in vivo image acquisition, the visualized tumors were resected, followed by NIRF imaging to control whether residual tumor tissue was in situ.

### 2.2 Result for Cell Binding

PSMA-ligands K4, K3, and K2 were radiolabelled with gamma-emitting 111In to monitor the binding and internalization of the ligands with different chelators on PSMA-expressing LS174T-PSMA cells. Labelling efficiencies of K4, K3, and K2 were 100%, 70% and 54%, respectively. Since this indicates that still a relatively large amount of free 111In is present in the ligand solution of K3 and K2, labelled K3 and K2 were purified by solid phase extraction before incubation with cells. The results of the cell binding assay can be seen in FIG. 2, which shows per ligand the amount of γ-emission counts remaining after incubation and washing of the cells, which indicates binding and/or internalization of the radiolabelled ligands. For K4, K3, and K2 this resulted in 27.5±1.0%, 20.9±0.4% and 13.8±0.3% remaining, respectively. Emission could be blocked almost completely when PSMA was blocked with PMPA, a high-affinity PSMA-inhibitor, indicating that nonspecific binding of the three PSMA-ligands with different chelators was very low and binding was specifically to PSMA. The differences between the ligands with different chelators with respect to binding to PSMA could be due to a lower radiochemical purity of K3 and K2 compared to K4, which may have resulted in free ¹¹¹In in the sample leading to an erroneously higher standard that was set to 100%. It can be concludes that all three ligands with different chelators are capable of specifically binding PSMA.

### 2.3 Cell Binding Results Using Bimodal Compounds

2.3.1 General Procedure Count Calculation with Standards

Each labelled ligand (0.01 mL) was dissolved in PBS (0.5 mL) and diluted 1:10:100 in three tubes, resulting in tube 1 1×, tube 2 10× and tube 3 100× dilution. The tubes were measured by a gamma-counter to determine the counts per minute of known ligand concentrations. The calculated amounts of ligand were transferred from tubes 2 into new tubes and filled with binding buffer to total volume 0.5 mL. These solutions are needed for the binding and internalization assay.

### General Procedure Binding and Internalization Assay

Two days before the assay, four six-well plates were plated with 0.3-1.0 million PSMA-expressing LS174T-PSMA cells per well in culture medium (2 mL). On the day of the assay, the cells were washed with PBS. Binding buffer (0.5% BSA in RMPI 1640, 2 mL) and 50,000 counts per minute of radiolabelled PSMA-ligand (0.005 mL) were added from tubes prepared according to calculation with standards. Each ligand was examined in triplicate. The cells were incubated at 37° C. for 2 hours and after this the medium was removed. The cells were washed with ice-cold PBS and cooled to 0° C. After incubating the cells with acid buffer pH 2.6 (0.1M acetic acid, 0.15 M NaCl, 2 mL) for 10 minutes, the membrane-bound ligands were collected in tubes. The cells were washed with ice-cold PBS again and these fractions were collected as control. NaOH was added to the cells (0.1 M, 2 mL) for 10 minutes to lyse the cells and free the internalized ligands. The ¹¹¹In-radiocounts per minute of all fractions and standards were measured by a gamma-counter. From this, the ligand-PSMA binding percentages and internalization fraction were quantified.

### General Procedure Binding Specificity Assay

PMPA was added to half of the wells of K15 and K18 to block aspecific binding of the ligands to the PSMA-expressing cell culture. Rest of assay is performed following the procedure of binding and internalization assay. Counts of the wells treated with PMPA were compared with the wells of K15 and K18 without PMPA, examining whether the ligand-PSMA binding is specific or not.

### Results

PSMA-expressing LS174T-PSMA cells were incubated with the labelled ligands. Table 2.2.1 shows the results of the binding and internalization assay. We would obtain a binding percentage of 100% if all added ligands (50,000 counts per minute) have bound to the PSMA expressing cells. All ligands that are not bound to cells after incubation were removed with PBS. Ligands K9 and K16 bound and internalized with the highest total percentages into the PSMA expressing cells, respectively 18.9% and 15.1%. For these unimodal ligands around 50% of the ligand internalized after binding. Comparison to PSMA-617, which is used for clinical therapy, showed that the new ligands bound 2.6 and 2.1 times better to the PSMA expressing cells than PSMA-617.

TABLE 2.2.1

| Average binding % | K9 | K16 | K15 | K18 | PSMA-617 |
|---|---|---|---|---|---|
| Membrane bound | 9.87 | 7.92 | 1.41 | 0.52 | 3.80 |
| Internalized | 9.06 | 7.18 | 7.97 | 4.80 | 3.39 |
| Total | 18.93 | 15.10 | 9.38 | 5.32 | 7.19 |

Bimodal ligand K15 had a total binding percentage of 9.38% in this first cell assay. 85% of the bound ligand internalized after binding, which can be useful to avoid fast clearance from the body, as described in the introduction section. Compared to PSMA-617, which is used for clinical therapy, K15 showed a 1.3 times higher total binding. Combined with the idea that K15 is bimodal and has a much higher internalization degree, K15 and similar compounds according to the invention are very useful.

On the contrary, bimodal K18, which has tranexamic acid in its linker instead of benzoic acid, showed a total binding percentage of 1.65%.

We also determined the binding specificity of the bimodal K15 in this cell assay. The results are shown in Table 2.2.2. The total membrane bound percentage is 1.41% and the internalized fraction was 7.97%, as was already shown before. The cells that were treated with PMPA and thus allow no aspecific ligand binding had 1.24% membrane bound ligands and 7.57% internalized ligands. This means that in both cases less than 1% was aspecifically bound. Most ligand binding is thus PSMA specific, which is required as we want to target prostate tumour cells and its metastases, not healthy cells.

TABLE 2.2.2

| Average binding speciticity % | K15 |
|---|---|
| Total membrane bound | 1.41 |
| Specifically membrane bound | 1.24 |
| Total internalized | 7.97 |
| Specifically internalized | 7.57 |

### 2.4 Indium Labelling Results

The comprised chelators were loaded with the radionuclide Indium-111. The labelling was tested by instant TLC. Compounds with Indium-111 labelled ligands stay on the baseline during iTLC, while the spots for free Indium run to the top of the plates. By phosphorimaging the labelling percentages were quantified. These labelling percentages, shown in Table 2.4.1, can be useful to be able to calculate how much ligand must be added to the PSMA-expressing cells during binding and internalization assays.

TABLE 2.4.1

| Compound | Indium-111 labelling |
|---|---|
| K9 | 72% |
| K15 | 93% |
| K16 | 85% |
| K18 | 84% |

IC₅₀ values of ligands were determined in competitive binding assays using LS174T-PSMA cells. Lipophilicity of ligands were expressed in logD values. The internalization ratio was determined in LS174T-PSMA cells. Results are shown in table 2.4.2.

TABLE 2.4.2
In vitro characterization of K11, K12, K13, and K14

| | | | Internalization |
|---|---|---|---|
| Name | IC₅₀ (nM) | LogD | Ratio (%) |
| K13 | 184.4 (95% CI: | −3.2 +/− 0.16 | 80.6 +/− 11.6 |
| | 91.0-373.6) | | |
| K14 | 358.7 (95% CI: | −2.4 +/− 0.39 | 71.9 +/− 13.7 |
| | 226.4-568.4) | | |
| K11 | 312.3 (95% CI: | −4.4 +/− 0.01 | 74.1 +/− 20.3 |
| | 180.8-539.3) | | |
| K12 | 474.9 (95% CI: | −3.5 +/− 0.01 | 61.0 +/− 12.8 |
| | 271.6-830.5) | | |
| PSMA-617 | 8.5 (95% CI: | −4.1 +/− 0.01 | 48.4 +/− 12.4 |
| | 5.1-14.4) | | |

### Example 3—In Vivo Characterisation

### 3.1 Material and Methods

### 3.1.1 Cell Culture

Cell lines were purchased from the American Type Culture Collection. LS174T colon carcinoma cells were stably transfected with human PSMA using a plasmid (see Journal of nuclear medicine. 2014; 55 (6): 995-1001) and were cultured in RPMI 1640 medium supplemented with 2 mM glutamine, 10% FCS, and 0.3 mg/ml G418 at 37° C. in a humidified atmosphere with 5% CO₂.

### 3.1.2 Radiolabeling

K15, K19, and K18 were radiolabeled with ¹¹¹InCl₃ (Curium) in 0.5 M 2-(N-morpholino) ethanesulfonic acid (MES) buffer (twice volume of ¹¹¹InCl₃), pH 5.5, for 10-30 min at 45° C. under metal-free conditions (2). Following incubation, 50 mM ethylenediaminetetraacetic acid (EDTA) was added to a final concentration of 5 mM to chelate unincorporated ¹¹¹InCl₃. Labeling efficiency was determined by instant thin-layer chromatography (ITLC) using silica gel-coated paper (Agilent Technologies) and 0.1 M ammonium acetate containing 0.1 M EDTA, pH 5.5, as the mobile phase. Moreover, radiochemical purity was checked using reverse-phase high performance liquid chromatography (RP-HPLC) on an Agilent 1200 system (Agilent Technologies) with an in-line radiodetector. A C18 column (5 μm, 4.6×250 mm; HiChrom) was used at a flow rate of 1 ml/min with the following buffer system: buffer A, triethylammonium acetate (10 mM, pH 7); buffer B, 100% methanol; and a gradient of 97% to 0% buffer A (35 min). Peptides were purified by a Sep-Pak C18 light cartridge (Waters) and eluted from the cartridge with 50% ethanol in water.

### 3.1.3 In Vitro Assays

Binding and internalization characteristics of all ligands were compared using an LS174T cell line transfected with PSMA (LS174T-PSMA) and wildtype LS174T cells. Cells were cultured to confluency in 6-wells plates followed by incubation at 37° C. for 2 h in 1 ml binding buffer (RPMI/0.5% BSA) with 50,000 counts per minute (cpm) of ¹¹¹In-labeled ligand (0.1-0.25 pmol/well). Nonspecific binding was determined by coincubation with 2-(phosphonomethyl) pentane-1,5-dioic acid (2-PMPA, 21.57 μM). PSMA-specific binding was defined as nonspecific binding subtracted from total binding. To retrieve the membrane-bound fraction, cells were washed with PBS twice and incubated with acid buffer (0.1 M acetic acid, 154 mM NaCl, pH 2.6) for 10 minutes at 0° C. After incubation, the membrane bound fraction (in acid buffer) was collected, cells were washed, lysed with 0.1 M NaOH and cell lysis (intercellular activity) was collected. Membrane-bound activity and intercellular activity fractions were measured in a gamma-counter (2480 WIZARD2 Automatic Gamma Counter, PerkinElmer) (see Contrast media & molecular imaging. 2015; 10 (1): 28-36).

### 3.1.4 Animal Tumor Model

Animal experiments were performed in 8-10 weeks old male BALB/c nude mice (Janvier). Animals were housed in individually ventilated cages (Blue line IVC, 4-5 mice per cage) under nonsterile standard conditions with cage enrichment present and free access to chlorophyll-free animal chow (Sniff GmbH) and water. Mice were subcutaneously inoculated with 3.0×10⁶ LS174T-PSMA cells in the left flank and 1.5×10⁶ LS174T cells in the right flank, diluted in 200 UL of complete RPMI 1640 medium. Eleven days after tumor cell inoculation when xenografts were approximately 0.1 cm³, tracers were injected intravenously via the tail vein. The researchers were not blinded for the experimental groups and tumor-bearing mice were block-randomized into groups based on tumor size. All experiments were approved by the institutional Animal Welfare Committee of the Radboud university medical center and were conducted in accordance to the guidelines of the Revised Dutch Act on Animal Experimentation.

### 3.1.5 Dual-Modality μSPECT/CT, NIRF Imaging and Biodistribution

Mice were injected intravenously with 10 MBq ¹¹¹In-labeled PSMA ligands (0.3 nmol, molar activity 33.3 MBq/nmol) in PBS/0.5% BSA. Two hours post injection, mice were euthanized by CO₂/O₂-asphyxation and images were acquired with the IVIS fluorescence imaging system (Xenogen VivoVision IVIS Lumina II, Caliper Life Sciences), using an acquisition time of 10 s. Subsequently, μSPECT/CT images were acquired (U-SPECT II, MILabs) with a 1.0 mm diameter pinhole mouse collimator tube (Journal of nuclear medicine, 2009; 50 (4): 599-605). Mice were scanned for 30 min followed by a CT scan (spatial resolution 160 μm, 65 KV, 615 μA) for anatomical reference. μSPECT/CT scans were reconstructed with MILabs reconstruction software, using an ordered-subset expectation maximization algorithm, energy windows 154-188 keV and 220-270 keV, 3 iterations, 16 subsets, voxel size of 0.4 mm. SPECT/CT scans were analyzed and maximum intensity projections (MIPs) were created using the Inveon Research Workplace software version 4.1 (Siemens Preclinical Solutions). NIRF images were analyzed using Living Image software version 4.2 (Caliper Life Sciences). Tissues of interest were dissected, weighed and measured for radioactivity in a gamma-counter as described above.

### 3.1.6 In Vitro Targeted Photodynamic Therapy

Wildtype LS174T and LS174T-PSMA cells were cultured to confluency in 48-well plates. Cells were incubated for 2 h (5% CO₂, 37° C.) with 30 nM PSMA ligand in binding buffer (RPMI 1640 medium with 0.5% BSA) in triplicate. The triplicates were randomly distributed over the center of the plates considering the variation in light intensity within the NIR LED device (Journal of nuclear medicine,. 2014; 55 (11): 1893-8). As negative control for NIR light irradiation effects, cells that only received PBS without PSMA ligand were included. After washing with PBS, 0.5 ml fresh binding buffer was added to each well. Subsequently, cells were irradiated with a NIR LED that emits light at a wavelength of 670 to 710 nm (5). The typical forward voltage was 2.6 V, creating a power output of 490 mW using 126 individual LED bulbs to ensure homogenous illumination of the area of interest predefined as 5×3 cm. The cells were irradiated at NIR radiant exposures of 100 J/cm² and subsequently incubated for 1 h at 37° C. As control for cellular toxicity of the PSMA ligands, cells incubated with PSMA ligand that were not irradiated with NIR light were also included as an experimental group. Cytotoxic effects of PDT with PSMA ligands were determined with a CellTiter-Glo® assay (Promega Benelux) according to the manufacturer's instructions. Binding buffer was replaced with 100 μl fresh binding buffer and 100 μl CellTiter-Glo® 2.0 Assay. Plates were shaken (2 min) and incubated for 10 min at room temperature. To determine the metabolic activity of the cells, the luminescence was measured in a plate reader (Infinite® 200 PRO, Tecan).

### 3.1.7 Statistical Analysis

Statistical analyses were performed with Graphpad Prism, version 5.03. Results are presented as mean±SD. Differences in in vitro affinity and in vivo tumor and organ uptake were tested for significance using a one-way ANOVA with a Bonferroni's multiple comparison posttest. Differences were considered significant at P<0.05, two-sided.

### 3.2 Results

Table 3.2.1 shows membrane bound and internalized fractions for compound K19.

TABLE 2
Name, structure, membrane bound and internalized
fractions of the multimodal PSMA ligands.

| | | | FRACTION | |
|---|---|---|---|---|
| NAME | Chelator | IRDye700DX | Membrane bound | Internalized |
| K19 | DOTA | Yes | 1.9 ± 0.1 | 4.5 ± 0.3 |

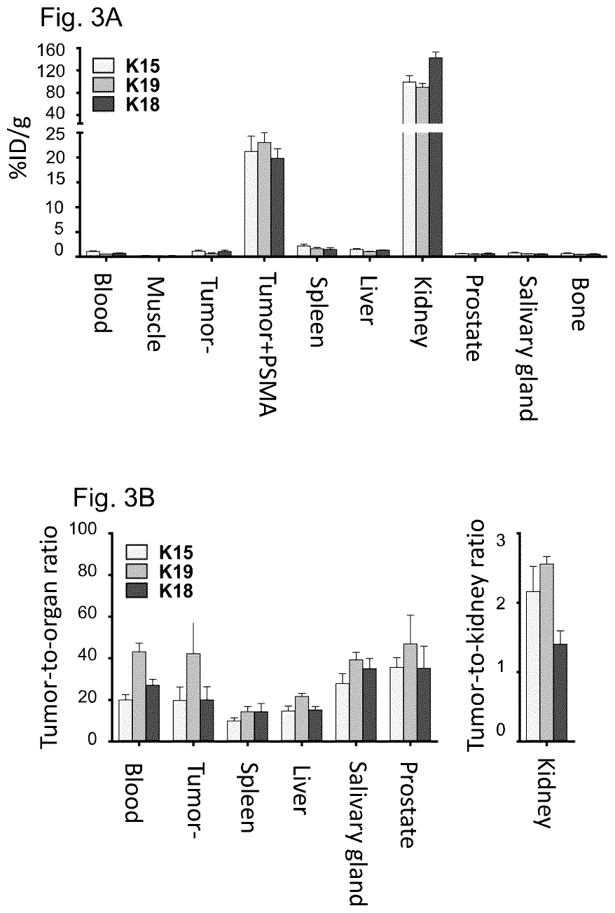

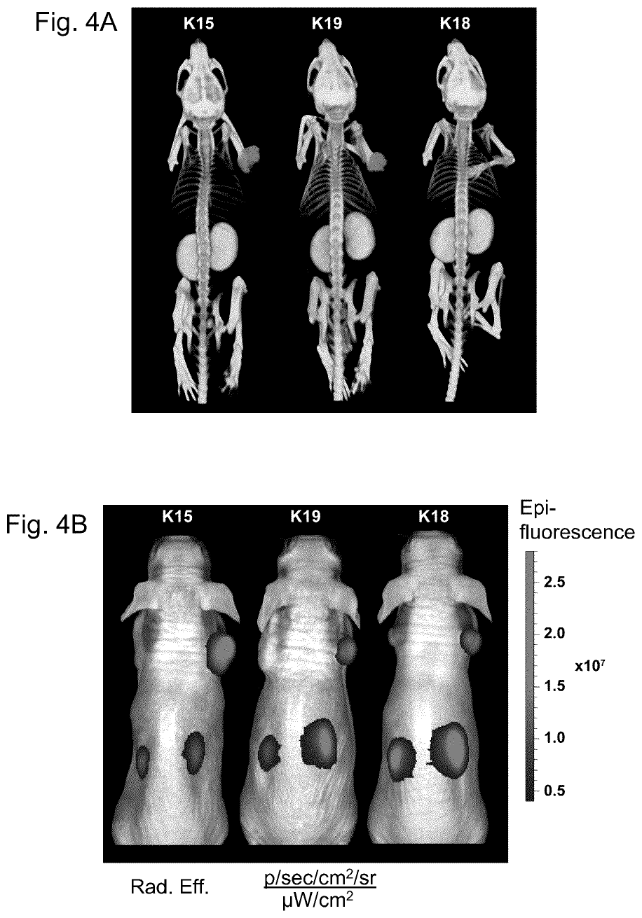

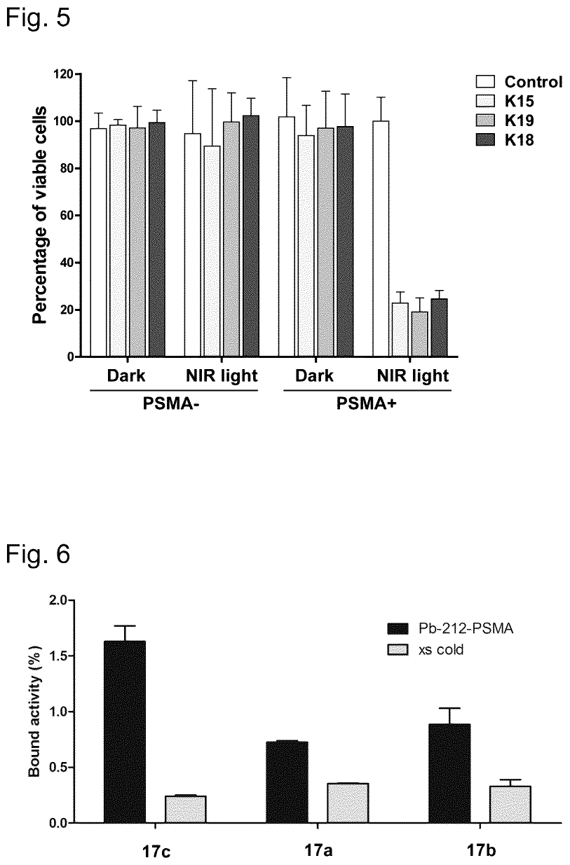

FIG. 3 shows biodistribution and resulting tumor-to-organ ratios for K15, K18, and K19. The Reference compound is an analogue of K15 wherein the glutamic acid moiety is absent from the PSMA-binding motif. μSPECT/CT and fluorescence images of these compounds are shown in FIG. 4, and FIG. 5 shows photodynamic therapy potential of these three compounds.

1. A compound of general formula (1) or a salt thereof: 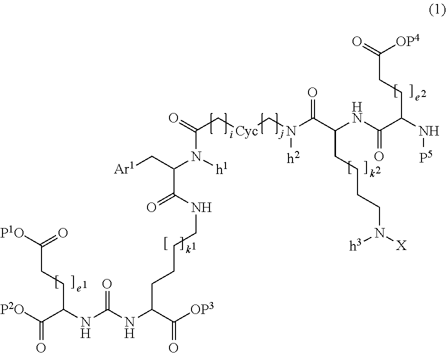
wherein
P¹, P², P³, P⁴, and P⁵ are each independently H or a protecting group;
e¹ and e² are each independently 1 or 2;
k¹ and k² are each independently 0, 1 or 2;
i is 0 or 1;
j is 0 or 1;
h¹, h², and h³ are each independently H or CH₃;
Ar¹ is an aromatic or heteroaromatic C5-12 hydrocarbon;
Cyc is an aromatic, heteroaromatic, cyclic, or heterocyclic C5-10 hydrocarbon;
X is H, a protecting group, a chelator, a detectable label, a pharmaceutically active agent, or a linker, wherein the linker is optionally attached to a chelator, a detectable label, a pharmaceutically active agent, or two or more of a chelator, a detectable label, and a pharmaceutically active agent.

2. The compound according to claim 1, wherein
P¹, P², P³, and P⁴, are each independently H or a protecting group that is a C1-7 hydrocarbon; or
P⁵ is a C2-8 acyl group, preferably comprising a C5-6 aromatic or heteroaromatic ring; or
e¹ is 1; or e² is 1; or
k¹ is 1; or k² is 1; or
i is 0; or j is 1; or
h¹ is H; or h² is H; or h³ is H; or
Ar¹ is naphthyl, phenyl, biphenyl, indolyl, benzothiazolyl, or quinoyl; or
Cyc is a C5-10 aryl, a C6-10 alkylaryl, cyclopentyl, cyclohexyl, cycloheptyl, or piperidyl.

3. The compound according to claim 1, wherein
P¹, P², P³, and P⁴, are each H or tert-butyl; or
P⁵ is benzoyl, picolinyl, nicotinyl, or isonicotinyl; or
e¹ and e² are 1; or
k¹ and k² are 1; or
i is 0 and j is 1; or
h¹, h² and h³ are H; or
Ar¹ is naphthyl; or
Cyc is phenyl, cyclopentyl, cyclohexyl, cycloheptyl, or piperidyl.

4. The compound according to claim 1, wherein
it is of general formula (1-L):

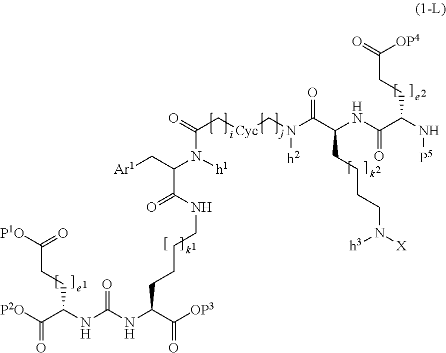

5. The compound according to claim 1, wherein X is a chelator, a detectable label, or a linker, wherein the linker is optionally attached to a chelator, a detectable label, or both a chelator and a detectable label.

6. The compound according to claim 5, wherein
the chelator is 1,4,7,10-tetraazacyclododecane-tetraacetic acid (DOTA) 1,4,7-triazacyclononane-triacetic acid (NOTA), triazacyclononane-phosphinate (TRAP), 1,4,8,11-tetraazacyclotetradecane-1,4,8,11-tetraacetic acid (TETA), N,N′-bis[2-hydroxy-5-(carboxyethyl)benzyl]ethylenediamine-N,N′-diacetic acid (HBED-CC), or diethylenetriaminepentaacetic anhydride (DTPA); and/or wherein
the detectable label is a fluorophore, a chromophore, a radiolabel, a specific isotope, a diagnostic marker, or a hapten, wherein a fluorophore is preferably fluorescein or its derivatives such as FITC or Tokyo green, ASP (preferably 4-(4-(didecylamino) styryl)-N-methylpyridinium iodide), rhodamine, Cyanine5 (also known as Cy5), Cyanine5.5 (also known as Cy5.5), Cyanine7 (also known as Cy7), sulfoCyanine7 (also known as sulfoCy7), Cyanine7.5 (also known as Cy7.5), IRDye 700DX, IRDye 800CW, IRDye 800ZW, Alexa660, Alexa680, Alexa700, Alexa750, Alexa790, DyLight 755, DyLight 800 Fluoprobes 752, FluoProbes 782, calcein, any other Alexa-label, any other Cyanine-label, and sulfonated or otherwise modified variants of any of these fluorophores.

7. The compound according to claim 5, wherein the linker comprises an amino acid, an oligo (ethylene glycol), or a C2-12 hydrocarbon, wherein the linker comprises at least one functional group for further modification.

8. The compound according to claim 5, wherein the compound comprises both a chelator and a detectable label.

9. The compound according to claim 1, wherein the compound is selected from compounds of general formula (1) wherein:
P¹, P², P³, and P⁴, are H; P⁵ is nicotinyl; e¹ and e² are 1; k¹ and k² are 1; i is 0; j is 1; h¹, h², and h³ are H; Ar¹ is naphthyl; Cyc is phenyl; X is H;
P¹, P², P³, and P⁴, are H; P⁵ is nicotinyl; e¹ and e² are 1; k¹ and k² are 1; i is 0; j is 1; h¹, h², and h³ are H; Ar¹ is naphthyl; Cyc is phenyl; X is lysine (—C(═O)C(NH₂)(CH₂)₄NH₂);
P¹, P², P³, and P⁴, are H; P⁵ is nicotinyl; e¹ and e² are 1; k¹ and k² are 1; i is 0; j is 1; h¹, h², and h³ are H; Ar¹ is naphthyl; Cyc is phenyl; X is-DOTA-lysine (—C(═O)C(NH [DOTA])(CH₂)₄NH₂);
P¹, P², P³, and P⁴, are H; P⁵ is nicotinyl; e¹ and e² are 1; k¹ and k² are 1; i is 0; j is 1; h¹, h², and h³ are H; Ar¹ is naphthyl; Cyc is phenyl; X is-IRDye700DX-lysine (—C(═O)C(NH₂)(CH₂)₄NH [IRDye700DX]);
P¹, P², P³, and P⁴, are H; P⁵ is nicotinyl; e¹ and e² are 1; k¹ and k² are 1; i is 0; j is 1; h¹, h², and h³ are H; Ar¹ is naphthyl; Cyc is phenyl; X is-DOTA-D-IRDye700DX-lysine (—C(═O)C(NH [DOTA])(CH₂)₄NH [IRDye700DX]);
P¹, P², P³, and P⁴, are H; P⁵ is benzoyl; e¹ and e² are 1; k¹ and k² are 1; i is 0; j is 1; h¹, h², and h³ are H; Ar¹ is naphthyl; Cyc is phenyl; X is H;
P¹, P², P³, and P⁴, are H; P⁵ is benzoyl; e¹ and e² are 1; k¹ and k² are 1; i is 0; j is 1; h¹, h², and h³ are H; Ar¹ is naphthyl; Cyc is phenyl; X is lysine (—C(═O)C(NH₂)(CH₂)₄NH₂);
P¹, P², P³, and P⁴, are H; P⁵ is benzoyl; e¹ and e² are 1; k¹ and k² are 1; i is 0; j is 1; h¹, h², and h³ are H; Ar¹ is naphthyl; Cyc is phenyl; X is □-DOTA-lysine (—C(═O)C(NH [DOTA])(CH₂)₄NH₂);
P¹, P², P³, and P⁴, are H; P⁵ is benzoyl; e¹ and e² are 1; k¹ and k² are 1; i is 0; j is 1; h¹, h², and h³ are H; Ar¹ is naphthyl; Cyc is phenyl; X is □-IRDye700DX-lysine (—C(═O)C(NH₂)(CH₂)₄NH [IRDye700DX]);
P¹, P², P³, and P⁴, are H; P⁵ is benzoyl; e¹ and e² are 1; k¹ and k² are 1; i is 0; j is 1; h¹, h², and h³ are H; Ar¹ is naphthyl; Cyc is phenyl; X is □-DOTA-□-IRDye700DX-lysine (—C(═O)C(NH [DOTA])(CH₂)₄NH [IRDye700DX]);
P¹, P², P³, and P⁴, are H; P⁵ is nicotinyl; e¹ and e² are 1; k¹ and k² are 1; i is 0; j is 1; h¹, h², and h³ are H; Ar¹ is naphthyl; Cyc is cyclohexyl; X is H;
P¹, P², P³, and P⁴, are H; P⁵ is nicotinyl; e¹ and e² are 1; k¹ and k² are 1; i is 0; j is 1; h¹, h², and h³ are H; Ar¹ is naphthyl; Cyc is cyclohexyl; X is lysine (—C(═O)C(NH₂)(CH₂)₄NH₂);
P¹, P², P³, and P⁴, are H; P⁵ is nicotinyl; e¹ and e² are 1; k¹ and k² are 1; i is 0; j is 1; h¹, h², and h³ are H; Ar¹ is naphthyl; Cyc is cyclohexyl; is □-DOTA-lysine (—C(═O)C(NH [DOTA])(CH₂)₄NH₂);
P¹, P², P³, and P⁴, are H; P⁵ is nicotinyl; e¹ and e² are 1; k¹ and k² are 1; i is 0; j is 1; h¹, h², and h³ are H; Ar¹ is naphthyl; Cyc is cyclohexyl; is □-IRDye700DX-lysine (—C(═O)C(NH₂)(CH₂)₄NH [IRDye700DX]);
P¹, P², P³, and P⁴, are H; P⁵ is nicotinyl; e¹ and e² are 1; k¹ and k² are 1; i is 0; j is 1; h¹, h², and h³ are H; Ar¹ is naphthyl; Cyc is cyclohexyl; X is □-DOTA-□-IRDye700DX-lysine (—C(═O)C(NH [DOTA])(CH₂)₄NH [IRDye700DX]);
P¹, P², P³, and P⁴, are H; P⁵ is benzoyl; e¹ and e² are 1; k¹ and k² are 1; i is 0; j is 1; h¹, h², and h³ are H; Ar¹ is naphthyl; Cyc is cyclohexyl; X is H;
P¹, P², P³, and P⁴, are H; P⁵ is benzoyl; e¹ and e² are 1; k¹ and k² are 1; i is 0; j is 1; h¹, h², and h³ are H; Ar¹ is naphthyl; Cyc is cyclohexyl; X is lysine (—C(═O)C(NH₂)(CH₂)₄NH₂);
P¹, P², P³, and P⁴, are H; P⁵ is benzoyl; e¹ and e² are 1; k¹ and k² are 1; i is 0; j is 1; h¹, h², and h³ are H; Ar¹ is naphthyl; Cyc is cyclohexyl; X is □-DOTA-lysine (—C(═O)C(NH [DOTA])(CH₂)₄NH₂);
P¹, P², P³, and P⁴, are H; P⁵ is benzoyl; e¹ and e² are 1; k¹ and k² are 1; i is 0; j is 1; h¹, h², and h³ are H; Ar¹ is naphthyl; Cyc is cyclohexyl; is □-IRDye700DX-lysine (—C(═O)C(NH₂)(CH₂)₄NH [IRDye700DX]); and
P¹, P², P³, and P⁴, are H; P⁵ is benzoyl; e¹ and e² are 1; k¹ and k² are 1; i is 0; j is 1; h¹, h², and h³ are H; Ar¹ is naphthyl; Cyc is cyclohexyl; X is □-DOTA-□-IRDye700DX-lysine (—C(═O)C(NH [DOTA])(CH₂)₄NH [IRDye700DX]);.

10.-14. (canceled)

15. Method of imaging, diagnosing, or treating cancer in a subject in need thereof, the method comprising the step of administering a compound according claim 1 to the subject.

16. The method according to claim 15, wherein the method is for the treatment of a cancer and/or a metastasis thereof wherein the cancer is a prostate cancer or a salivary gland cancer, more preferably a prostate cancer.
